(19) United States
(12) Patent Application Publication
(10) Pub. No.: US 2016/0054299 A9
(43) Pub. Date: Feb. 25, 2016

(54) PERFORIN 2 DEFENSE AGAINST INVASIVE AND MULTIDRUG RESISTANT PATHOGENS

(71) Applicant: University of Miami, Miami, FL (US)

(72) Inventors: Lesley De Armas, N. Bay Village, FL (US); Kirill Lyapichev, Miami, FL (US); Ryan McCormack, Miami, FL (US); Eckhard Podack, Coconut Grove, FL (US)

(21) Appl. No.: 14/396,473

(22) PCT Filed: Mar. 15, 2013
(86) PCT No.: PCT/US2013/032503

§ 371 Date: Oct. 23, 2014

Related U.S. Application Data
(60) Provisional application No. 61/637,455, filed on Apr. 24, 2012.

(51) Int. Cl. G01N 33/50; A61K 33/00; A61K 38/02; A61K 31/7088

(52) U.S. Cl. CPC G01N 33/502; A61K 31/7088; A61K 33/00; A61K 38/02; G01N 2333/435; G01N 2500/10

(57) ABSTRACT

Perforin-2 (P2) is expressed by fibroblasts, microglia and macrophages and was found to be responsible for killing bacteria, for example, Mycobacteria smegmatis, M. avium, Salmonellae, MRSA (drug resistant Stapholococci), E coli. Compounds identified by screening assays are selected based on the effects of these compounds on P2. Use of these compounds in the treatment of infectious diseases, in particular, bacteria and antibiotic-resistant bacteria is also provided.

### STATEMENT OF FEDERAL FUNDING

This invention was made with government support under grant number CA109094, awarded by the National Institutes of Health. The government has certain rights in the invention.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 430333seqlist.txt, a creation date of Mar. 12, 2013 and a size of 2 KB. The sequence listing filed via EFS-Web is part of the specification and is hereby incorporated in its entirety by reference herein.

### FIELD OF THE INVENTION

This invention relates to the fields of antibiotics, and drug discovery. More specifically, it relates to methods and compounds that are useful in potentiating the body's natural defenses to microbial infection.

### BACKGROUND

Perforin is a cytolytic protein found in the granules of CD8 T-cells and NK cells. Upon degranulation, perforin inserts itself into the target cell's plasma membrane, forming a pore. The cloning of Perforin by the inventors' laboratory (Lichtenheld, M. G., et al., 1988. Nature 335:448-451; Lowrey, D. M., et al., 1989. Proc Natl Acad Sci USA 86:247-251) and by Shinkai et al (Nature (1988) 334:525-527) established the postulated homology of complement component C9 and of perforin (DiScipio, R. G., et al., 1984. Proc Natl Acad Sci USA 81:7298-7302).

Both Perforin-1 and Perforin-2 (P2) are pore formers that are synthesized as hydrophilic, water soluble precursors. Both can insert into and polymerize within the lipid bilayer to form large water filled pores spanning the membrane. The water filled pore is made by a cylindrical protein-polymer.

The inside of the cylinder must have a hydrophilic surface because it forms the water filled pore while the outside of the cylinder needs to be hydrophobic because it is anchored within the lipid core. This pore structure is thought to be formed by an amphipathic helix (helix turn helix). It is this part of the protein domain, the so called MAC-Pf (membrane attack complex/Perforin) domain, that is most conserved between Perforin and C9 and the other complement proteins forming the membrane attack complex (MAC) of complement.

An mRNA expressed in human and murine macrophages (termed Mpg 1 or Mpeg 1-macrophage expressed gene) predicting a protein with a MAC/Pf domain was first described by Spilsbury (Blood (1995) 85:1620-1629). Subsequently, the same mRNA (named MPS-1) was found to be upregulated in experimental prion disease. The group of Desjardin analyzed the protein composition of phagosome membranes isolated from macrophages fed with latex beads by 2D-gel electrophoresis and mass spectrometry (J Cell Biol 152:165-180, 2001). The authors found protein spots corresponding to the MPS-1 protein. Mah et al analyzed abalone mollusks and found an mRNA in the blood homologous to the Mpeg1 gene family (Biochem Biophys Res Commun 316:468-475, 2004) and suggested that predicted protein has similar functions as CTL perforin but that it is part of the innate immune system of mollusks.

### SUMMARY

Described herein are compositions and methods relating modulation of P2 expression or activity, including those useful for treating microbial infections.

### BRIEF DESCRIPTION OF THE DRAWINGS

FIGS. 1A-1C show that Perforin-2 enhances the bactericidal effects of ROS and NO. Intracellular killing was inhibited by blocking ROS with 10 mM NAC, NO with 10 mM L-NAME, or P2 with a pool of 3 P2-specific siRNAs. The gentamycin protection assay was carried out as in FIGS. 2A-2F. 24 h after transfection with P-2 siRNA or scrambled siRNA. PEM were infected with (FIG. 1A) E. coli, (FIG. 1B) S. typhimurium, or (FIG. 1C) M. smegmatis. Inhibitors were added 1 h before infection and maintained throughout assay. Cells were lysed and CFU determined at 1 h and 5 h post-infection. Data shown are representative of 3 experiments with 2 replicates; asterisks denote significance (p<0.05) based on students t-test.

FIGS. 2A-2F show that Perforin-2 is a pore-forming protein. (FIG. 2A) Domain structure of P-2; TM, transmembrane domain; Cyto, cytoplasmic domain. Electron micrograph of polymerized perforin-2 membrane lesions negatively stained with neutral Na phosphotungstic acid. (FIG. 2B) Overview of membrane-associated, polymerized P-2. Note the stain-filled pores with internal diameters of 9.2 nm. (FIG. 2C) Incompletely polymerized complexes (arrows) at higher magnification. (FIG. 2D) The arrow points to a rare oblique view of a poly-P2 complex in an oblique view delineating the three-dimensional shape. (FIGS. 2E, 2F) Two types of side view of membrane-associated poly-P2. (FIG. 2G) Membrane-associated Perforin-1 (P-1) from CTL 3 stained with uranyl formate for comparison at the same magnification as Perforin-2 in FIG. 2A; note the larger diameter of the pore in P-1 (16 nm) compared to P-2 (9.2 nm).

FIGS. 3A-3I show that Perforin-2 kills intracellular Methicillin-resistant S. aureus (MRSA), S. typhimurium, M. avium, M. smegmatis, and E. coli in macrophages and microglia. Cells were infected as indicated in methods, washed to remove remaining extracellular bacteria, and gentamycin was added. Intracellular survival was measured by lysis of cells at the time indicated and determination of colony forming units (CFU) (FIGS. 3A-3D): P-2 siRNA pool knockdown of P-2 in RAW, PEM and BV-2 microglia cells allows intracellular survival of pathogenic bacteria that are otherwise killed. (FIG. 3E): Knockdown of P-2, Western blot analysis of P-2 protein expression in RAW cells following P-2 siRNA treatment. (FIGS. 3F and 3G): Overexpression of P-2-RFP fusion protein leads to enhanced killing of bacteria compared to vector control. (FIG. 3H): Knockdown of endogenous P-2 and complementation with P-2-RFP fusion protein in macrophages restores killing activity against M. smegmatis. (FIG. 3I): Western blot analysis of P-2 and P-2-RFP expression in P-2-complementation assay in RAW cells. Graphs shown are representative of 3 or more experiments with 3 replicates. Asterisks represent significant (p<0.05) differences as determined using student's t-test.

FIGS. 4A-4D show the intracellular Perforin-2-GFP localization in macrophages. RAW cells were transfected with P-2-GFP and 24 hours later fixed and stained for various cellular organelles. P-2-GFP colocalizes with ER (FIG. 4A) and Golgi membranes and trans-Golgi network (FIG. 4B), but not with plasma membrane (FIG. 4C) or lysosomes (FIG. 4D). The lower panels in the images show the indicated section in overlay (left panel) and as single color images. Colocalization appears yellow in the overlay.

FIGS. 5A, 5B show the quantitation of P-2 knock down at RNA and protein levels. siRNA mediated knockdown of P2 in FIG. 5A, RAW cells and FIG. 5B, PEM compared to scramble control. Bar graph shows P-2 relative mRNA expression in each cell type determined by quantitative TAQMAN™ RT-PCR. P-2 mRNA levels were normalized to GAPDH. Western blot analysis shows that protein levels correspond to mRNA expression in treated samples. Cells were harvested for analyses 24 hr post-transfection.

FIG. 6 shows that inhibitors of ROS and NO do not directly inhibit bacterial growth. E. coli, S. typhimurium and M. smegmatis were grown in IMDM+10% FBS in the presence or absence of NAC (10 mM) or L-NAME (10 mM) for 5 hours. Bacterial growth was measured by spectrophotometer at an OD of 600 nM at 1 and 4 hours after addition of inhibitors to the culture medium.

FIG. 7 shows the phylogenetic conservation of Perforin-2. Alignment of predicted protein sequences from several species (Vector NTI, Invitrogen). The MACPF and P-2 domains are boxed, the transmembrane domain boxed and highlighted in yellow. The conserved tyrosine and serine in the cytoplasmic domain are highlighted in pink and grey, respectively. Red font and yellow highlight indicates identity in all species, blue highlight indicates identity in four or more sequences, green indicates conservative replacement.

FIG. 8 shows the validation of P-2-GFP transfection and protein expression. Western blot analysis of transfected P-2-GFP expression in 293 cells; P-2-GFP was detected using polyclonal antiserum raised against the cytoplasmic domain of P-2 (P-2 cyto), a commercial peptide antiserum (P-2 Abcam), and an anti-GFP antibody. P-2-GFP migrated at the expected size of approximately 110 kD.

FIGS. 9A-9D show that P-2 mediates intracellular bactericidal activity in macrophage, dendritic and microglia cells and cell lines. BMDM/DC were differentiated from murine bone marrow for 10 days in the presence GM-CSF and then stimulated with (FIG. 9A) LPS (1 mg/ml) and IFNγ (100 U/ml) or (FIG. 9B) poly(I:C) (3 mg/ml) for 48 hr. (FIG. 9C) BV2 microglial cells were stimulated for 24 hr. with IFNγ (100 U/ml). (FIG. 9D) RAW cells were stimulated for 24 hr with LPS (1 ng/ml) and IFNγ (100 U/ml). Cells were harvested at the indicated time points and analyzed for P-2 message expression by TAQMAN™ RT-PCR, relative to GAPDH.

FIGS. 10A-10E show that knockdown of P2 inhibits intracellular bactericidal activity. (FIGS. 10A-10C) RAW cells treated with P-2 siRNA (dashed bars) or scramble siRNA control (solid black bars) were infected with M. smegmatis, MRSA, and S. typhimurium, respectively at MOI 10. (FIGS. 10D and 10E) PEM and BMDM/DC were treated with P2 siRNA or scramble siRNA control and infected with M. smegmatis at MOI 10. Surviving intracellular bacteria were determined in the gentamycin protection assay and enumerated by CFU assay at the indicated time points. Graphs shown are representative of at least 3 experiments per cell:bacteria combination. Error bars represent s.e.m. of 2-3 replicates. Asterisks represent significant differences using Student's t-test.

FIGS. 11A-11F show the overexpression of P-2-RFP increases intracellular killing activity. The indicated cells were transfected with a vector containing the P-2-RFP fusion protein (black bars) or control empty vector (hatched bars) and infected with the indicated bacteria at MOI 10 as described in the Materials and Methods. Surviving bacteria were enumerated by CFU assay at the indicated time points. Graphs shown are representative of at least 3 experiments per cell:bacteria combination. Error bars represent s.e.m. of 2-3 technical replicates. Asterisks represent significant differences using Student's t-test.

FIGS. 12A, 12B show that P-2-RFP transfection into cells with endogenous P-2 knock down restores intracellular bactericidal activity. FIG. 12A, BMDM/DC or FIG. 12B, RAW cells were co-transfected with a siRNA targeting the 3′UTR of P-2 and a vector containing the P-2-RFP fusion protein or empty vector alone and infected with the indicated bacteria as described in the Materials and Methods. Red lines indicate the effect of P2RFP on intracellular bacterial survival compared to the RFP vector control and black lines represent the effect of P2 siRNA treatment (3′UTR targeting only) on bacterial survival compared to scrambled siRNA control within the same experiment. Error bars represent s.e.m. of 3-4 individual experiments with 2-3 replicates.

FIGS. 13A-13C show that P-2 does not co-localize with early endosome markers or the nucleus. (FIG. 13A) Differential staining patterns of P2-GFP (left panel) and GFP (right panel) transiently transfected and expressed in RAW cells. P-2-GFP does not co-localize with (FIG. 13B) early endosomes (EEA-1, red) or (FIG. 13C) the nucleus (Hoechst, red). Images were taken using a Leica SP5 inverted confocal microscope and 40× objective.

FIGS. 14A-14J show that Perforin-2 mRNA is upregulated by type 1- and type 2-interferon. Murine embryonic fibroblasts (FIG. 14A), a rectal cancer cell line (FIG. 14B), myoblast cell line (FIG. 14C), and an ovarian cell line (FIG. 14D) were treated for 14 hours with 100 U/ml interferon-α, interferon-β, and interferon-γ treated. LPS was treated at 1 ng/ml; IL-1α at 10 U/ml, IL-1β at 1 ng/ml, and TNFα at 20 ng/mL. A human embryonic kidney cell line (FIG. 14E) and cervical cancer cell line (FIG. 14F) were treated with human IFN-α at 150 U/ml, IFN-β and IFN-γ at 100 U/ml. The BV2 microglial cell line was stimulated with 100 U/ml of murine Interferon-γ for 14 hours to upregulate P-2 mRNA and protein. mEF treated with IFN-γ for 14 hours (mEF), or IFN-γ for 14 hours followed by 1 hour of treatment with 25 mM MG-132 (mEF+MG132) (FIG. 14G). Human primary keratinocytes were analyzed for protein expression with indicated recombinant human IFN treatment (FIG. 14H). mEF were treated with E. coli or M. smegmatis. At indicated time points, cells were harvested and analyzed for P-2 mRNA and compared to uninfected controls by TAQMAN™ PCR. (FIG. 14I) mEF were either not stimulated, or stimulated for 14 hours with IFN-γ at 100 U/ml. After stimulation, mEF were infected with M. smegmatis and analyzed for colony forming units at indicated time points.

FIGS. 15A-15F show the poly-perforin-2 pores on bacteria. mEF were treated with murine Interferon-γ for 14 hours, and then infected which methicillin-resistant S. aureus (FIGS. 15A, 15B) or M. smegmatis (FIGS. 15D, 15E). After 5 hours of infection, mEFs were lysed with detergent, and intact bacteria were harvested and negatively stained for transmission electron microscopy. HEK293 cell membranes overexpressing P-2 cDNA were processed to serve as a positive control (FIG. 15C). Membrane Attack Complex (MAC) of complement pores on E. coli are shown for comparison19 (FIG. 15F).

FIGS. 16A-16L show that knock down of endogenous Perforin-2 enhances intracellular bacterial growth. Mouse rectal carcinoma CMT-93 was transiently transfected with a P-2 siRNA pool, or with P-2-RFP. Scramble siRNA or RFP were also transfected and analyzed to serve as controls for P-2 siRNA and P-2-RFP respectively. All transfections were performed 24 hours prior to infection. 14 hours prior to infection, cells were incubated with 100 U/ml of species specific IFN-γ. At indicated time points after infection, cells were lysed and plated for CFU analysis. (FIGS. 16A-16D) mEF were transfected with P-2siRNA and complemented with siRNA resistant P-2-RFP cDNA 24 hours prior to infection. 14 hours prior to infection, interferon-γ was added. mEF were infected with late log phase S. typhimurium. At indicated time points mEFs were lysed and analyzed for colony forming units. Mouse astrocytes (FIG. 16F), human pancreatic cancer (FIG. 16G), Human bladder cancer (FIG. 16H), Mouse myoblast (FIG. 16I), human cervical cancer (FIG. 16J), Mouse ovarian cancer (FIG. 16K), Human umbilical cord endothelial vein (FIG. 16L) were transfected with species specific P-2 siRNA or scramble siRNA 24 hours prior to infection, and stimulated with species specific IFN-γ 14 hours prior to infection. Cells were infected with indicated bacterium and lysed at indicated time points for CFU determination.

FIGS. 17A-17J show that P-2 increases susceptibility of intracellular bacteria to lysozyme. (FIGS. 17A-17C) Representative images taken by phase light microscopy (50× magnification) of plated M. smegmatis micro-colonies on Middlebrook 7H11 agar plates. (FIG. 17A) M. smegmatis plated prior to incubation with mEF (FIG. 17B) M. smegmatis plated after 5-hour infection with IFN-γ preactivated mEF with 30 min control (no lysozyme) incubation on ice. (FIG. 17C) M. smegmatis plated after 5-hour infection with IFN-γ preactivated mEF with 30-minute lysozyme incubation on ice after mEF lysis. (FIG. 17D) Quantification of lysozyme effect on mEFs infected with M. smegmatis, percentages are derived from each plated sample at 5 hours infection. Results consist of 5 experiments with technical replicates in each experiment. 1000 bacteria were counted and differentiated between plump and normal morphology and the percentage of plump is reported. (FIGS. 17E-17G): mEFs were transfected 24 hours prior to infection with scramble siRNA (FIG. 17E), P-2 siRNA (FIG. 17F), and P-2-RFP (FIG. 17G) and stimulated with IFN-γ for 14 hours, then infected with MRSA. At the indicated time points, eukaryotic cells were lysed to harvest intracellular bacterium and divided into six equal fractions. From these equal fractions of bacterial lysates, half were treated with lysozyme with the remainder given buffer. After a 30-minute treatment on ice to allow for lysozyme activity, bacteria were plated to analyze the effect of lysozyme. (FIGS. 17H-17J) Mouse rectal carcinoma cell line, CMT-93 was transiently transfected with scramble siRNA (FIG. 17H), P-2 siRNA (FIG. 17I), or P-2-RFP (FIG. 17J) and stimulated with IFN-γ. After treatment, these cells were infected with M. smegmatis. At indicated time points, the eukaryotic cells were lysed and analyzed for lysozyme-mediated killing.

FIGS. 18A-18I show that P-2 is inducible with type 1- and type 2-interferon in human and mouse tissues. (FIGS. 18A-18D) Murine P-2 mRNA transcript is inducible after type 1 and type 2 interferon treatment in colon cell carcinoma (FIG. 18A), melanoma (FIG. 18B), primary meningeal fibroblast (FIG. 18C), primary astrocytes (FIG. 18D). (FIGS. 18E-18I) Induction of human P-2 mRNA after type 1- and type 2-interferon treatment in bladder cancer (FIGS. 18E, 18F), pancreatic cancer (FIG. 18G), primary keratinocytes (FIG. 18H), and umbilical vein endothelial cells (FIG. 18I).

FIGS. 19A-19G: show the efficiency of mRNA knockdown with P-2 siRNA. P-2 transcript was measured for the following conditions: no transfection, P-2 siRNA transfection, or scramble siRNA. All cells were stimulated for 14 hours with 100 U/ml of IFN-γ. (FIGS. 19A-19D) The following mouse lines are a representative sampling of P-2 knockdown following siRNA treatment. These include: mEF (FIG. 19A), rectal carcinoma (FIG. 19B), meningeal fibroblast (FIG. 19C), and astrocytes (FIG. 19D). In addition, the following human cell lines will also serve as a representative sampling of P-2 transcript knockdown using human P-2 specific siRNA. These include HUVEC (E), pancreatic cancer (FIG. 19F), and bladder cancer (FIG. 19G). ¥ Indicates that P-2 transcript was not detected by qRT-PCR through 45 cycles.

FIGS. 20A, 20B show that P-2 knock down allows unimpeded intracellular bacterial replication that kills eukaryotic cells. Absolute live cell counts (FIG. 20A) and viability (FIG. 20B) of siRNA-treated mEFs post-infection with M. smegmatis as determined by trypan blue exclusion. Data shown from 4 individual experiments.

FIGS. 21A-21AF show that Perforin-2 knock down enhances intracellular bacterial growth in a variety of cell types. The following cells were transiently transfected with P-2 siRNA or scramble siRNA 24 hours prior to infection: (FIGS. 21A-21C) Murine C2C12 myoblasts, (FIGS. 21D-21F) Murine ovarian MOVAC 5009, (FIGS. 21G-21I) Human HeLa cervical carcinoma, (FIGS. 21J-21L) Human Umbilical Vein Endothelial cells (HUVEC) (FIGS. 21M-21P) Murine ovarian MOVAC 5447, (FIGS. 21Q-21T) CT-26 colon carcinoma, (FIGS. 21U-21X) Murine B16F10 melanoma (FIGS. 21Y-21AB) Human MIA-PaCa-2 pancreatic cancer, (FIGS. 21AC-21AF) Human UM-UC-9 bladder cancer. 14 hours prior to infection, cells were incubated with species-specific 100 U/ml of IFN-γ. At the indicated time points after infection, cells were lysed and plated for CFU analysis.

FIGS. 22A-22C show that P-2 complementation restores killing activity in cells with knock down of endogenous P-2. mEF cells transfected with P-2 siRNA and co-transfected with siRNA resistant P-2 cDNA are able to restore killing activity against (FIG. 22A) M. smegmatis, (FIG. 22B) E. coli, and (FIG. 22C) MRSA.

FIGS. 23A-23C show that lysozyme alone does not decrease CFU. MRSA (FIG. 23A), E. coli (FIG. 23B), and M. smegmatis (FIG. 23C) were treated to determine susceptibility of these respective bacteria to the effects of lysozyme. Three separate experiments are shown with CFU counts prior to and after incubation on ice for 30 minutes, with and without lysozyme addition.

FIGS. 24A-24L show that lysozyme enhances bactericidal action of Perforin-2. Perforin-2 is able to modulate the bactericidal activity of lysozyme on previously unresponsive bacteria. Increased activity of lysozyme is presented for mEF infected with E. coli (FIGS. 24A-24C), mEF infected with M. smegmatis (FIGS. 24D-24F), CMT-93 infected with E. coli (FIGS. 24G-241), and CMT-93 infected with MRSA.

FIGS. 25A-C show that P-2 deficiency leads to uncontrolled and lethal bacterial growth. (a) Daily weight measurements from P-2+/+ (□), P2−/− (□) and P-2+/− (□) littermates. Mice received 20 mg streptomycin and 24 h later 105 or 102 S. typhimurium RL144 by oro-gastric gavage. n=10 and 15 (analysis: multiple unpaired t-tests for each row). (b) Dissemination of S. typhimurium from intestines to blood liver and spleen (analysis: Kruskal-Wallis tests). (c) Lack of control of S. typhimurium replication in genetic P2−/− PEM (□) compared to siRNA P-2 knock down PEM (□); CFU assay as described in material and methods (analysis: unpaired t=tests).

FIGS. 26A-F show that P-2 is expressed ubiquitously and is bactericidal against Salmonella typhimurium, Mycobacterium smegmatis and avium, and MRSA. (a). Western blot analysis of PMN and macrophages from human blood and western blot and CFU assay of siRNA treated HL60-derived PMN. P-2 specific (□) and scramble control (□) siRNA transfected HL60/PMN cells were infected with the indicated bacteria and CFU analyzed at the indicated times as described in methods. (b) CFU assay of IFN-□ induced intestinal epithelial CMT93 cells following infection with the bacteria as indicated. Cells were transfected with siRNA: P-2 specific (□) or scramble control (□) and with plasmid-cDNA for: P-2-RFP (□) or RFP (□) and stimulated overnight with IFN-γ prior to infection. (c) (d) P-2 induced by IFN-γ in primary human umbilical cord endothelial cells (HUVEC) or cervical epithelial cells (HeLa) kills M. smegmatis (and other bacteria—not shown). (e) Complete clearance of M. avium from RAW macrophages by P-2-RFP transfection (f) Complementation of endogenous P-2 knock down in MEF with P-2-RFP. CFU assay and western blot analysis of P-2 siRNA and P-2-RFP transfected and IFN-γ activated MEF followed by infection with Salmonella; P-2 siRNA+RFP (□), P-2 siRNA+P-2-RFP (□), and scramble siRNA+RFP (▴). Statistics by t-tests.

FIGS. 27A-E show that bacteria have mechanisms to block P-2. (a) Salmonella blocks P-2 mRNA induction in naïve MEF (not activated by IFN). Relative P-2 mRNA levels following infection with the indicated Salmonella and E. coli strains. (b) C. trachomatis blocks INF-□ mediated induction of P-2 mRNA expression in HeLa cells. Chloramphenical (Cm) treated chlamydiae induce P-2 mRNA. Data are presented as mean±standard deviation of triplicate samples. (c) Enteropathogenic E coli (EPEC) block P-2 mediated killing if they carry the CIF plasmid. (d) P-2 message levels dermis and epidermis excised from the edges of non-healing chronic ulcers. The skin samples are divided into adjacent and distant halves to chronic skin ulcers, the ratio of adjacent to distant P-2 mRNA levels is depicted. Statistics by t-tests; n=10.

FIGS. 28A-D show that P-2 translocates to the bacterium-containing vacuole. (a). Protein domain structure, predicted transmembrane orientation of P-2 within membrane vesicles, and sequence conservation of the cytoplasmic domain in mammalian species. (b) Representative confocal images of P-2 siRNA and transiently P-2-GFP (green) transfected BV2 5 min after infection with Salmonella; DNA stained by DAPI, shown in false color, white. Three vertical slices of 1.2μ representing the whole cell are shown. (c) Representative confocal images of P-2 siRNA and transiently P-2-RFP (red) transfected BV2 microglial cells infected with E. coli-GFP (green) for 5 minutes and then fixed and imaged. (d) Perinuclear localization of P-2-GFP in uninfected cells; fluorescence and corresponding phase image is shown.

FIGS. 29A-C show pore formation by Perforin-2. (a) Electron micrograph of poly-P-2 pores in Hek-293 membranes. White arrows show typical polymeric ring structures with stain-filled pores of 9.2±0.5 nm internal diameter. Black arrows point to incompletely and irregularly polymerized complexes. Negative stain with neutral Na-phosphotungstate. (c,d) Electron micrographs of polymerized P-2 membrane lesions on M. smegmatis (c) and MRSA (d) following infection of IFN-γ induced MEF. Bacterial membranes were isolated as described in methods and negatively stained for transmission electron microscopy. White arrows point to circular black, stain filled pores on the bacterial cell wall surrounded by white narrow borders putatively created by polymerizing P-2. Black arrows point to irregular polymers.

FIG. 30: (A) P-2 siRNA knock down in rectal epithelial carcinoma cells causes intracellular replication of S. typhimurium, Methicillin resistant S. aureus (MRSA, clinical isolate) and Mycobacterium smegmatis; P-2-RFP overexpression increases killing of intracellular bacteria. (B) P-2-RFP but not RFP transfection restores killing activity in mEF when endogenous P-2 is knocked-down. (C) Knock down of P-2 and complementation with P-RFP, western blots. Cells were transfected 24 hours before infection with P-2-siRNA specific for the 3′UTR of P-2 or with scrambled siRNA together with RFP or P-2-RFP, lacking the 3′UTR of endogenous P-2. At −16 h cells were incubated with 100 U/ml IFN-γ to induce P-2-RNA. At 0 h the cells were incubated for 1 h with bacteria at MoI of 30, washed to remove external bacteria and replated with gentamycin to prevent growth of extracellular bacteria. Host cells were lysed at the indicated times with NP40 and CFU determined in the lysates. Please note that knock down of P-2 by P-2 siRNA increases intracellular CFU indicating that P-2 blockade permits bacterial replication, which kills host cells (as indicated, except Salmonella). Transfection with P-2-RFP (P-2 C-terminal RFP fusion) but not RFP increases killing of intracellular bacteria (3 left panels) or restores P-2 activity when P-2 was knocked down.

FIG. 31: Intracellular bacterial replication when P-2 is knocked down and only ROS and NO are present (red circles). Good killing when P-2, ROS and NO are present (green solid spheres). Intermediate killing with P-2+NO (black solid triangles) or P-2+ROS (diamonds). Effectiveness of P-2 knock-down (right panel). Cells: IFN treated, thioglycolate elicited, peritoneal macrophages and S. typhimurium.

FIG. 32: (a) P-2-GFP, RASA2 and LC3-RFP colocalize on endocytosed bacteria. Two top panels: IFN activated BV2 stained with DAPI (DNA), RASA-2 antibody and transfected with P-2-GFP and LC3-RFP. All other panels: BV2 infected at 100:1 with Salmonella typhimurium for 5 min and then fixed with para-formaldehyde. Fluorescent label as indicated in the panels. Note one extracellular bacterium (arrow) and several killed intracellular bacteria (asterisks) as indicated by DAPI staining and P-2-GFP. Colocalization of RASA2 and LC3-RFP. (b) Trans-location of P-2-RFP to E. coli-GFP containing vacuole (arrow in phase) within 5 minutes of infection of IFN activated BV-2.

FIG. 33: Colocalization of P-2 and RASA2 in/on perinuclear membranes in resting uninfected RAW cells transfected with P-2-GFP and stained with RASA2 antibody.

FIG. 34: (a) Activation of mEF with IFN increases P-2 mRNA expression (left, Taqman PCR) and enhances intracellular killing of Mycobacteria (right, gentamycin protection assay). (b) Live WT Salmonella suppress P-2 induction in mEF. Heat killed and PhoP mutant Salmonella and E. coli K12 induce P-2. mEF were infected and after 1 h washed and plated in gentamycin.

FIG. 35: (a) siRNA knock down of RASA2 inhibits intracellular killing of Mycobacteria by BV2 and allows intracellular replication (solid bars). (b) Immunoprecipitation of P-2-GFP with anti-GFP and pull down of RASA2 in RAW cells transfected with P-2-GFP.

FIG. 36: mEF kill intracellular MRSA and generate cell wall damage similar to poly P-2 on eukaryotic membranes. Left panel MRSA cell walls obtained 4 hours after infection by detergent lysis of mEF; negative stain with uranyl formate. Right: poly P-2 complexes on HEK293 membranes; negative stained with Na phospho-tungstate Scale bars: 400 Å. Note similar diameter (90 Å) of cell wall/membrane pores.

FIG. 37: siRNA knock down of Atg14L or P-2 blocks killing and enables replication of intracellular bacteria in BV2 microglia. Intracellular killing or survival of mycobacteria determined in the gentamycin protection assay.

FIG. 38: siRNA knock down of (a) P-2, (b) Atg14L, (c) Atg16L, (d) Atg5 enables Salmonella replication in mEF. In mEF P-2 is preventing intracellular replication of Salmonella that replicate when P-2 is knocked down. Identical effects have been reported for knock down of autophagy in agreement with our data presented here.

FIG. 39: 3-MA inhibits vps34 and allows replication of Salmonella in mEF (blue line, in c) similar to P-2-knock-down (red line in a-c). Bafilomycin (blue, panel b) similar to scramble siRNA (bleu, panel a) does inhibit P-2 mediated bacteriostasis of Salmonella.

FIG. 40: Model of Perforin-2 mechanism.

### DETAILED DESCRIPTION

### Definitions

Before describing the invention in greater detail the following definitions are set forth to illustrate and define the meaning and scope of the terms used to describe the invention herein:

By the term “modulate,” it is meant that any of the mentioned activities, are, e.g., increased, enhanced, agonized (acts as an agonist), promoted, upregulated, decreased, reduced, suppressed, blocked, downregulated, or antagonized (acts as an antagonist). Modulation can “increase” or “upregulate” activity more than 1-fold, 2-fold, 3-fold, 5-fold, 10-fold, 100-fold, etc., over baseline values. Modulation can also decrease or downregulate activity below baseline values. As used herein a “decrease” or “downregulation” is meant at least a 1%, 5%, 10%, 20%, 30%, 40%, 50%, 60%, 70%, 80%, or 90% decrease relative to an appropriate control. Modulation can also normalize an activity to a baseline value.

As used herein, a “pharmaceutically acceptable” component/carrier etc., is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response.

The terms “patient” or “individual” are used interchangeably herein, and refers to a mammalian subject to be treated, with human patients being preferred. In some cases, the methods of the invention find use in experimental animals, in veterinary application (e.g., in cats, dogs, horses, cows, sheep, and pigs), and in the development of animal models for disease, including, but not limited to, rodents including mice, rats, and hamsters; and primates.

“Treatment” is an intervention performed with the intention of preventing the development or altering the pathology or symptoms of a disorder. Accordingly, “treatment” refers to both therapeutic treatment and prophylactic or preventative measures.

“Target molecule” includes any molecule which affects or modulates the expression, function or activity of Perforin-2. This includes those in Table 1, for example, and those which are yet to be identified.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3rd ed. Cold Spring Harbor Laboratory Press: Cold Spring Harbor, New York; Sambrook et al., (1989) Molecular Cloning: A Laboratory Manual. 2nd ed. Cold Spring Harbor Laboratory Press: Cold Spring Harbor, New York; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4th ed. Wiley-Liss; among others. The Current Protocols listed above are updated several times every year.

### Therapeutic Compositions

Embodiments are directed to identification of compounds which modulate the expression, function or activity of Perforin-2, both in vitro and in vivo. One of the approaches taken was to identify molecules which modulate the expression, function or activity of P2. These molecules were identified based on yeast hybrid systems, described in detail in the examples section which follows. Modulation of Perforin-2 expression, functions or any activities by candidate therapeutic agents will be effective in the prevention or treatment of infection by pathogenic organisms, especially those which have developed and those that will likely develop resistance to antibiotics, for example, bacteria.

In one embodiment, a method of modulating function, activity or expression of Perforin-2 (P2) in vitro or in vivo comprising: contacting a cell in vitro or administering to a patient, an effective amount of at least one agent which modulates function, activity or expression of one or more target molecules associated with P2 expression, function or activity is provided.

In another embodiment, a method is provided to identify at least one agent that modulates expression, function or activity of Perforin-2, the method comprising: contacting a cell expressing one or more target molecules associated with Perforin-2 expression, function or activity with the at least one agent; measuring the expression, function or activity of the one or more target molecules associated with Perforin-2 expression, function or activity; and comparing the expression, function or activity of the one or more target molecules with a control, wherein contact with the at least one agent modulates the expression, function or activity of said one or more target molecules thereby identifying an agent that modulates expression, function or activity of Perforin-2.

In some embodiments, the one or more target molecules associated with P2 function, activity or expression comprise: src, ubiquitin conjugating enzyme E2M, GAPDH, P21RAS/gap1m, Galectin 3, ubiquitin C (UCHL1), proteasomes, vps34, ATG5, ATG7, ATG9L1, ATG14L, ATG16L, LC3, Rab5, fragments, or associated molecules thereof. Molecules associated with the target molecules, can be any intracellular molecules involved in the various pathways that these target molecules participate, molecules which associate with these molecules, signaling pathways, molecules which regulate transcription and translation of these target molecules.

In other embodiments, the at least one agent upregulates the expression, function or activity of one or more target molecules associated with Perforin-2 expression, function or activity. Alternatively, the at least one agent downregulates the expression, function or activity of one or more target molecules associated with Perforin-2 expression, function or activity.

In some embodiments, the P2 expression, function or activity is upregulated by administration of the at least one agent which upregulates the function, activity or expression of the one or more target molecules associated with P2 function, expression or activity.

In some embodiments, the P2 expression, function or activity is downregulated by administration of the at least one agent which downregulates the function, activity or expression of the one or more molecules associated with P2 function, expression or activity.

In some cases it may be desired to upregulate some of the target molecules and to inhibit or keep constant the functions, activities or expressions of other target molecules. Thus, in some embodiments, the P2 expression, function or activity is upregulated by administration of at least one agent which independently upregulates or downregulates the function, activity or expression of at least two molecules associated with P2 function, expression or activity.

In other embodiments, the P2 expression, function or activity is downregulated by administration of at least one agent which independently upregulates or downregulates the function, activity or expression of at least two molecules associated with P2 function, expression or activity.

In another embodiment, the P2 expression, function or activity is upregulated by administration of a combination of at least two agents which independently upregulate or downregulate the function, activity or expression of at least two molecules associated with P2 function, expression or activity.

In some embodiments, the P2 expression, function or activity is downregulated by administration of a combination of at least two agents which independently upregulate or downregulate the function, activity or expression of at least two molecules associated with P2 function, expression or activity.

In other embodiments, modulation of P2 expression, function or activity comprises an optional step of administering an agent which directly modulates expression, function or activity of the P2 molecule. Such molecules, can be ones which inhibits transcription or translation of P2. See, for example, US Publication No.: 20090142768, incorporated herein by reference in its entirety.

In other embodiments, an agent comprises: a small molecule, protein, peptide, polypeptide, modified peptides, modified oligonucleotides, oligonucleotide, polynucleotide, synthetic molecule, natural molecule, organic or inorganic molecule, or combinations thereof.

In other aspects, the one or more target molecules associated with Perforin-2 expression, function or activity is from an infectious organism. In some embodiments, the infectious organism is a bacterium. In specific embodiments, the bacterium can be Salmonella typhimurium or Escherichia coli. In yet other embodiments, the one or more target molecules can be PhoP or deamidase.

In such cases where the one or more target molecules associated with Perforin-2 expression, function or activity are from an infectious organism, downregulation of the expression, function or activity of one or more target molecules can upregulate the expression, function or activity of Perforin-2. Alternatively, upregulation of the expression, function or activity of one or more target molecules can downregulate the expression, function or activity of Perforin-2.

Another aspect of the invention relates to methods of screening for compounds or candidate therapeutic agents which modulate the expression, function or activity of molecules which in turn modulate the expression, function or activity of Perforin 2. The compounds may, for example, induce a cell to express Perforin 2 protein, allow for assembly of the P2, allow for correct assembly, allow for the translocation of the P2, etc. Preferred candidate therapeutic agents increase P2 expression in immunological cells such as macrophages which will enhance their anti-microbial efficacy.

As such, these embodiments are directed to methods for screening compounds that are effective in increasing expression, function or activity of P2, such as for example, increasing translation of P2 mRNAs in cells. Preferably, such compounds will target molecules associated with P2. In its most basic form, such methods involve exposing cells that express certain target molecules and an endogenous or exogenous Perforin 2 gene or cDNA, respectively, with a test compound and determining whether an increase in Perforin 2 protein production results.

In one embodiment, a method of identifying a candidate therapeutic agent comprising: contacting a cell expressing one or more target molecules comprising: src, ubiquitin conjugating enzyme E2M, GAPDH, P21RAS/gap1m, Galectin 3, ubiquitin C (UCHL1), proteasomes, or fragments thereof; measuring the expression, function or activity of the molecules; comparing the expression, function or activity of the molecules with a control. Preferably, the candidate therapeutic agent modulates the expression, function or activity of one or more of the target molecules. In other embodiments, the candidate therapeutic agent modulates the expression, function or activity of a plurality of target molecules. Preferably, the candidate therapeutic agent upregulates the expression, function or activity of one or more of the target molecules. In some aspects, the candidate therapeutic agent downregulates the expression, function or activity of one or more of the target molecules.

In preferred embodiments, the modulation of the expression, function or activity of one or more target molecules modulates the expression, function or activity of Perforin-2 (P2) molecules. Preferably, the upregulation of the expression, function or activity of one or more target molecules upregulates the expression, function or activity of Perforin-2 (P2) molecules. In some aspects, the downregulation of the expression, function or activity of one or more target molecules downregulates the expression, function or activity of Perforin-2 (P2) molecules.

In another embodiment, the method of identifying a candidate agent that modulates expression, function or activity of Perforin-2 comprises: contacting an assay surface with one or more target molecules comprising src, ubiquitin conjugating enzyme E2M, GAPDH, P21RAS/gap1m, Galectin 3, ubiquitin C (UCHL1), proteasomes, vps34, ATG5, ATG7, ATG9L1, ATG14L, ATG16L, LC3, Rab5, PhoP, deamidase, fragments or associated molecules thereof; contacting the target molecules with one or more candidate agents and identifying the agents which bind or hybridize to one or more target molecules or associated molecules thereof; and assaying the one or more candidate agents for modulation of expression, function or activity of Perforin-2, thereby identifying a candidate agent.

Once a therapeutic agent is deemed to be a candidate by measuring its effects on the target molecules, the agent is further screened against Perforin-2 molecules. These could be by peptides or oligonucleotides disposed on an assay surface, for example, a biochip, or it could be in the form of cells which express P2. Preferred candidate therapeutic agent upregulates the expression, function, or activity of P2 molecules and also increase the killing of infectious organisms such as bacteria. In some aspects, an identified candidate therapeutic agent downregulates the expression, function, or activity of P2 molecules.

In other embodiments, the assays for assaying the expression, function or activity of P2 molecules comprise: cellular assays, immuno-assays, yeast hybrid system assays, hybridization assays, nucleic acid based assays, high-throughput screening assays or combinations thereof. In some aspects, the identified candidate agents are also assayed for inhibition of replication, inhibition of growth, or death of an infectious organism, e.g. bacteria.

In methods where cells are used, a control cell, a test cell comprising one or more vectors expressing a target molecule, cells whereby the target molecules are endogenous, cells having a Perforin 2 expression vector, or any combination, are provided. In this method, the test cell is contacted with a test compound, whereas the control cell is not. The technician can then identify test compounds as potential therapeutic agents if when the test cell produces more reporter protein than the control cell grown in the absence of the test compound, if the expression of the target molecule is used as the output readout parameter for identifying a potential or candidate therapeutic agent. Such test compounds are presumed to be effective antibiotic or even anti-cancer compounds that potentiate the body's own immune system in its fight against microbes and tumor cells. The test can also include a further determination at a functional level, by which cells are then tested for their ability to either kill microbes such as bacteria in co-culture.

Examples of infectious bacteria comprise without limitation: Escherichia coli, Enteropathogenic E. coli (EPEC), Methicillin-resistant Staphylococcus aureus (MRSA), Mycobacterium avium intracellulare (M. avium), Salmonella typhimurium (S. typhimurium), Helicobacter pylori, Borrelia burgdorferi, Legionella pneumophila, Mycobacterium tuberculosis, Mycobacterium bovis (BCG), Mycobacterium avium, Mycobacterium smegmatis, Mycobacterium intracellulare, Staphylococcus aureus, Neisseria gonorrhoeae, Neisseria meningitidis, Listeria monocytogenes, Streptococcus pyogenes, Streptococcus pneumoniae, Haemophilus influenzae, Moraxella catharralis, Klebsiella pneumoniae, Bacillus anthracis, Corynebacterium diphtheriae, Clostridium perfringens, Clostridium tetani, Enterobacter aerogenes, Klebsiella pneumoniae, Pasturella multocida, Escherichia coli (E. coli) and Treponema pallidum; infectious fungi like: Cryptococcus neoformans, Histoplasma capsulatum, Coccidioides immitis, Blastomyces dermatitidis, Candida albicans; and infectious protists like: Plasmodium falciparum, Trypanosoma cruzi, Leishmania donovani and Toxoplasma gondii; as well as infectious fungi such as those causing e.g., histoplasmosis, candidiasis, cryptococcosis, blastomycosis and eocidiodomycosis; as well as Candida spp. (i.e., C. albicans, C. parapsilosis, C. krusei, C. glabrata, C. tropicalis, or C. lusitaniae); Torulopus spp. (i.e., T. glabrata); Aspergillus spp. (i.e., A. fumigalus), Histoplasma spp. (i.e., H. capsulatum); Cryptococcus spp. (i.e., C. neoformans); Blastomyces spp. (i.e., B. dermatilidis); Fusarium spp.; Trichophyton spp., Pseudallescheria boydii, Coccidioides immits, and Sporothrix schenekii.

### Screening Assays

The assay for drug screening for Perforin 2 (P2) expression, function, or activity of P2 is based on the identification of molecules which increase or decrease the antibacterial effects of P2. As such, any molecule which affects any of the molecules which interact and affect the antibacterial activity of P2, would be candidate agents for therapy.

In one embodiment, screening comprises contacting each cell culture expressing the target molecules with a diverse library of member compounds. The compounds or “candidate therapeutic agents” or “agents” can be any organic, inorganic, small molecule, protein, antibody, aptamer, nucleic acid molecule, or synthetic compound.

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Chemical Libraries:

Developments in combinatorial chemistry allow the rapid and economical synthesis of hundreds to thousands of discrete compounds. These compounds are typically arrayed in moderate-sized libraries of small molecules designed for efficient screening. Combinatorial methods, can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with β-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

Small Molecules:

Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

In a preferred embodiment, the compounds are assayed against the cells comprising either vectors with inducible or noninducible promoters expressing one or more of the target molecules and/or P2 as high throughput screening. The cells used can also be cells which endogenously express the target molecules and/or P2. The reporter molecules can be the same or different molecules, however, the reporter molecules are preferably different.

In another aspect, the present invention provides a method for analyzing cells comprising providing an array of locations which contain multiple cells wherein the cells contain one or more fluorescent or luciferase reporter molecules; scanning multiple cells in each of the locations containing cells to obtain signals from the reporter molecule in the cells; converting the signals into digital data; and utilizing the digital data to determine the distribution, environment or activity of the reporter molecule within the cells.

A major component of the new drug discovery paradigm is a continually growing family of fluorescent and luminescent reagents that are used to measure the temporal and spatial distribution, content, and activity of intracellular ions, metabolites, macromolecules, and organelles. Classes of these reagents include labeling reagents that measure the distribution and amount of molecules in living and fixed cells, environmental indicators to report signal transduction events in time and space, and fluorescent protein biosensors to measure target molecular activities within living cells. A multiparameter approach that combines several reagents in a single cell is a powerful new tool for drug discovery.

This method relies on the high affinity of fluorescent or luminescent molecules for specific cellular components. The affinity for specific components is governed by physical forces such as ionic interactions, covalent bonding (which includes chimeric fusion with protein-based chromophores, fluorophores, and lumiphores), as well as hydrophobic interactions, electrical potential, and, in some cases, simple entrapment within a cellular component. The luminescent probes can be small molecules, labeled macromolecules, or genetically engineered proteins, including, but not limited to green fluorescent protein chimeras.

Those skilled in this art will recognize a wide variety of fluorescent reporter molecules that can be used in the present invention, including, but not limited to, fluorescently labeled biomolecules such as proteins, phospholipids, RNA and DNA hybridizing probes. Similarly, fluorescent reagents specifically synthesized with particular chemical properties of binding or association have been used as fluorescent reporter molecules (Barak et al., (1997), J Biol. Chem. 272:27497-27500; Southwick et al., (1990), Cytometry 11:418-430; Tsien (1989) in Methods in Cell Biology, Vol. 29 Taylor and Wang (eds.), pp. 127-156). Fluorescently labeled antibodies are particularly useful reporter molecules due to their high degree of specificity for attaching to a single molecular target in a mixture of molecules as complex as a cell or tissue.

The luminescent probes can be synthesized within the living cell or can be transported into the cell via several non-mechanical modes including diffusion, facilitated or active transport, signal-sequence-mediated transport, and endocytotic or pinocytotic uptake. Mechanical bulk loading methods, which are well known in the art, can also be used to load luminescent probes into living cells (Barber et al. (1996), Neuroscience Letters 207:17-20; Bright et al. (1996), Cytometry 24:226-233; McNeil (1989) in Methods in Cell Biology, Vol. 29, Taylor and Wang (eds.), pp. 153-173). These methods include electroporation and other mechanical methods such as scrape-loading, bead-loading, impact-loading, syringe-loading, hypertonic and hypotonic loading. Additionally, cells can be genetically engineered to express reporter molecules, such as GFP, coupled to a protein of interest as previously described (Chalfie and Prasher U.S. Pat. No. 5,491,084; Cubitt et al. (1995), Trends in Biochemical Science 20:448-455).

Once in the cell, the luminescent probes accumulate at their target domain as a result of specific and high affinity interactions with the target domain or other modes of molecular targeting such as signal-sequence-mediated transport. Fluorescently labeled reporter molecules are useful for determining the location, amount and chemical environment of the reporter. For example, whether the reporter is in a lipophilic membrane environment or in a more aqueous environment can be determined (Giuliano et al. (1995), Ann. Rev. of Biophysics and Biomolecular Structure 24:405-434; Giuliano and Taylor (1995), Methods in Neuroscience 27.1-16). The pH environment of the reporter can be determined (Bright et al. (1989), J. Cell Biology 104:1019-1033; Giuliano et al. (1987), Anal. Biochem. 167:362-371). It can be determined whether a reporter having a chelating group is bound to an ion, such as Ca⁺⁺, or not (Bright et al. (1989), In Methods in Cell Biology, Vol. 30, Taylor and Wang (eds.), pp. 157-192; Shimoura et al. (1988), J. of Biochemistry (Tokyo) 251:405-410; Tsien (1989) In Methods in Cell Biology, Vol. 30, Taylor and Wang (eds.), pp. 127-156).

Furthermore, certain cell types within an organism may contain components that can be specifically labeled that may not occur in other cell types. Therefore, reporter molecules can be designed to label not only specific components within specific cells, but also specific cells within a population of mixed cell types.

Those skilled in the art will recognize a wide variety of ways to measure fluorescence. For example, some fluorescent reporter molecules exhibit a change in excitation or emission spectra, some exhibit resonance energy transfer where one fluorescent reporter loses fluorescence, while a second gains in fluorescence, some exhibit a loss (quenching) or appearance of fluorescence, while some report rotational movements (Giuliano et al. (1995), Ann. Rev. of Biophysics and Biomol. Structure 24:405-434; Giuliano et al. (1995), Methods in Neuroscience 27:1-16).

The whole procedure can be fully automated. For example, sampling of sample materials may be accomplished with a plurality of steps, which include withdrawing a sample from a sample container and delivering at least a portion of the withdrawn sample to test cell culture (e.g., a cell culture wherein gene expression is regulated). Sampling may also include additional steps, particularly and preferably, sample preparation steps. In one approach, only one sample is withdrawn into the auto-sampler probe at a time and only one sample resides in the probe at one time. In other embodiments, multiple samples may be drawn into the auto-sampler probe separated by solvents. In still other embodiments, multiple probes may be used in parallel for auto sampling.

In the general case, sampling can be effected manually, in a semi-automatic manner or in an automatic manner. A sample can be withdrawn from a sample container manually, for example, with a pipette or with a syringe-type manual probe, and then manually delivered to a loading port or an injection port of a characterization system. In a semi-automatic protocol, some aspect of the protocol is effected automatically (e.g., delivery), but some other aspect requires manual intervention (e.g., withdrawal of samples front a process control line). Preferably, however, the sample(s) are withdrawn from a sample container and delivered to the characterization system, in a fully automated manner—for example, with an auto-sampler.

According to the present invention, one or more systems, methods or both are used to identify a plurality of sample materials. Though manual or semi-automated systems and methods are possible, preferably an automated system or method is employed. A variety of robotic or automatic systems are available for automatically or programmably providing predetermined motions for handling, contacting, dispensing, or otherwise manipulating materials in solid, fluid liquid or gas form according to a predetermined protocol. Such systems may be adapted or augmented to include a variety of hardware, software or both to assist the systems in determining mechanical properties of materials. Hardware and software for augmenting the robotic systems may include, but are not limited to, sensors, transducers, data acquisition and manipulation hardware, data acquisition and manipulation software and the like. Exemplary robotic systems are commercially available from CAVRO Scientific Instruments (e.g., Model NO. RSP9652) or BioDot (Microdrop Model 3000).

Generally, the automated system includes a suitable protocol design and execution software that can be programmed with information such as synthesis, composition, location information or other information related to a library of materials positioned with respect to a substrate. The protocol design and execution software is typically in communication with robot control software for controlling a robot or other automated apparatus or system. The protocol design and execution software is also in communication with data acquisition hardware/software for collecting data from response measuring hardware. Once the data is collected in the database, analytical software may be used to analyze the data, and more specifically, to determine properties of the candidate drugs, or the data may be analyzed manually.

### Pharmaceutical Compositions

Further provided are methods of treating a subject suffering from infection by an infectious disease organism comprising administration of a therapeutically effective amount of an agent which modulates the expression, function or activity of Perforin-2 or modulates the expression, function or activity of one or more target molecules associated with Perforin-2 expression, function or activity.

In preferred embodiments, a method of treating a patient suffering from an infectious disease organism comprises administering to the patient a therapeutically effective amount of an agent which modulates the expression, function or activity of one or more target molecules comprising: src, ubiquitin conjugating enzyme E2M (Ubc12), GAPDH, P21RAS/gap1m (RASA2), Galectin 3, ubiquitin C (UCHL1), proteasomes, vps34, ATG5, ATG7, ATG9L1, ATG14L, ATG16L, LC3, Rab5, Perforin-2, fragments or associated molecules thereof.

Active variants and fragments of src, ubiquitin conjugating enzyme E2M (Ubc12), GAPDH, P21RAS/gap1m (RASA2), Galectin 3, ubiquitin C (UCHL1), proteasomes, vps34, ATG5, ATG7, ATG9L1, ATG14L, ATG16L, LC3, Rab5, Perforin-2 or any associated molecules thereof can be used in the methods provided herein. Such active variants can comprise at least 65%, 70%, 75%, 80%, 85%, 90%, 91%, 92%, 93%, 94%, 95%, 96%, 97%, 98%, 99% or more sequence identity to any of the various target molecules provided herein, wherein the active variants retain biological activity and hence modulate Perforin-2 expression, function or activity.

In another preferred embodiment, a compound comprises a therapeutic agent identified by the methods embodied herein.

The invention also includes pharmaceutical compositions containing one or more of the therapeutic agents. In some embodiments, the compositions are suitable for internal use and include an effective amount of a pharmacologically active agent of the invention, alone or in combination, with one or more pharmaceutically acceptable carriers. The agents are especially useful in that they have very low, if any toxicity. The patient having a pathology, e.g. the patient treated by the methods of this invention can be a mammal, or more particularly, a human. In practice, the agents, are administered in amounts which will be sufficient to exert their desired biological activity.

The pharmaceutical compositions of the invention may contain, for example, more than one agent which may act independently of the other on a different target molecule. In some examples, a pharmaceutical composition of the invention, containing one or more compounds of the invention, is administered in combination with another useful composition such as an anti-inflammatory agent, an immunostimulator, a chemotherapeutic agent, an antibacterial agent, or the like. Furthermore, the compositions of the invention may be administered in combination with a cytotoxic, cytostatic, or chemotherapeutic agent such as an alkylating agent, anti-metabolite, mitotic inhibitor or cytotoxic antibiotic, as described above. In general, the currently available dosage forms of the known therapeutic agents for use in such combinations will be suitable.

Combination therapy (or “co-therapy”) includes the administration of a therapeutic composition and at least a second agent as part of a specific treatment regimen intended to provide the beneficial effect from the co-action of these therapeutic agents. The beneficial effect of the combination includes, but is not limited to, pharmacokinetic or pharmacodynamic coactions resulting from the combination of therapeutic agents. Administration of these therapeutic agents in combination typically is carried out over a defined time period (usually minutes, hours, days or weeks depending upon the combination selected).

Combination therapy may, but generally is not, intended to encompass the administration of two or more of these therapeutic agents as part of separate monotherapy regimens that incidentally and arbitrarily result in the combinations of the present invention. Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

The agents can be formulated according to known methods to prepare pharmaceutically useful compositions, whereby the compound is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™. (ICI Americas Inc., Bridgewater, N.J.), PLURONICS™. (BASF Corporation, Mount Olive, N.J.) or PEG.

The formulations to be used for in vivo administration must be sterile and pyrogen free. This is readily accomplished by filtration through sterile filtration membranes, prior to or following lyophilization and reconstitution.

The route of administration is in accord with known methods, e.g. injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

Formulations for oral administration in the present invention may be presented as: discrete units such as capsules, cachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavoring agents such as peppermint, oil of wintergreen, cherry flavoring and the like can also be used. It may be desirable to add a coloring agent to make the dosage form readily identifiable. Tablets may also be coated by methods well known in the art.

A tablet may be made by compression or molding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavored base, usually sucrose and acacia or tragacanth; pastilles comprising the active agent in an inert base such as gelatin and glycerin, or sucrose and acacia; and mouthwashes comprising the active agent in a suitable liquid carrier.

Parenteral formulations will generally be sterile.

Controlled or sustained release compositions include formulation in lipophilic depots (e.g. fatty acids, waxes, oils). Also comprehended herein are particulate compositions coated with polymers (e.g. poloxamers or poloxamines) and the compound coupled to antibodies directed against tissue-specific receptors, ligands or antigens or coupled to ligands of tissue-specific receptors. Other embodiments of the compositions presented herein incorporate particulate forms protective coatings, protease inhibitors or permeation enhancers for various routes of administration, including parenteral, pulmonary, nasal and oral.

When administered, compounds are often cleared rapidly from mucosal surfaces or the circulation and may therefore elicit relatively short-lived pharmacological activity. Consequently, frequent administrations of relatively large doses of bioactive compounds may be required to sustain therapeutic efficacy. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The sufficient amount may include but is not limited to from about 1 μg/kg to about 100 μg/kg, from about 100 μg/kg to about 1 mg/kg, from about 1 mg/kg to about 10 mg/kg, about 10 mg/kg to about 100 mg/kg, from about 100 mg/kg to about 500 mg/kg or from about 500 mg/kg to about 1000 mg/kg. The amount may be 10 mg/kg. The pharmaceutically acceptable form of the composition includes a pharmaceutically acceptable carrier.

The preparation of therapeutic compositions which contain an active component is well understood in the art. Typically, such compositions are prepared as an aerosol of the polypeptide delivered to the nasopharynx or as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

An active component can be formulated into the therapeutic composition as neutralized pharmaceutically acceptable salt forms. Pharmaceutically acceptable salts include the acid addition salts (formed with the free amino groups of the polypeptide) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The component or components of a therapeutic composition provided herein may be introduced parenterally, transmucosally, e.g., orally, nasally, pulmonarily, or rectally, or transdermally. Preferably, administration is parenteral, e.g., via intravenous injection, and also including, but is not limited to, intra-arteriole, intramuscular, intradermal, subcutaneous, intraperitoneal, intraventricular, and intracranial administration. The term “unit dose” when used in reference to a therapeutic composition provided herein refers to physically discrete units suitable as unitary dosage for humans, each unit containing a predetermined quantity of active material calculated to produce the desired therapeutic effect in association with the required diluent; i.e., carrier, or vehicle.

In another embodiment, the active compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

In yet another embodiment, the therapeutic compound can be delivered in a controlled release system. For example, the protein may be administered using intravenous infusion, an implantable osmotic pump, a transdermal patch, liposomes, or other modes of administration. In one embodiment, a pump may be used (see Langer, supra; Sefton (1987) CRC Crit. Ref Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas (1983) J. Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., the brain or a tumor, thus requiring only a fraction of the systemic dose (see, e.g., Goodson, in Medical Applications of Controlled Release, supra, vol. 2, pp. 115-138 (1984)). Other controlled release systems are discussed in the review by Langer (1990) Science 249:1527-1533.

A subject in whom administration of an active component as set forth above is an effective therapeutic regimen for an infection by an infectious disease organism is preferably a human, but can be any animal. Thus, as can be readily appreciated by one of ordinary skill in the art, the methods and pharmaceutical compositions provided herein are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., i.e., for veterinary medical use.

In the therapeutic methods and compositions provided herein, a therapeutically effective dosage of the active component is provided. A therapeutically effective dosage can be determined by the ordinary skilled medical worker based on patient characteristics (age, weight, sex, condition, complications, other diseases, etc.), as is well known in the art. Furthermore, as further routine studies are conducted, more specific information will emerge regarding appropriate dosage levels for treatment of various conditions in various patients, and the ordinary skilled worker, considering the therapeutic context, age and general health of the recipient, is able to ascertain proper dosing. Generally, for intravenous injection or infusion, dosage may be lower than for intraperitoneal, intramuscular, or other route of administration. The dosing schedule may vary, depending on the circulation half-life, and the formulation used. The compositions are administered in a manner compatible with the dosage formulation in the therapeutically effective amount. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

Also contemplated are dry powder formulations comprising at least one protein provided herein and another therapeutically effective drug, such as an antibiotic or a chemotherapeutic agent.

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include the component or components (or chemically modified forms thereof) and inert ingredients which allow for protection against the stomach environment, and release of the biologically active material in the intestine.

Also specifically contemplated are oral dosage forms of the above derivatized component or components. The component or components may be chemically modified so that oral delivery of the derivative is efficacious. Generally, the chemical modification contemplated is the attachment of at least one moiety to the component molecule itself, where the moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis (1981) “Soluble Polymer-Enzyme Abducts” In: Enzymes as Drugs, Hocenberg and Roberts, eds., Wiley-Interscience, New York, N.Y., pp. 367-383; Newmark, et al. (1982) J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the protein (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The peptide therapeutic can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

One may dilute or increase the volume of the therapeutic with an inert material. These diluents could include carbohydrates, especially mannitol, a-lactose, anhydrous lactose, cellulose, sucrose, modified dextran and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants. Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives which potentially enhance uptake of the protein (or derivative) are for instance the fatty acids oleic acid, linoleic acid and linolenic acid.

In one embodiment, the method comprises the use of viruses for administering any of the various target molecules associated with Perforin-2 expression, function or activity provided herein or any of the various agents provided herein which modulate the function or activity of one or more target molecules associated with Perforin-2 expression, function or activity to a subject. Administration can be by the use of viruses that express any of the target molecules or agents provided herein, such as recombinant retroviruses, recombinant adeno-associated viruses, recombinant adenoviruses, and recombinant Herpes simplex viruses (see, for example, Mulligan, Science 260:926 (1993), Rosenberg et al., Science 242:1575 (1988), LaSalle et al., Science 259:988 (1993), Wolff et al., Science 247:1465 (1990), Breakfield and Deluca, The New Biologist 3:203 (1991)).

A gene encoding any of the various target molecules or agents provided herein can be delivered using recombinant viral vectors, including for example, adenoviral vectors (e.g., Kass-Eisler et al., Proc. Nat'l Acad. Sci. USA 90:11498 (1993), Kolls et al., Proc. Nat'l Acad. Sci. USA 91:215 (1994), Li et al., Hum. Gene Ther. 4:403 (1993), Vincent et al., Nat. Genet. 5:130 (1993), and Zabner et al., Cell 75:207 (1993)), adenovirus-associated viral vectors (Flotte et al., Proc. Nat'l Acad. Sci. USA 90:10613 (1993)), alphaviruses such as Semliki Forest Virus and Sindbis Virus (Hertz and Huang, J. Vir. 66:857 (1992), Raju and Huang, J. Vir. 65:2501 (1991), and Xiong et al., Science 243:1188 (1989)), herpes viral vectors (e.g., U.S. Pat. Nos. 4,769,331, 4,859,587, 5,288,641 and 5,328,688), parvovirus vectors (Koering et al., Hum. Gene Therap. 5:457 (1994)), pox virus vectors (Ozaki et al., Biochem. Biophys. Res. Comm. 193:653 (1993), Panicali and Paoletti, Proc. Nat'l Acad. Sci. USA 79:4927 (1982)), pox viruses, such as canary pox virus or vaccinia virus (Fisher-Hoch et al., Proc. Nat'l Acad. Sci. USA 86:317 (1989), and Flexner et al., Ann. N.Y. Acad. Sci. 569:86 (1989)), and retroviruses (e.g., Baba et al., J. Neurosurg 79:729 (1993), Ram et al., Cancer Res. 53:83 (1993), Takamiya et al., J. Neurosci. Res 33:493 (1992), Vile and Hart, Cancer Res. 53:962 (1993), Vile and Hart, Cancer Res. 53:3860 (1993), and Anderson et al., U.S. Pat. No. 5,399,346). Within various embodiments, either the viral vector itself, or a viral particle, which contains the viral vector may be utilized in the methods described below.

As an illustration of one system, adenovirus, a double-stranded DNA virus, is a well-characterized gene transfer vector for delivery of a heterologous nucleic acid molecule (for a review, see Becker et al., Meth. Cell Biol. 43:161 (1994); Douglas and Curiel, Science & Medicine 4:44 (1997)). The adenovirus system offers several advantages including: (i) the ability to accommodate relatively large DNA inserts, (ii) the ability to be grown to high-titer, (iii) the ability to infect a broad range of mammalian cell types, and (iv) the ability to be used with many different promoters including ubiquitous, tissue specific, and regulatable promoters. In addition, adenoviruses can be administered by intravenous injection, because the viruses are stable in the bloodstream.

Using adenovirus vectors where portions of the adenovirus genome are deleted, inserts are incorporated into the viral DNA by direct ligation or by homologous recombination with a co-transfected plasmid. In an exemplary system, the essential E1 gene is deleted from the viral vector, and the virus will not replicate unless the E1 gene is provided by the host cell. When intravenously administered to intact animals, adenovirus primarily targets the liver. Although an adenoviral delivery system with an E1 gene deletion cannot replicate in the host cells, the host's tissue will express and process an encoded heterologous protein. Host cells will also secrete the heterologous protein if the corresponding gene includes a secretory signal sequence. Secreted proteins will enter the circulation from tissue that expresses the heterologous gene (e.g., the highly vascularized liver).

Moreover, adenoviral vectors containing various deletions of viral genes can be used to reduce or eliminate immune responses to the vector. Such adenoviruses are E1-deleted, and in addition, contain deletions of E2A or E4 (Lusky et al., J. Virol. 72:2022 (1998); Raper et al., Human Gene Therapy 9:671 (1998)). The deletion of E2b has also been reported to reduce immune responses (Amalfitano et al., J. Virol. 72:926 (1998)). By deleting the entire adenovirus genome, very large inserts of heterologous DNA can be accommodated. Generation of so called “gutless” adenoviruses, where all viral genes are deleted, are particularly advantageous for insertion of large inserts of heterologous DNA (for a review, see Yeh. and Perricaudet, FASEB J. 11:615 (1997)).

High titer stocks of recombinant viruses capable of expressing a therapeutic gene can be obtained from infected mammalian cells using standard methods. For example, recombinant herpes simplex virus can be prepared in Vero cells, as described by Brandt et al., J. Gen. Virol. 72:2043 (1991), Herold et al., J. Gen. Virol. 75:1211 (1994), Visalli and Brandt, Virology 185:419 (1991), Grau et al., Invest. Ophthalmol. Vis. Sci. 30:2474 (1989), Brandt et al., J. Virol. Meth. 36:209 (1992), and by Brown and MacLean (eds.), HSV Virus Protocols (Humana Press 1997).

When the subject treated with a recombinant virus is a human, then the therapy is preferably somatic cell gene therapy. That is, the preferred treatment of a human with a recombinant virus does not entail introducing into cells a nucleic acid molecule that can form part of a human germ line and be passed onto successive generations (i.e., human germ line gene therapy).

Infectious Organisms

As used herein, “infectious organisms” can include, but are not limited to, for example, bacteria, viruses, fungi, parasites and protozoa.

Particularly preferred bacteria causing serious human diseases are the Gram positive organisms: Staphylococcus aureus, Methicillin-resistant Staphylococcus aureus (MRSA), Staphylococcus epidermidis, Enterococcus faecalis and E. faecium, Streptococcus pneumoniae and the Gram negative organisms: Pseudomonas aeruginosa, Burkholdia cepacia, Xanthomonas maltophila, Escherichia coli, Enteropathogenic E. coli (EPEC), Enterobacter spp, Klebsiella pneumonia, Chlamydia spp., including Chlamydia trachomatis, and Salmonella spp.

In another preferred embodiment, the bacteria are Gram negative bacteria. Examples, comprise: Pseudomonas aeruginosa; Burkholdia cepacia; Xanthomonas maltophila; Escherichia coli; Enterobacter spp.; Klebsiella pneumoniae; Salmonella spp.

The present invention also provides methods for treating diseases include infections by Mycobacterium spp., Mycobacterium tuberculosis, Entamoeba histolytica; Pneumocystis carinii, Trypanosoma cruzi, Trypanosoma brucei, Leishmania mexicana, Clostridium histolyticum, Staphylococcus aureus, foot-and-mouth disease virus and Crithidia fasciculata; as well as in osteoporosis, autoimmunity, schistosomiasis, malaria, tumor metastasis, metachromatic leukodystrophy, muscular dystrophy and amytrophy.

Other examples include veterinary and human pathogenic protozoa, intracellular active parasites of the phylum Apicomplexa or Sarcomastigophora, Trypanosoma, Plasmodia, Leishmania, Babesia and Theileria, Cryptosporidia, Sacrocystida, Amoeba, Coccidia and Trichomonadia. These compounds are also suitable for the treatment of Malaria tropica, caused by, for example, Plasmodium falciparum, Malaria tertiana, caused by Plasmodium vivax or Plasmodium ovale and for the treatment of Malaria quartana, caused by Plasmodium malariae. They are also suitable for the treatment of Toxoplasmosis, caused by Toxoplasma gondii, Coccidiosis, caused for instance by Isospora belli, intestinal Sarcosporidiosis, caused by Sarcocystis suihominis, dysentery caused by Entamoeba histolytica, Cryptosporidiosis, caused by Cryptosporidium parvum, Chagas' disease, caused by Trypanosoma cruzi, sleeping sickness, caused by Trypanosoma brucei rhodesiense or gambiense, the cutaneous and visceral as well as other forms of Leishmaniosis. They are also suitable for the treatment of animals infected by veterinary pathogenic protozoa, like Theileria parva, the pathogen causing bovine East coast fever, Trypanosoma congolense congolense or Trypanosoma vivax vivax, Trypanosoma brucei brucei, pathogens causing Nagana cattle disease in Africa, Trypanosoma brucei evansi causing Surra, Babesia bigemina, the pathogen causing Texas fever in cattle and buffalos, Babesia bovis, the pathogen causing European bovine Babesiosis as well as Babesiosis in dogs, cats and sheep, Sarcocystis ovicanis and ovifelis pathogens causing Sarcocystiosis in sheep, cattle and pigs, Cryptosporidia, pathogens causing Cryptosporidioses in cattle and birds, Eimeria and Isospora species, pathogens causing Coccidiosis in rabbits, cattle, sheep, goats, pigs and birds, especially in chickens and turkeys. Rickettsia comprise species such as Rickettsia felis, Rickettsia prowazekii, Rickettsia rickettsii, Rickettsia typhi, Rickettsia conorii, Rickettsia africae and cause diseases such as typhus, rickettsialpox, Boutonneuse fever, African Tick Bite Fever, Rocky Mountain spotted fever, Australian Tick Typhus, Flinders Island Spotted Fever and Queensland Tick Typhus. In the treatment of these diseases, the compounds of the present invention may be combined with other agents.

Particularly preferred fungi causing or associated with human diseases according to the present invention include (but not restricted to) Candida albicans, Histoplasma neoformans, Coccidioides immitis and Penicillium marneffei.

Transgenic Animals

The functional activity of Perforin-2 can be evaluated transgenically. In this respect, a transgenic mouse model can be used. The Perforin-2 gene can be used in complementation studies employing a transgenic mouse. Transgenic vectors, including viral vectors, or cosmid clones (or phage clones) corresponding to the wild type locus of a candidate gene, can be constructed using the isolated Perforin-2 gene. Cosmids may be introduced into transgenic mice using published procedures [Jaenisch (1988) Science 240:1468-1474]. In a genetic sense, the transgene acts as a suppressor mutation.

Alternatively, a transgenic animal model can be prepared in which expression of the Perforin-2 gene is disrupted. One standard method to evaluate the phenotypic effect of a gene product is to employ knockout technology to delete or inactivate the gene. As used herein, the terms “disruption” or “knockout” refer to a partial or complete inhibition of the expression of at least a portion of a protein encoded by a DNA sequence in a cell. By “partial” inhibition or inactivation is meant that gene expression is decreased by at least 5%, 10%, 20%, 30%, 40%, 50%, 60%, 70%, 75%, 80%, 85%, 90%, 95% or more as compared to gene expression when the gene is not disrupted (i.e. wild-type). By “complete inhibition” is meant that no functional protein is expressed (i.e. 100% inhibition of gene expression).

A knockout includes both the heterozygous mutant and the homozygous mutant. As used herein, a “heterozygous” gene disruption or knockout comprises one defective allele and one wild-type allele. A “homozygous” gene disruption or knockout comprises two defective alleles. For example, a homozygous knockout mouse comprises disruption of both alleles of a gene and a heterozygous knockout mouse comprises disruption of one allele of a gene. As used herein, in reference to a gene or knockout, “wild-type” refers to the native, non-mutated or non-disrupted form of a gene.

Provided herein are transgenic animals in which the Perforin-2 gene has been disrupted. In one embodiment, a transgenic mouse which comprises a disruption of a gene encoding a Perforin-2 protein is provided. In some embodiments, the disruption of the Perforin-2 gene can be heterozygous or homozygous.

In a specific embodiment, the homozygous disruption inactivates the Perforin-2 gene and inhibits the expression of a functional Perforin-2 protein in the transgenic mouse.

In another embodiment, the gene disruption partially inactivates the Perforin-2 gene. In a specific embodiment, the gene disruption is heterozygous.

In such embodiments, a transgenic Perforin-2 knockout mouse exhibits an increased susceptibility to infection by intracellular pathogens as compared to a wild-type mouse.

Also provided herein is an organ, a tissue, a cell, or a cell-line derived from a transgenic mouse comprising a Perforin-2 gene disruption.

Alternatively, recombinant techniques can be used to introduce mutations, such as nonsense and amber mutations, or mutations that lead to expression of an inactive protein. In another embodiment, Perforin-2 genes can be tested by examining their phenotypic effects when expressed in antisense orientation in wild-type animals. In this approach, expression of the wild-type allele is suppressed, which leads to a mutant phenotype. RNA×RNA duplex formation (antisense-sense) prevents normal handling of mRNA, resulting in partial or complete elimination of wild-type gene effect. This technique has been used to inhibit TK synthesis in tissue culture and to produce phenotypes of the Kruppel mutation in Drosophila, and the Shiverer mutation in mice [Izant et al. Cell (1984) 36:1007-1015; Green et al. (1986) Annu. Rev. Biochem. 55:569-597; Katsuki et al. (1988) Science 241:593-595]. An important advantage of this approach is that only a small portion of the gene need be expressed for effective inhibition of expression of the entire cognate mRNA. The antisense transgene will be placed under control of its own promoter or another promoter expressed in the correct cell type, and placed upstream of the SV40 polyA site. This transgene will be used to make transgenic mice, or by using gene knockout technology.

In this disclosure there is described only the preferred embodiments of the invention and but a few examples of its versatility. It is to be understood that the invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances and procedures described herein. Such equivalents are considered to be within the scope of this invention.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

Non-limiting examples of methods and compositions disclosed herein are as follows:

1. A method of modulating function, activity or expression of Perforin-2 (P2) in vitro or in vivo comprising: contacting a cell in vitro or administering to a patient, an effective amount of at least one agent which modulates function, activity or expression of one or more molecules associated with P2 expression, function or activity; and, modulating the function or expression of P2.
2. The method of embodiment 1, wherein the one or more molecules associated with P2 function, activity or expression comprise: src, ubiquitin conjugating enzyme E2M, GAPDH, P21RAS/gap1m, Galectin 3, ubiquitin C (UCHL1), proteasomes, or fragments thereof.
3. The method of embodiment 2, wherein the P2 expression, function or activity is upregulated by administration of the at least one agent which upregulates the function, activity or expression of the one or more molecules associated with P2 function, expression or activity.
4. The method of embodiment 2, wherein the P2 expression, function or activity is downregulated by administration of the at least one agent which downregulates the function, activity or expression of the one or more molecules associated with P2 function, expression or activity.
5. The method of embodiment 2, wherein the P2 expression, function or activity is upregulated by administration of at least one agent which independently upregulates or downregulates the function, activity or expression of at least two molecules associated with P2 function, expression or activity.
6. The method of embodiment 2, wherein the P2 expression, function or activity is downregulated by administration of at least one agent which independently upregulates or downregulates the function, activity or expression of at least two molecules associated with P2 function, expression or activity.
7. The method of embodiment 2, wherein the P2 expression, function or activity is upregulated by administration of a combination of at least two agents which independently upregulate or downregulate the function, activity or expression of at least two molecules associated with P2 function, expression or activity.
8. The method of embodiment 2, wherein the P2 expression, function or activity is downregulated by administration of a combination of at least two agents which independently upregulate or downregulate the function, activity or expression of at least two molecules associated with P2 function, expression or activity.
9. The method of embodiment 2, comprising an optional step of administering an agent which directly modulates expression, function or activity of the P2 molecule.
10. The method of embodiment 9, wherein the molecule inhibits transcription or translation of P2.
11. The method of embodiment 1, wherein an agent comprises: a small molecule, protein, peptide, polypeptide, modified peptides, modified oligonucleotides, oligonucleotide, polynucleotide, synthetic molecule, natural molecule, organic or inorganic molecule, or combinations thereof.
12. A method of identifying a candidate therapeutic agent comprising: contacting a cell expressing one or more target molecules comprising: src, ubiquitin conjugating enzyme E2M, GAPDH, P21RAS/gap1m, Galectin 3, ubiquitin C (UCHL1), proteasomes, or fragments thereof; measuring the expression, function or activity of the molecules; comparing the expression, function or activity of the molecules with a control; and, identifying a candidate therapeutic agent.
13. The method of embodiment 12, wherein the candidate therapeutic agent modulates the expression, function or activity of one or more of the target molecules.
14. The method of embodiment 12, wherein the candidate therapeutic agent modulates the expression, function or activity of a plurality of target molecules.
15. The method of embodiment 12, wherein the candidate therapeutic agent upregulates the expression, function or activity of one or more of the target molecules.
16. The method of embodiment 12, wherein the candidate therapeutic agent downregulates the expression, function or activity of one or more of the target molecules.
17. The method of embodiment 12, wherein the modulation of the expression, function or activity of one or more target molecules modulates the expression, function or activity of Perforin-2 (P2) molecules.
18. The method of embodiment 17, wherein the upregulation of the expression, function or activity of one or more target molecules upregulates the expression, function or activity of Perforin-2 (P2) molecules.
19. The method of embodiment 17, wherein the downregulation of the expression, function or activity of one or more target molecules downregulates the expression, function or activity of Perforin-2 (P2) molecules.
20. The method of embodiment 12, wherein the target molecules are polynucleotides or expressed products thereof.
21. A method of identifying a candidate therapeutic agent comprising: contacting an assay surface with one or more target molecules comprising src, ubiquitin conjugating enzyme E2M, GAPDH, P21RAS/gap1m, Galectin 3, ubiquitin C (UCHL1), proteasomes, fragments or associated molecules thereof; contacting the target molecules with one or more candidate therapeutic agents and identifying the agents which bind or hybridize to one or more target molecules or associated molecules thereof.
22. The method of embodiment 21, wherein the identified candidate therapeutic agents are assayed for modulation of expression, function or activity of Perforin-2 molecules.
23. The method of embodiment 22, wherein an identified candidate therapeutic agent upregulates the expression, function, or activity of P2 molecules.
24. The method of embodiment 22, wherein an identified candidate therapeutic agent downregulates the expression, function, or activity of P2 molecules.
25. The method of embodiment 22, wherein the assays for assaying the expression, function or activity of P2 molecules comprise: cellular assays, immuno-assays, yeast hybrid system assays, hybridization assays, nucleic acid based assays, high-throughput screening assays or combinations thereof.
26. The method of embodiment 22, wherein the identified candidate agents are assayed for inhibition of replication, inhibition of growth, or death of an infectious organism.
27. The method of embodiment 26, wherein the infectious organism is an intracellular or extracellular bacterium.
28. A method of treating a patient suffering from an infectious disease organism comprising, administering to the patient a therapeutically effective amount of an agent identified by the methods of embodiment 1 or embodiment 21.
29. A compound identified by the method of embodiment 1 or embodiment 21.
30. A pharmaceutical composition comprising a compound of embodiment 29.
31. A method of identifying individuals at risk from pathogenic infections comprising: obtaining a patient sample, assaying for one or more molecules comprising: src, ubiquitin conjugating enzyme E2M, GAPDH, P21RAS/gap1m, Galectin 3, ubiquitin C (UCHL1), proteasomes, Perforin-2, fragments or associated molecules thereof; and, comparing the expression, function or activity with a normal control.
32. The method of embodiment 31, wherein an individual identified as having downregulated expression levels, decreased activity or functions as compared to the control, would be prognostic for risk of infection.
33. A method to identify at least one agent that modulates expression, function or activity of Perforin-2, the method comprising: (a) contacting a cell expressing one or more target molecules associated with Perforin-2 expression, function or activity with the at least one agent; (b) measuring the expression, function or activity of said one or more target molecules associated with Perforin-2 expression, function or activity; and (c) comparing the expression, function or activity of said one or more target molecules with a control, wherein contact with the at least one agent modulates the expression, function or activity of said one or more target molecules thereby identifying said agent that modulates expression, function or activity of Perforin-2.
34. The method of embodiment 33, wherein the one or more target molecules associated with Perforin-2 expression, function or activity comprise: src, ubiquitin conjugating enzyme E2M (Ubc12), GAPDH, P21RAS/gap1m (RASA2), Galectin 3, ubiquitin C (UCHL1), proteasomes, vps34, ATG5, ATG7, ATG9L1, ATG14L, ATG16L, LC3, Rab5, or fragments thereof.
35. The method of any one of embodiments 33-34, wherein the at least one agent upregulates the expression, function or activity of said one or more target molecules associated with Perforin-2 expression, function or activity.
36. The method of embodiment 35, wherein the upregulation of the expression, function or activity of the one or more target molecules upregulates the expression, function or activity of Perforin-2.
37. The method of any one of embodiments 33-34, wherein the at least one agent downregulates the expression, function or activity of said one or more target molecules associated with Perforin-2 expression, function or activity.
38. The method of embodiment 37, wherein the downregulation of the expression, function or activity of the one or more target molecules downregulates the expression, function or activity of Perforin-2.
39. The method of any one of embodiments 33-34, wherein the Perforin-2 expression, function or activity is upregulated by at least one agent which independently upregulates or downregulates the expression, function or activity of at least two target molecules associated with Perforin-2 expression, function or activity.
40. The method of any one of embodiments 33-34, wherein the Perforin-2 expression, function or activity is downregulated by at least one agent which independently upregulates or downregulates the function, activity or expression of at least two target molecules associated with Perforin-2 expression, function or activity.
41. The method of any one of embodiments 33-34, wherein the Perforin-2 expression, function or activity is upregulated by a combination of at least two agents which independently upregulate or downregulate the expression, function or activity of at least two target molecules associated with Perforin-2 expression, function or activity.
42. The method of any one of embodiments 33-34, wherein the Perforin-2 expression, function or activity is downregulated by a combination of at least two agents which independently upregulate or downregulate the expression, function or activity of at least two target molecules associated with Perforin-2 expression, function or activity.
43. The method of any one of embodiments 33-42, wherein the agent comprises: a small molecule, protein, peptide, polypeptide, modified peptides, modified oligonucleotides, oligonucleotide, polynucleotide, synthetic molecule, natural molecule, organic or inorganic molecule, or combinations thereof.
44. The method of embodiment 33, wherein the one or more target molecules associated with Perforin-2 expression, function or activity is from an infectious organism.
45. The method of embodiment 44, wherein said infectious organism is a bacterium.
46. The method of any one of embodiments 44-45, wherein the at least one agent downregulates the expression, function or activity of the one or more target molecules.
47. The method of any one of embodiments 44-45, wherein the at least one agent upregulates the expression, function or activity of the one or more target molecules.
48. The method of embodiment 46, wherein the downregulation of the expression, function or activity of the one or more target molecules upregulates the expression, function or activity of Perforin-2.
49. The method of embodiment 47, wherein the upregulation of the expression, function or activity of the one or more target molecules downregulates the expression, function or activity of Perforin-2.
50. The method of any one of embodiments 44-45, wherein the Perforin-2 expression, function or activity is upregulated by at least one agent which independently upregulates or downregulates the expression, function or activity of at least two target molecules associated with Perforin-2 expression, function or activity.
51. The method of any one of embodiments 44-45, wherein the Perforin-2 expression, function or activity is downregulated by at least one agent which independently upregulates or downregulates the expression, function or activity of at least two target molecules associated with Perforin-2 expression, function or activity.
52. The method of embodiment 44, wherein the bacterium is Salmonella typhimurium or Escherichia coli.
53. The method of any one of embodiments 44-45, wherein the one or more target molecules associated with Perforin-2 expression, function or activity comprise: PhoP or deamidase.
54. A method of identifying a candidate agent that modulates expression, function or activity of Perforin-2 comprising: (a) contacting an assay surface with one or more target molecules comprising src, ubiquitin conjugating enzyme E2M, GAPDH, P21RAS/gap1m, Galectin 3, ubiquitin C (UCHL1), proteasomes, vps34, ATG5, ATG7, ATG9L1, ATG14L, ATG16L, LC3, Rab5, PhoP, deamidase, fragments or associated molecules thereof; (b) contacting the target molecules with one or more candidate agents and identifying the agents which bind or hybridize to one or more target molecules or associated molecules thereof; and (c) assaying said one or more candidate agents for modulation of expression, function or activity of Perforin-2, thereby identifying said candidate agent.
55. The method of embodiment 54, wherein an identified candidate agent upregulates the expression, function, or activity of Perforin-2.
56. The method of embodiment 54, wherein an identified candidate agent downregulates the expression, function, or activity of Perforin-2.
57. The method of embodiment 54, wherein the assays for assaying the expression, function or activity of Perforin-2 molecules comprise: cellular assays, immuno-assays, yeast hybrid system assays, hybridization assays, nucleic acid based assays, high-throughput screening assays or combinations thereof.
58. The method of embodiment 54, wherein the identified candidate agents are assayed for inhibition of replication, inhibition of growth, or death of an infectious organism.
59. The method of embodiment 58, wherein the infectious organism is an intracellular or extracellular bacterium.
60. A method of identifying individuals at risk from pathogenic infections comprising: obtaining a patient sample, assaying for one or more molecules comprising: src, ubiquitin conjugating enzyme E2M, GAPDH, P21RAS/gap1m, Galectin 3, ubiquitin C (UCHL1), proteasomes, Perforin-2, vps34, ATG5, ATG7, ATG9L1, ATG14L, ATG16L, LC3, Rab5, fragments or associated molecules thereof; and, comparing the expression, function or activity with a normal control.
61. The method of embodiment 60, wherein an individual identified as having downregulated expression levels, decreased activity or functions as compared to the control, would be prognostic for risk of infection.
62. A transgenic mouse which comprises a disruption of a gene encoding a Perforin-2 protein.
63. The transgenic mouse of embodiment 62, wherein said disruption comprises a heterozygous or homozygous disruption of said gene encoding a Perforin-2 protein.
64. The transgenic mouse of embodiment 63, wherein said disruption comprises a homozygous disruption, wherein said homozygous disruption inactivates said gene and inhibits the expression of a functional Perforin-2 protein in said transgenic mouse.
65. The transgenic mouse of any one of embodiments 62-64, wherein said transgenic mouse exhibits an increased susceptibility to infection by intracellular pathogens as compared to a wild-type mouse.
66. An organ, a tissue, a cell, or a cell-line derived from the transgenic mouse of any one of embodiments 62-64.

### EXAMPLES

### Example 1

### A Pore-Forming Protein in Macrophages and Microglia Kills Pathogenic Intracellular Bacteria

The example shows that Mpeg1 encodes a novel pore forming protein, designated Perforin-2 (P-2), forming transmembrane pores by polymerization. P-2 in macrophages has potent intracellular killing activity for pathogenic bacteria including methicillin resistant Staphylococcus aureus (MRSA), Mycobacterium avium (Ma), Mycobacterium smegmatis (Msm), Salmonella typhimurium (St) and E. coli. Moreover, P-2 enabled the bactericidal activity of reactive oxygen species (ROS) and nitric oxide (NO).

### Materials and Methods

Plasmid Constructs:

The complete coding region of murine Mpeg-1 cDNA was constructed from several EST clones and inserted into the pEGFP-N3 plasmid (Clontech). Monomeric RFP was cloned in place of GFP for use in some experiments.

Cell Lines and Primary Cells:

HEK-293 (ATCC), RAW264.7 (ATCC) and cell lines were maintained in IMDM supplemented with 10% FBS. Primary macrophages were obtained from the peritoneum or bone marrow. Thioglycollate-elicited peritoneal macrophages: 1.5 ml of a 3% thioglycollate solution was injected i.p. into C57/B6 mice. 4 days later, peritoneal cells were harvested and purified by adherence for macrophage cells. Bone-marrow derived macrophages: bone marrow was flushed from the long bones of C57BL/6 mice. Red blood cells were lysed with ACK buffer and the cell pellet resuspended (10⁶ cells/ml) in complete medium containing 20 ng/ml granulocyte macrophage colony stimulating factor (GM-CSF) (Peprotech, Rocky Hill, N.J., USA). On day 4, the non-adherent cells were harvested and replated in fresh complete medium. Fresh medium was added every 3 days until cells were ready for experiments (usually between day 7 and 10).

Negative Staining Electron Microscopy:

Membranes were isolated from P2GFP-transfected 293 cells by N2-cavitation and differential centrifugation. Membranes were resuspended in a small volume of neutral Tris-buffered saline, treated with 100 μg/ml trypsin for 1 h at 37° C., washed and negatively stained with 5% neutral Na-phosphotungstic acid for 30 seconds. Images were taken at 52,000 fold initial magnification on a Phillips CM10 transmission electron microscope.

Gentamicin Protection Assay:

S. Typhimurium strain LT2Z, Mycobacterium avium, Mycobacterium smegmatis (ATCC), methicllin resistant Staphylococcus aureus and K12 E. coli were grown from glycerol stocks at 37° C. with shaking for 16-18 hr in Luria broth (LB) (S. typhimurium, S. aureus, and E. coli) or Middlebrook 7H9 broth (Mycobacteria) prior to infection. For Salmonella, the culture was then diluted 1:33 in LB and grown for another 3 hours to induce invasive phenotype. Macrophages or microglial cells were plated (5×10⁵ cells/well of a 12 well plate) and stimulated overnight with LPS (1 ng/ml) and IFN-γ (100 U/ml). Cells were infected the next day at indicated MOI for 30 minutes (S. typhimurium), or 1 hour (all other bacteria) in 37° C., 5% CO₂ incubator. Cells were washed twice with PBS and fresh medium containing 50 μg/ml gentamicin was added. After 2 hours the concentration of gentamicin was lowered to 5 μg/ml. At the indicated timepoints after adding gentamicin, the cells were washed with PBS, lysed using 0.1% Triton-X in water, diluted and plated in triplicate on agar plates and CFU determined.

RT-PCR:

RNA was extracted from cells according to RNeasy (Qiagen) instructions. 1 μg of RNA was converted to cDNA using QuantiTect Reverse Transcription Kit (Qiagen) RT-PCR was performed using TAQMAN® Gene Expression Assays (Applied Biosystems) for murine Mpeg-1 and GAPDH, as a housekeeping control gene. All assays were performed on Applied Biosystems 7300 PCR platform.

Antibodies:

Rabbit anti-Mpeg1 and anti-GFP polyclonal antibodies were obtained from Abcam and used for western blot analysis. Rabbit anti-P2 (cytoplasmic domain) antiserum was produced and obtained by 21st Century.

RNA Interference:

Three P2-specific chemically synthesized 19-nucleotide siRNA duplexes were obtained from Sigma. Two siRNAs were complementary to the 3′ UTR of P2 and the third to the coding region. The sequences were as follows: CCACCUCACUUUCUAUCAA (SEQ ID NO: 1), GAGUAUUCUAGGAAACUUU (SEQ ID NO: 2), and CAAUCAAGCUCUUGUGCAC (SEQ ID NO: 3). Transfection of siRNA into macrophage and microglial cells was carried out using Amaxa Nucleofector System (Lonza) according to manufacturer's instructions. All transfections were carried out using 4×10⁶ cells and a final concentration of 1 μM siRNA (P2-specific siRNAs were pooled). Immediately after transfection cells were plated in antibiotic-free IMDM containing 10% FBS.

Confocal Microscopy:

For live cell imaging, RAW cells were nucleofected with P2GFP and stimulated overnight with LPS (1 ng/ml) and IFN-γ (100 U/ml) in glass bottom dishes with No. 1.5 coverglass (MatTek Corp.). Cells were washed once with PBS and organelles were labeled. For endoplasmic reticulum (ER) labeling, ER-TRACKER™ Blue-White DPX (Invitrogen) was used at a working concentration of 1 μM for 30 minutes at 37° C. For all other stains, transfected cells were fixed with 3% paraformaldehyde (PFA) for 15 min at room temperature, permeabilized with 0.5% saponin, blocked with 10% normal goat serum and incubated with primary and secondary antibodies. Anti-CD107a (LAMP-1) (BD Pharmingen), anti-CD11b (BD Pharmingen), anti-golgin97 (Invitrogen), anti-EEA1 (Calbiochem), anti-GM130 (BD biosciences), and Hoechst 33258 (Invitrogen) were used to identify cellular organelles. Secondary antibodies were all raised in goats. Images were taken on a Leica SP5 inverted confocal microscope with a motorized stage and analyzed using Leica application suite advanced fluorescence software.

Results and Discussion

Macrophages constitutively transcribe Mpeg1 mRNA which predicts a protein with a MACPF domain typically found in cytolytic pore-forming proteins. The founding members of the MACPF domain are the pore-forming complement component C9 of the membrane attack complex, killing extracellular bacteria, and Perforin-1, the pore-forming molecule of T and NK lymphocytes killing virus-infected cells and tumor cells. Pore formation is achieved by polymerization of the MACPF domain which mediates conformational changes resulting in membrane insertion of four amphipathic β-strands leading to perforation and target cell death. In this example it is shown that Mpeg1 encodes a novel pore-forming protein, designated Perforin-2 (P-2), assembling transmembrane pores by polymerization. P-2 in macrophages has potent killing activity for intracellular pathogenic bacteria including Mycobacterium avium, (M. avium), M. smegmatis, Salmonella typhimurium (S. typhimurium), Escherichia coli (E. coli) and clinical isolates of Methicillin resistant Staphylococcus aureus (MRSA). Importantly, P-2 also contributes to the antimicrobial activity of ROS and NO.

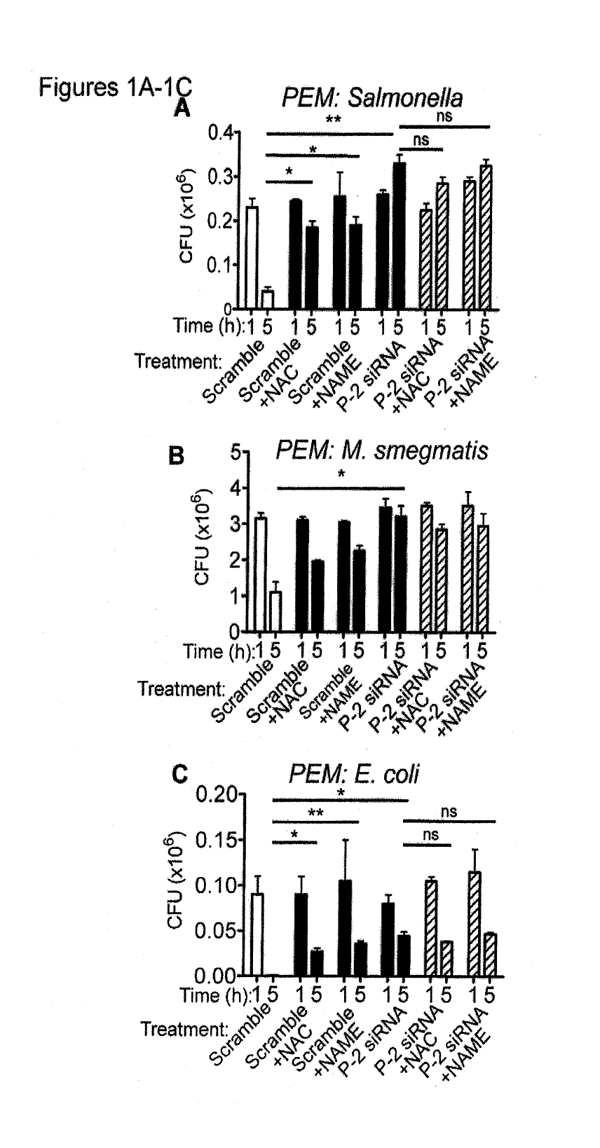

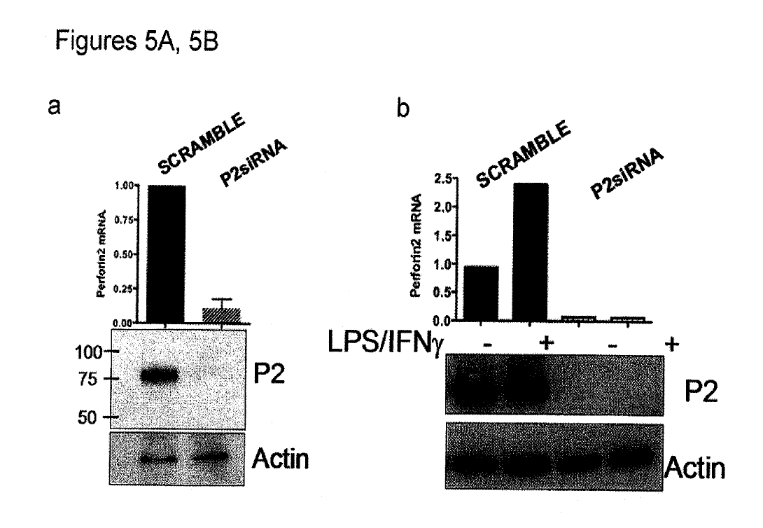

The current understanding of bactericidal responses in macrophages is that they are mediated by oxidative mechanisms, such as ROS and NO within the phagosome, and by phagosome-lysosome fusion. To assess the role of P-2 in comparison to ROS and NO in intracellular killing of bacteria, each of the bactericidal effectors was blocked individually or in combination with P-2, and intracellular bacterial survival in peritoneal macrophages (PEM) measured between 1 and 5 hours post-infection utilizing the gentamycin protection assay (FIGS. 1A-1C). Gentamycin is membrane impermeable and present only in the extracellular medium. Inhibition of ROS, NO or P-2 individually had different effects on different bacteria. Using S. typhimurium, ˜90% bacteria present intracellularly in PEM at 1 h were dead at 5 h in the absence of inhibitors (˜10% survival, FIG. 1 A). Blockade of ROS with the antioxidant and ROS scavenger N-Acetylcysteine (NAC) allowed ˜80% survival of Salmonellae at 5 h compared to 1 h levels, indicating that only 20% are killed without the help of ROS. Blockade of NO with the nitric oxide synthase inhibitor N^{G}-nitro-L-arginine-methyl ester hydrochloride (L-NAME) has similar effects as NAC. P-2 knockdown by transfection with P-2-specific siRNAs (data for efficiency of knock down in FIGS. 5A, 5B) on the other hand eliminated all killing and allowed intracellular replication of Salmonellae (FIG. 1 A) even though ROS and NO were not blocked. Importantly, combination of NAC or L-NAME with P-2 knockdown did not further increase intracellular replication above that provided by P-2 blockade alone suggesting that P-2 is a common mediator for both, ROS and NO, antimicrobial pathways. In other words without P-2 ROS and NO have little effect on killing of Salmonellae.

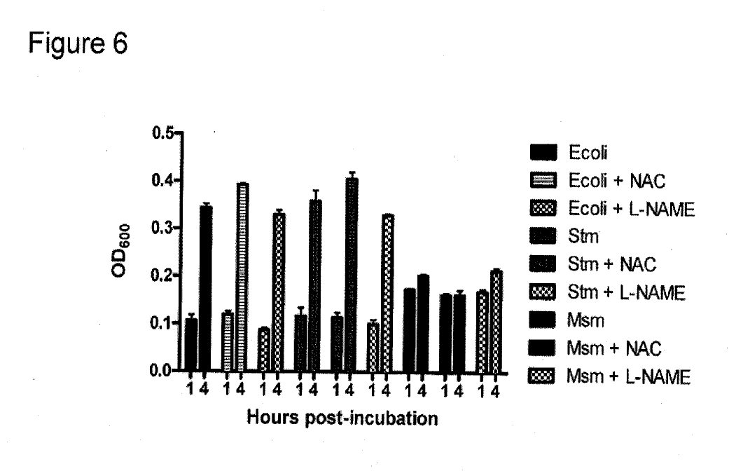

About 30% of M. smegmatis survive 5 h post infection in the absence of inhibitors indicating relative resistance to killing by PEM (FIG. 1B). Blockade of ROS or NO increases their survival to ˜60% providing evidence of a 30% contribution to killing. Knockdown of P-2 on the other hand inhibits almost all bactericidal activity for M. smegmatis suggesting again that P-2 mediates also the toxicity of ROS and NO (FIG. 1B). Non pathogenic E. coli K12 is most sensitive to killing by PEM in the absence of inhibitors (100% killing, FIG. 1C). ROS and NO inhibition or P-2 knock down allow ˜30, ˜40 and ˜60% survival, respectively, indicating that non-pathogenic E. coli is more sensitive to ROS and NO in the absence of P-2 than pathogenic bacteria that may be armed with sturdier cell walls in addition to ROS and NO resistance mechanisms such as production of catalases and antioxidants. The inhibitors, in the absence of PEM, had no effect on bacterial growth (FIG. 6). The data evidence that outer cell wall damage of bacteria by P-2 facilitates the access for ROS and NO to cause irreversible damage to underlying bacterial structures resulting in bacterial death during the first 5 hours of infection. This situation is analogous to Perforin-1 which is required for granzyme-mediated cell death and to the MAC of complement providing access for lysozyme to the bacterial proteoglycan layer resulting in structural collapse.

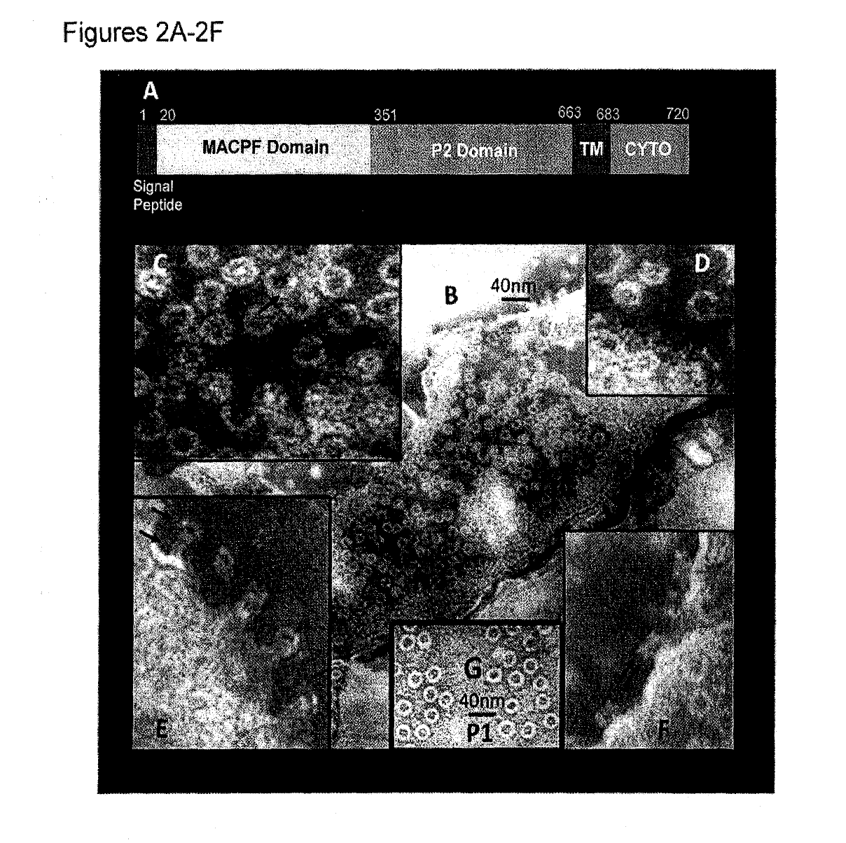

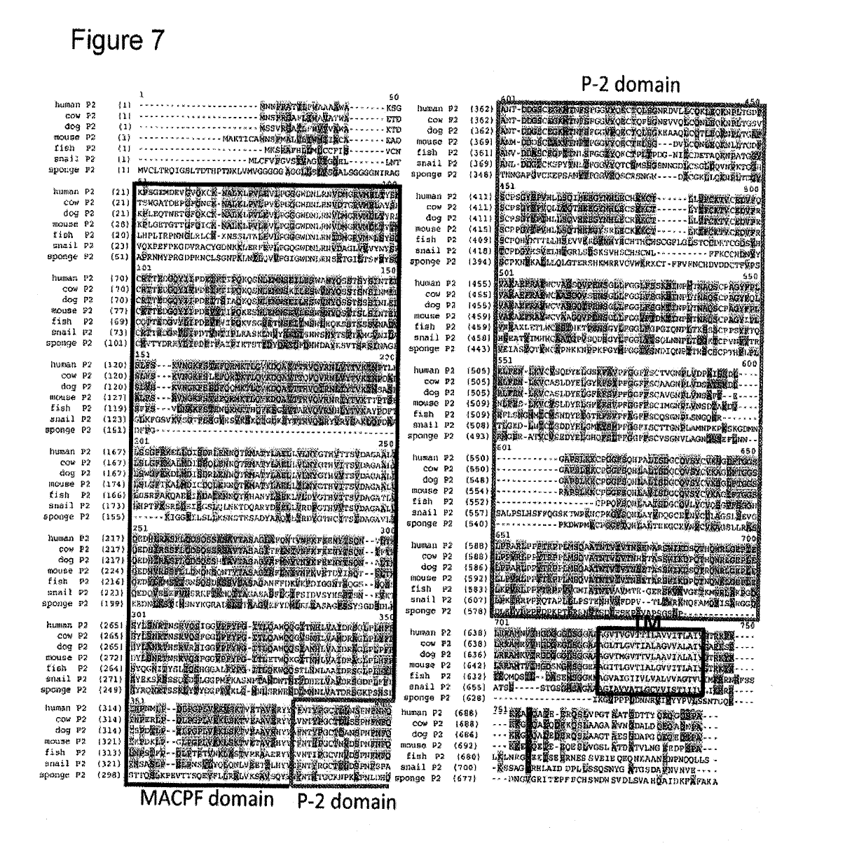

P-2 is highly conserved from sponge to man (FIG. 7), evidencing fundamental functional importance. C-terminal to the MACPF domain is a novel conserved domain, designated here P-2-domain (FIG. 2 A), that is conserved in all Perforin-2 orthologues but does not exhibit homology to any other protein domain. Unlike the other pore formers of the immune system which are soluble, P-2 is a type 1 membrane protein containing a typical transmembrane sequence and a cytoplasmic domain. The effector-MACPF domain points toward the lumen of the ER or budding transport vesicles. The short cytoplasmic domain extends into the cell cytoplasm and displays classical regulatory elements that are currently under study to define the mechanism of P-2 polymerization.

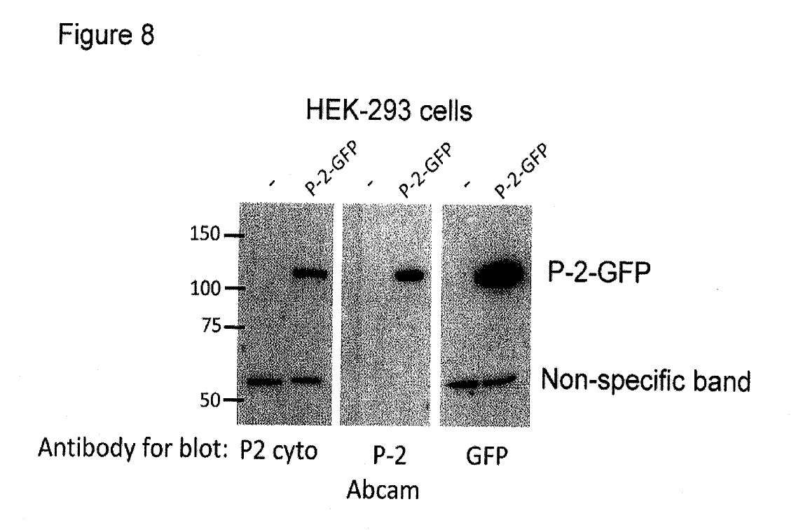

To determine whether P-2 generated membrane associated pores, the complete open reading frame was assembled, fused green fluorescent protein (GFP) to the C-terminus at the cytoplasmic domain, and transfected it into HEK-293 cells. P-2-GFP was detected by immunoblot using a commercial polyclonal anti-peptide antiserum to P-2, the in house polyclonal antiserum raised against the cytoplasmic domain of P-2, or anti-GFP antibodies (FIG. 8). P-2-GFP-fluorescent membranes were obtained by cell lysis and differential centrifugation and treated with trypsin, which removes membrane proteins but not Perforin-1 and MAC pores. Trypsin cleaves the cytoplasmic domain of P-2 but not P-2 pores which remain membrane associated as shown in FIG. 2 B by negative staining electron microscopy at approximately 200,000-fold magnification. This picture represents the first physical evidence and image indicating that P-2 is indeed a pore-forming protein with cytolytic activity. Control membranes from untransfected 293 cells, trypsinized in the same way, were devoid of pore complexes (not shown). Hollow cylindrical poly-P-2 complexes have a mean inner diameter of 9.2 nm compared to poly-Perforin-1 which has a diameter of 16 nm (FIG. 2G) and poly-C9 of 10 nm. The fine structure suggests a polymeric composition of 12-14 protomers. Incompletely assembled pores attest to the polymerization process (FIG. 2C, arrows). In side views, the complex projects outward either 12 or 25 nm (FIGS. 2E and 2F; parallel arrows).

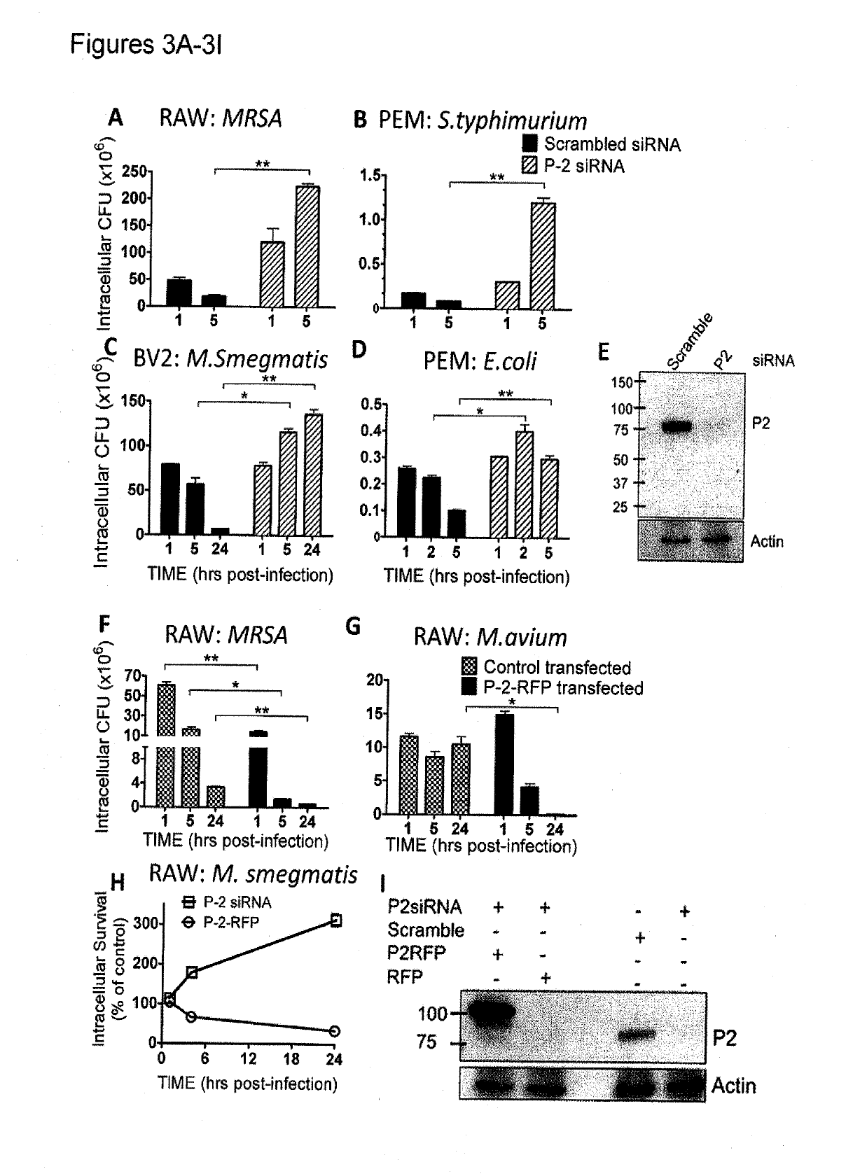

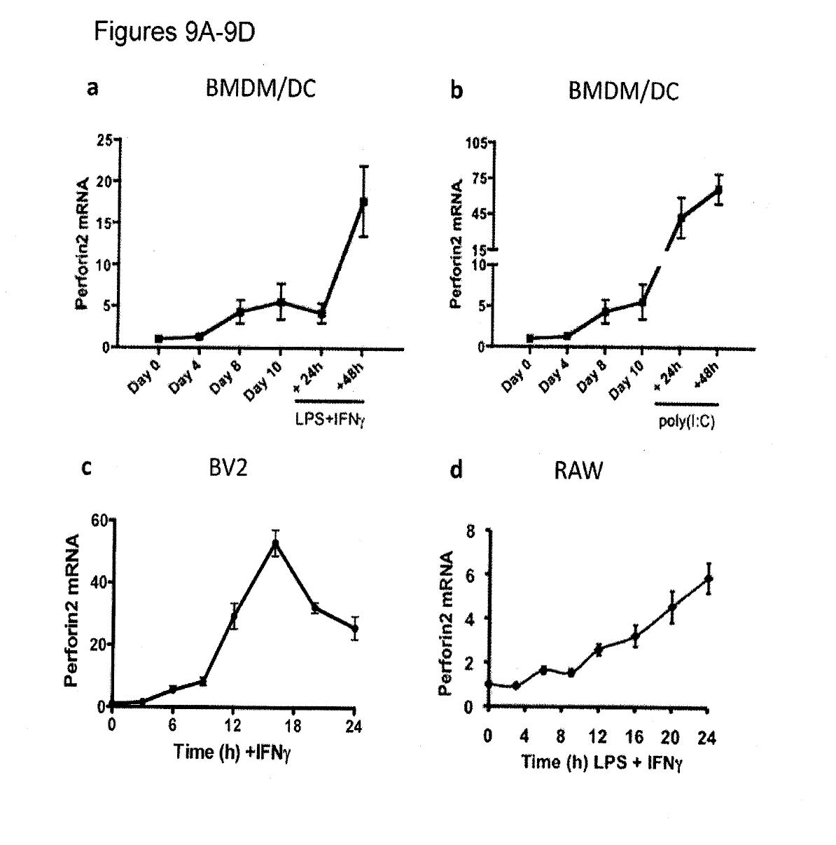

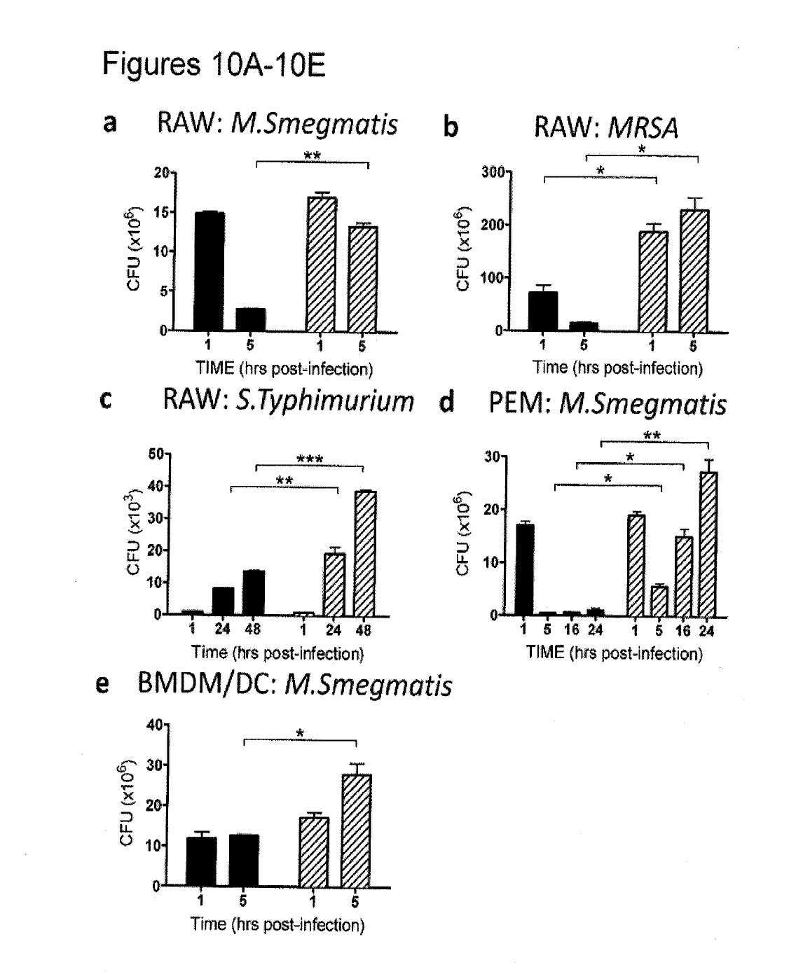

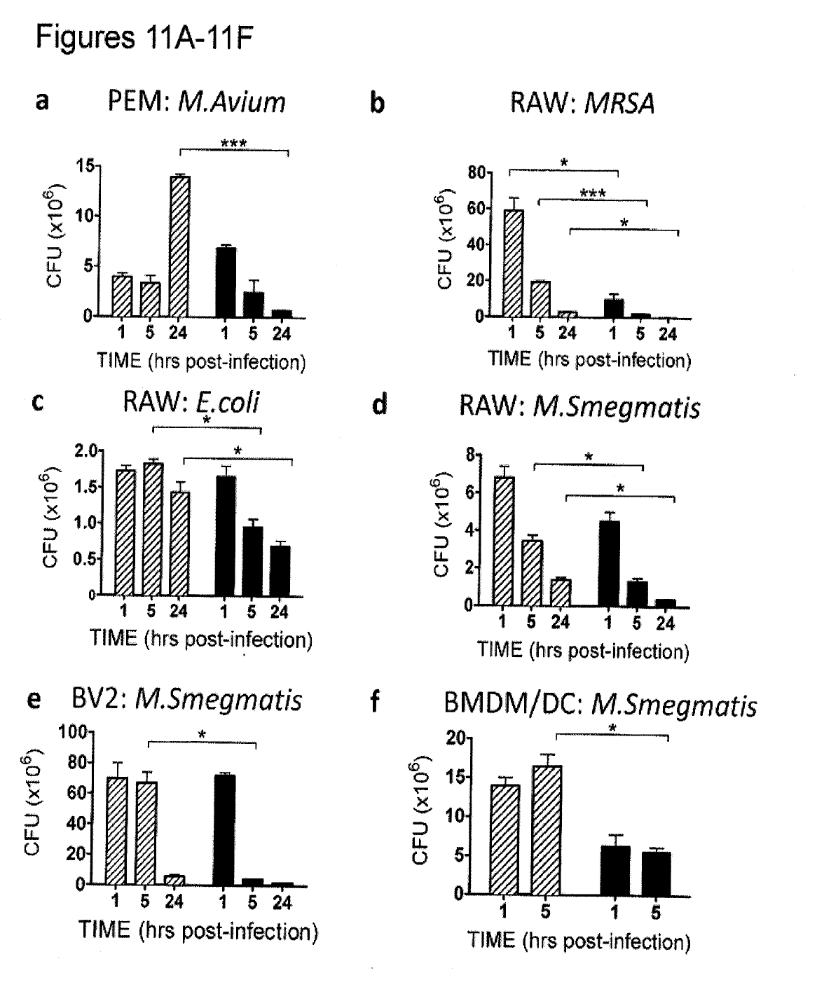

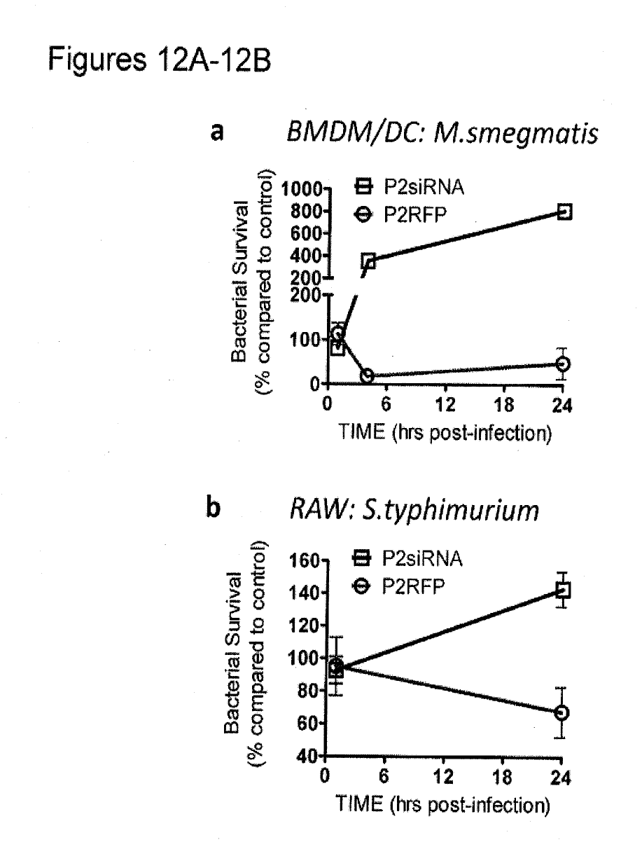

In addition to PEM, bone marrow-derived macrophage/dendritic cells (BMDM/BMDC) and macrophage (RAW264.7) and microglia (BV2) cell lines have potent intracellular killing mechanisms for phagocytosed MRSA, Mycobacteria, S. typhimurium, and E. coli as determined by the gentamycin protection assay (FIGS. 3A-3I). Macrophages, BMDM/DC and microglia express P-2 constitutively and upregulate P-2 mRNA and protein expression upon lipopolysaccharide (LPS) and interferon-gamma (IFN-γ) treatment (FIGS. 9A-9D). P-2 was knocked down in RAW, BV2, BMDM/DC or PEM by transfection with P-2-specific siRNAs and compared intracellular bactericidal activity to cells transfected with scrambled siRNA. Knockdown efficiency of P-2 mRNA was 80 to 95% (FIGS. 5A, 5B) and of protein >90% (FIG. 3E). P-2 knock down strongly inhibited intracellular killing of bacteria in RAW, BV2, BMDM/BMDC and PEM cells (FIGS. 3A-3D and FIGS. 10A-10E). P-2 knockdown inhibited intracellular killing of highly pathogenic M. avium, MRSA and S. typhimurium as well as nonpathogenic E. coli and M. smegmatis (FIG. 3A-3D). Cell viability was not differentially affected in the knockdown and control cells following transfection or during the infection period (not shown). Overexpression of P-2-RFP by transfection of RAW increased intracellular killing of M. avium consistent with intracellular bactericidal functions of P-2 (FIGS. 3F, 3G and FIG. 11A-11F). Cell viability was equivalent in the P-2-RFP expressing and control cells following transfection and during the infection period (not shown). To exclude potential unintended effects of P-2 knockdown on other cellular components that may be responsible for diminished intracellular killing activity, endogenous P-2 was knocked down with P-2-siRNA complementary to the P-2 3′UTR and reconstituted P-2 activity by transfection with P-2-RFP cDNA lacking the P-2 3′UTR (FIG. 3 H and FIGS. 12A, 12B). Knockdown of endogenous P-2 and complementation with P-2-RFP, verified in Western blots (FIG. 3I), fully restored intracellular bactericidal activity, indicating that P-2 is responsible for intracellular bacterial destruction.

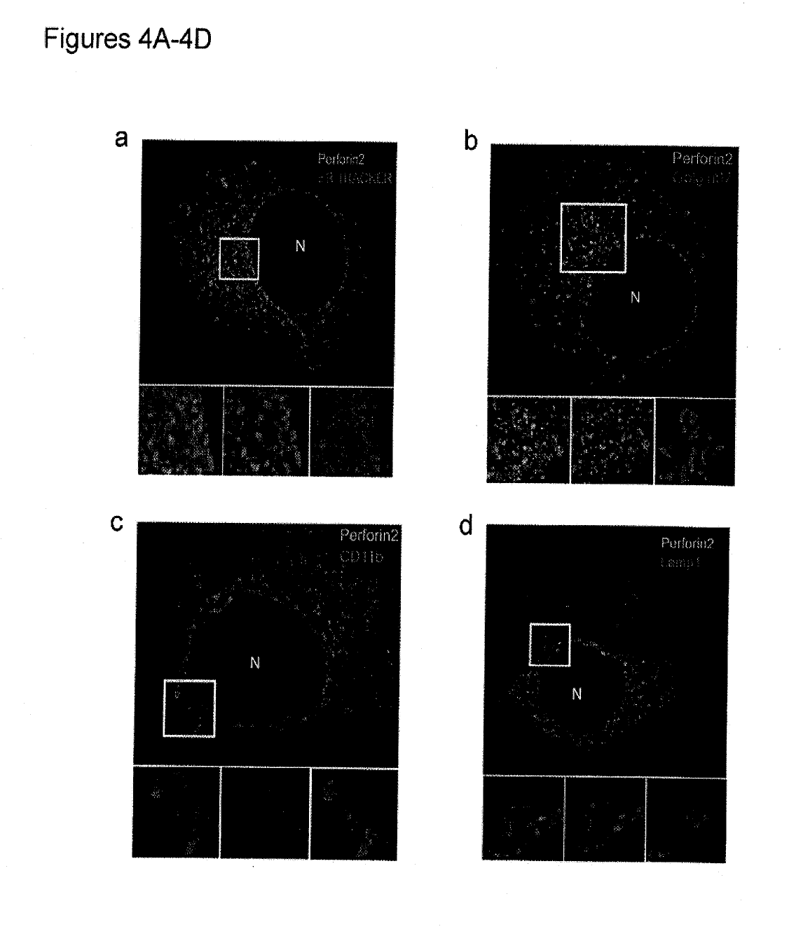

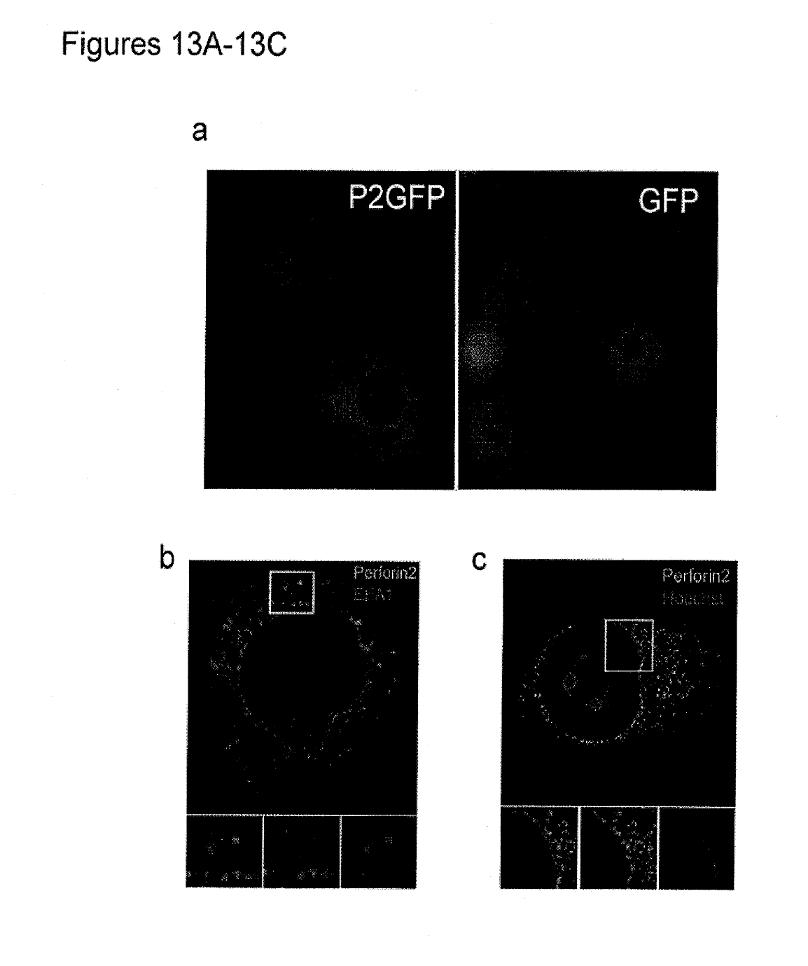

Determination of the intracellular localization of P-2 required transfection of macrophages with P-2-GFP. Commercially available polyclonal anti-peptide antibodies to P-2 do not recognize native P-2 (data not shown); generation of monoclonal antibodies to native P-2 has not been successful so far, probably owing to the high degree of conservation (FIGS. 5A, 5B). To avoid artifacts of P-2-GFP overexpression RAW cells were analyzed after transient transfection at early times when expression of P2-GFP was still low. Unlike GFP alone, P-2-GFP localized primarily to the ER, the Golgi and trans-Golgi network membranes and was excluded from the plasma membrane, lysosomes and early endosomes (FIGS. 4A-4D and FIGS. 13A-13C). The reported fusion of ER membranes with phagosomal membranes allows P-2 access to the phagosome membrane, where it has been detected by mass spectrometry in purified phagosomes containing latex particles of the J774 murine cell line. P-2 therefore is present at the location required for intraphagosomal killing of intracellular bacteria.

The studies herein, establish that macrophages contain a novel pore-forming protein, Perforin-2, which is membrane-associated and has potent intracellular bactericidal functions against a wide range of pathogenic bacterial species. The bactericidal activities of ROS and NO are strongly enhanced by the presence of P-2, the cell wall damaging activity of which may provide access for small molecules including lysozyme to attack the peptidoglycan layer or the inner membrane and DNA of bacteria. The molecular mechanism of the activation of P-2-polymerization is not known but is under active investigation, focusing on the function of the cytoplasmic domain of P-2. Given the long evolutionary antagonism of P-2 and intracellular bacteria it will be of great interest to study evasive strategies of intracellular pathogens for avoiding P-2-attack.

P-2 appears to be the original pore-forming protein of innate immune defense present already in primitive sponges and other invertebrate marine organisms. P-2 is inducible in virtually all body cells and protects them from intracellular bacterial growth. Ancient P-2 continues to be an important antibacterial component of innate immune defense.

### Example 2

### Modulation of P2

It was hypothesized that src kinase would be responsible for P2 activation, and autophagy to be involved in bacterial killing. In addition 7 proteins were identified, with the yeast two hybrid system, that interact with the cytoplasmic domain of P2 and therefore is evidence that they are implicated in P2 activation. These proteins are listed in Table 1.

P2 has a transmembrane domain that is involved in P2 activation for killing. P2 has a highly conserved Y and S and a conserved RKYKKK (SEQ ID NO: 4) domain (GTRKYKKKEYQEIEE; SEQ ID NO: 6).

Knock down of src, Ubiquitin conjugating enzyme E2M (Ubc12), GAPDH, P21 RAS/gap 1m (RASA2), Galectin 3, and UCHL 1 interfered with killing of bacteria by microglia and fibroblasts. These molecules therefore are responsible for activating P2 dependent killing of bacteria. ATG14 knock down also interferes with P2 mediated killing providing a link to P21 RAS and autophagy. By modulating (blocking or enhancing) these P2 activator proteins with small drugs or biologics would result in the increase or decrease P2 activity. Since P2 is expressed in many if not all tissues it clearly is of extreme importance for anti-microbial control. At the same time dysregulation may lead to auto aggressive and autoimmune disease (up regulated activity) or to immune deficiency (down regulation). Pathogenic bacteria are likely to interfere with P2 activation via blocking the activation cascade. Counteracting such interference could provide to treat and cure patients with infections with drug resistant bacteria.

In summary the molecular mechanisms of P2 activation provides many druggable targets that could be useful foe a broad spectrum of diseases.

TABLE 1

| | Clone | Number of clones | Homology |
|---|---|---|---|
| 1 | Ubiquitin-conjugating enzyme E2M | 18 | 100% |
| | (Ubc12) | | |
| 2 | GAPDH | 17 | 100% |
| 3 | P21 RAS activator2/gap1m (sfpil) | 13 | 100% |
| | (RASA2) | | |
| 4 | Galactose binding lectin (Galectin-3) | 6 | 100% |
| 5 | Ubiquitin C (UCHL1) | 1 | 100% |
| 6 | Chromosome #6 | 1 | 100% |
| 7 | Mus musculus proteasome | 1 | 100% |

### Example 3

### Somatic Cells Mediate Bactericidal Functions Via Pore Forming Protein Perforin-2

Macrophages, dendritic cells, and microglia constitutively express a pore-forming protein, designated Perforin-2 (P-2), to kill pathogenic intracellular bacteria. P-2 kills bacteria by membrane damage which also enhances the bactericidal effects of reactive oxygen species (ROS) and nitric oxide (NO), evidencing that physical damage provides access to sensitive layers of bacteria. Epithelial cells, fibroblasts, and other non-hematopoietic-derived cells are invaded by bacteria and clear intracellular bacterial infections with the aid of autophagy-related mechanisms and antimicrobial compounds. Prior to these experiments, it was unknown whether P-2 could be expressed and used by non-hematopoietic cells for bacterial clearance. This is the first report to show that primary human keratinocytes express P-2-mRNA constitutively and that all somatic cells analyzed to date can be induced to express P-2 mRNA by type 1 and type 2 interferons. Moreover knockdown of endogenous, P-2 with a P-2-specific siRNA 1 inhibits bactericidal activity, which is restored by complementation with P-2-RFP, but not by RFP.

Materials and Methods

Human Cells:

The following cells were used. Umbilical vein endothelial cells, MIA PaCa-2 pancreatic cancer (ATCC CRL-1420), UM-UC-3 bladder cancer (ATCC CRL-1749), UM-UC-9 bladder cancer23, HeLa (ATCC CCL-2), HEK293T (ATCC CRL-1573), and primary keratinocytes. HUVECs were grown in Lonza EGM-2 bullet kit; primary human keratinocytes were grown as previously described (Wiens, M. et al. J Biol Chem 280, 27949-27959, doi:10.1074/jbc.M504049200 (2005)). All other cells were grown following ATCC recommendations. All cells were cultured at 37° C. in a humidified atmosphere containing 5% CO₂.

Mouse Cells:

CT26 colon carcinoma (ATCC CRL-2638), CMT-93 rectal carcinoma (ATCC CCL-223), B16-F10 melanoma (ATCC CRL-6475), Neuro-2a neuroblastoma CATH.a neuroblastoma. Ovarian caricinoma's MOVCAR 5009 and MOVCAR 5047 were purchased from Fox Chase cancer center. NIH/3T3 fibroblast (ATCC CRL-1658), C2C12 myoblast (ATCC CRL-1772), primary meningeal fibroblasts, and primary astrocytes were isolated as previously described (Sabichi, A. et al. The Journal of Urology 175, 1133-1137, doi:10.1016/S0022-5347(05)00323-X (2006); Tomic-Canic, M. et al. Wound repair and regeneration: official publication of the Wound Healing Society [and] the European Tissue Repair Society 15, 71-79, doi:10.1111/j.1524-475X.2006.00187.x (2007)). All cell lines were grown in accordance with ATCC guidelines, and culture at 37° C. in a humidified atmosphere containing 5% CO₂.

Chemicals:

MG-132, Chicken egg white Lysozyme, and Lipopolysaccharide (LPS) were purchased from Sigma. Recombinant murine IL-1α, IL-1β, TNFα, IFN-γ, IFN-α, IFN-β recombinant human IFN-γ, IFN-β were purchased from preprotech. Recombant human IFN-α was purchased from R&D systems. Murine IL-1β was supplemented at 10 U/mL where indicated. Murine IL-1β was supplemented at 1 ng/ml. Murine TNFα was supplemented at 20 ng/ml. Murine IFN-α, IFN-β and IFN-γ was supplemented with 100 U/ml. Human IFN-α was supplemented where indicated in at a concentration of 150 U/ml where indicated. Human IFN-β and IFN-γ was supplemented at a concentration of 100 U/ml. LPS was added at a concentration of 1 ng/ml.

Plasmid Constructs:

The complete coding region of murine Mpeg-1 cDNA was constructed from several EST clones and inserted into the pEGFP-N3 plasmid (Clontech). Monomeric RFP was cloned in place of GFP for use in infection experiments.

Negative Staining Transmission Electron Microscopy:

mEF were stimulated for 14 hours with IFN-γ (100 U/mL) and infected with the indicated bacterial strains at a multiplicity of infection of 30 for 5 hours. Prokaryote membranes were harvested through lysing mEFs with 1% Igepal in ddH2O. The lysate was centrifuged at 200 g for 10 minutes to pellet intact bacteria. The resulting pellet was resuspended in in minimal ddH2O washed and negatively stained with 3% Uranyl Formate (UF) for 30 seconds. Images were taken at 52,000-fold initial magnification on a Phillips CM10 transmission electron microscope.

Antibodies:

Rabbit anti-Mpeg1 polyclonal antibody was obtained from Abcam and used for western blot analysis.

qRT-PCR:

RNA was extracted from cells following RNeasy (Qiagen) instructions. One μg of RNA was converted to cDNA using QuantiTect Reverse Transcription kit (Qiagen) following supplier's protocol. qRT-PCR was performed using TAQMAN® Gene Expression Assays (Applied Biosystems) for murine Mpeg1 and GAPDH, with the later serving as a housekeeping control gene. For human tissues, human Mpeg1 and GapDH probes were utilized. All assays were performed on the Applied Biosystems 7300 PCR platform.

RNA Interference:

For murine cells, three mpeg1-specific chemically synthesized 19-nucleotide siRNA duplexes were obtained from Sigma. Two siRNAs were complementary to the 3′ UTR of P-2 and the third to the coding region. The sequences were as follows: CCACCUCACUUUCUAUCAA (SEQ ID NO:1), GAGUAUUCUAGGAAACUUU (SEQ ID NO:2), and CAAUCAAGCUCUUGUGCAC (SEQ ID NO:3). A scramble siRNA was also generated to serve as a control to the reaction. For human cells, three human mpeg1-specific silencer select siRNA were purchased from Ambion (Invitrogen) Silencer Select #s61053, s47810, s61054. Silencer select negative control #2 from Ambion (Invitrogen) was also used.

Transfections:

Transient transfections were carried out utilizing the Amaxa Nucleofector System (Lonza) according to the manufacturer's optimized protocol for each cell line.

Gentamycin Protection Assay:

S. typhimurium strain LT2Z), Mycobacterium smegmatis, methicillin-resistant Staphylococcus aureus, and E. coli strain K12 were grown from glycerol stocks at 37° C. with shaking for 24 hours in Luria broth (S. typhimurium, S. aureus, and E. coli) or Middlebrook 7H9 broth (M. smegmatis). For S. typhimurium, S. aureus, and E. coli, these cultures were then diluted 1:33 in LB and grown for another 3 hours to reach log phase prior to infection. Eukaryotic cells were transfected following Lonza's optimized protocol for the respective cells, and plated into 12 well plates post transfection. The cells were then stimulated for 14 hours with IFN-γ (100 U/ml). Cells were infected at a multiplicity of infection (MOI) between 10 and 60 for 30 minutes (S. typhimurium) or 1 hour (S. aureus, E. coli, and M. smegmatis) in 37° C., 5% CO₂ incubator. After infection, cells were washed twice with ice-cold PBS and fresh media containing 50-μg/ml gentamycin was added. After 2 hours, the media was changed to decrease the concentration of gentamycin to 5 μg/ml. At indicated time points, cells were washed with PBS, lysed using 1% Igepal in ddH2O, diluted and plated in technical triplicate on LB agar plates (S. typhimurium, S. aureus, E. coli) or Middlebrook 7H11 plates (M. smegmatis) and CFU determined after sufficient colony growth.

Lysozyme Killing Activity:

Follow above for Gentamycin protection assay, after lysis, divide the lysate into 6 equal fractions, treating half to achieve final concentration 40-μg/ml lysozyme and the remainder with equal volume buffer. All fractions were incubated on ice for 30 minutes prior to plating in technical duplicates for CFU analysis.

Statistical Analysis:

The data was first analyzed according to the Kolmogorov-Smirnov test (K-S test) to determine if a Gaussian distribution is present. Using the resulting statistic, the data was analyzed according to the number of independent variables in each experiment. If comparing between two groups, and the data fits a Gaussian distribution according to the K-S test, an independent measures t test was used; however, if a Gaussian distribution is not present a Mann-Whitney test was carried out in order to assess statistical significance. For analysis of greater than two groups, one-way, independent measures ANOVA applying a Bonferroni post hoc test, if a Gaussian distribution is present. If a Gaussian distribution is not present, the Kruskal-Wallis test is used utilizing a Bonferroni post hoc test.

Results and Discussion

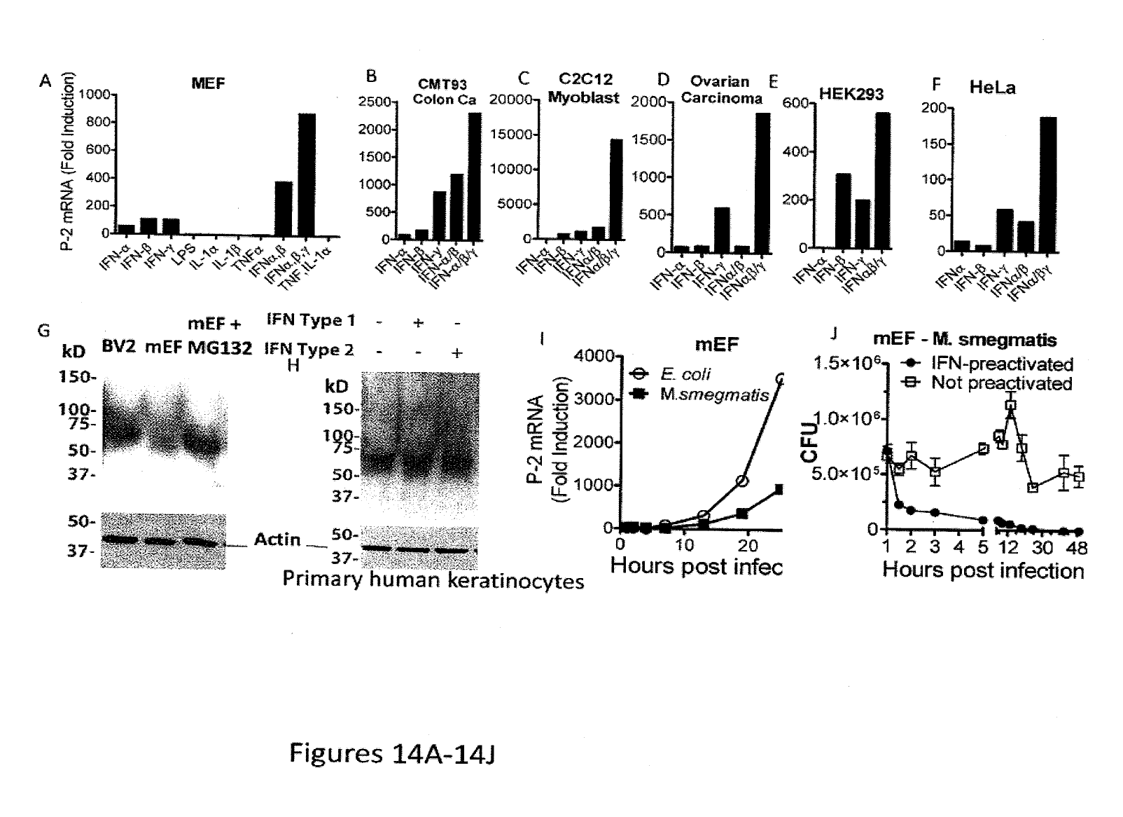

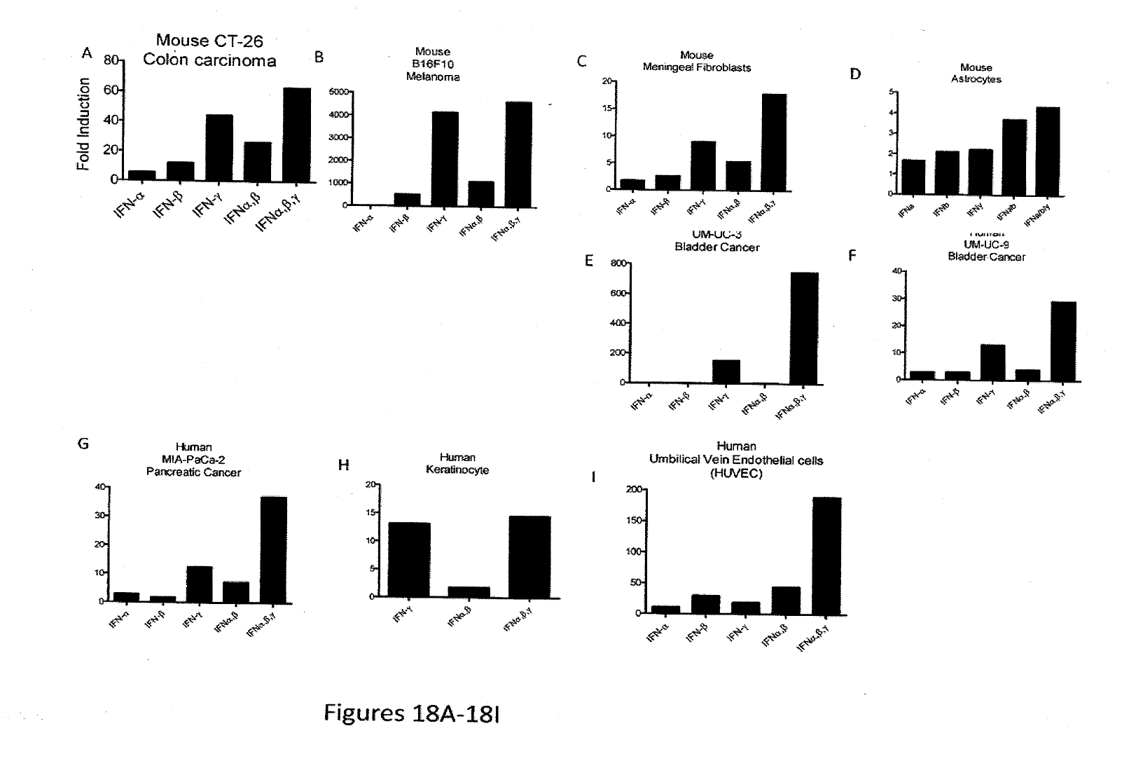

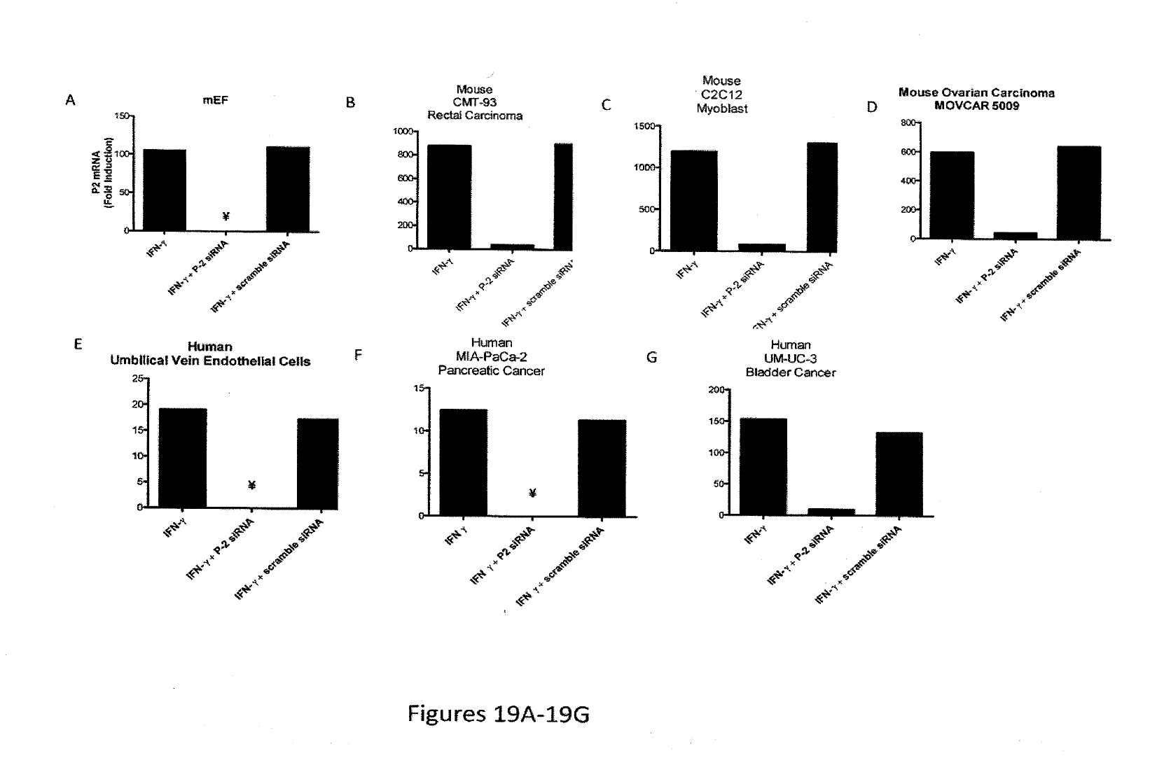

All human and mouse primary cells and cell lines analyzed to date rapidly express P-2 mRNA upon interferon induction (FIGS. 14A-14J and Table 2 and FIGS. 18A-18I). Unstimulated primary murine embryonic fibroblasts (mEF) do not express detectable levels of P-2 mRNA by TAQMAN™ PCR after 39 cycles (FIG. 14 A). Interferon (IFNα, β or γ added singly each up-regulates P-2 mRNA; added together they induce high levels of P-2 mRNA. LPS, IL-1α, and TNFα in contrast do not induce P-2 mRNA in mEF. Although there are individual differences, this pattern of P-2 induction was found in all primary cells and established cell lines tested from mouse and man (FIGS. 14A-14F, complete listing in FIGS. 18A-18I and Table 2). Despite potent upregulation of P-2 mRNA by IFNs, P-2 protein is not detected by Western blot analysis of interferon-activated mEF unless the proteasome inhibitor MG132 is added (FIG. 14G), evidencing that rapid P-2 protein turnover is occurring through proteasomal degradation. The exception to these findings occurs with primary human keratinocytes that constitutively express P-2 mRNA and protein (FIG. 14H).

LPS is unable to induce P-2 mRNA in mEF; however, coincubation of mEF with E. coli K12 or Mycobacterium (M.) smegmatis results in a strong increase in the expression of P-2 mRNA within 24 h with significant upregulation of P-2 mRNA levels detected already at 10 h (FIG. 14I). Preinduction of P-2 with type 2 IFN enables mEF to rapidly kill intracellular M. smegmatis within 1-5 hours in the gentamycin protection assay (FIG. 14J); gentamycin is membrane impermeable during this period in intact cells. In contrast, when uninduced mEF are incubated with M. smegmatis, intracellular killing of mycobacteria is delayed by about 16 h and is less efficient (FIG. 14J). The association of P-2 mRNA levels with intracellular bactericidal activity suggests a causal relationship.

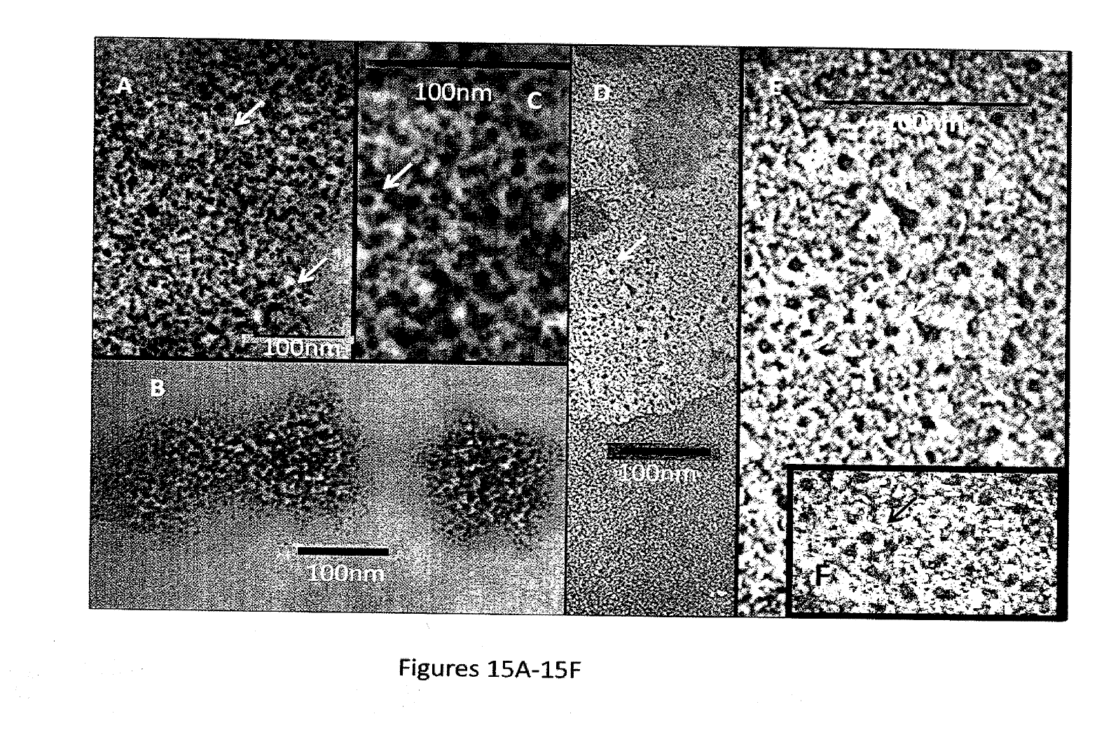

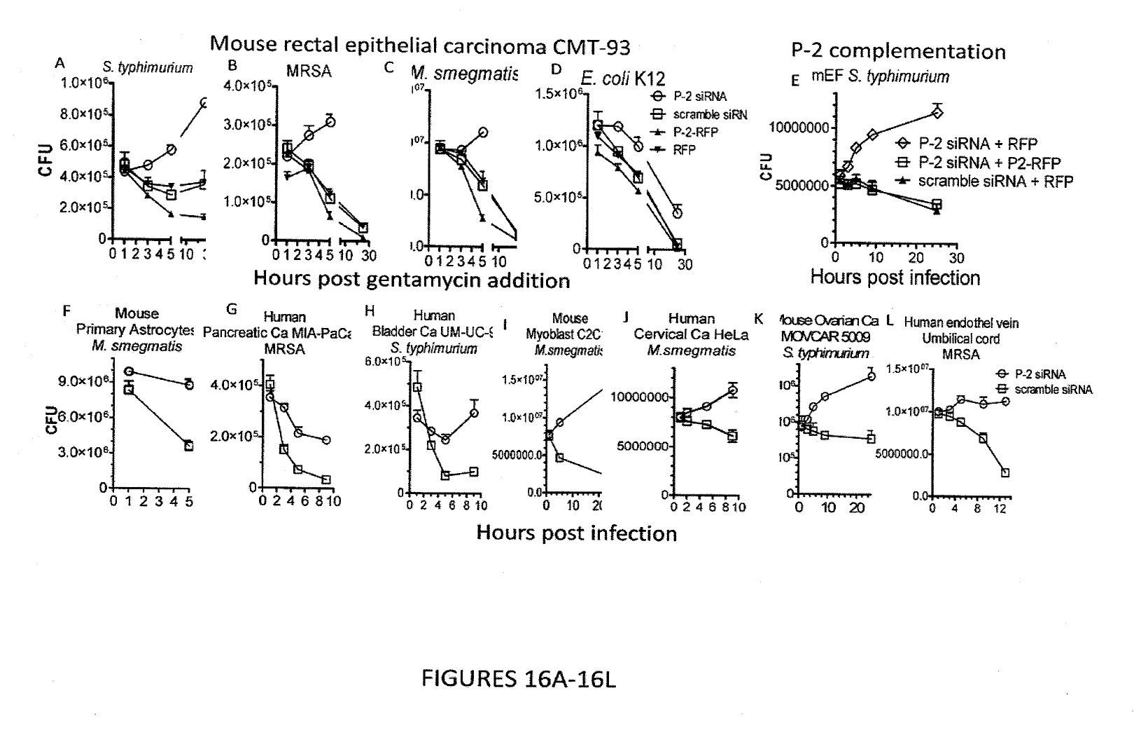

P-2 mediated killing of bacteria predicts (1) electron microscopic lesions on cell walls of bacteria killed by mEF and (2) inhibition of intracellular killing when P-2 mRNA is knocked down with P2-siRNA. Intracellular M. smegmatis or Methicillin-resistant Staphylococcus aureus (MRSA) were isolated from type 2-IFN-induced mEF by detergent lysis of host cells 5 h after infection. At this time most of the intracellular mycobacteria are dead as indicated by lack of colony formation (FIG. 14J). The bacteria are separated from the cell lysates by centrifugation. Because membrane-bound P-2-polymers are resistant to trypsin cleavage, similar to poly Perforin-1 and poly C9, bacterial pellets were treated with trypsin and lysozyme and then inspected by negative staining electron microscopy at 250,000× magnification. The images of mycobacterial and staphylococcal membranes show lesions conforming to the expected morphology of poly P-2 (FIG. 15A, 15B, 15D, 15E) which are similar to the positive control of P-2 stable overexpression on HEK293 membranes (FIG. 15C) and Membrane Attack Complex (MAC) of complement lesions on E. coli (FIG. 15F). Clusters of circular or irregularly fused, negative stain-filled lesions of 9-10 nm mean internal diameter (arrows, FIG. 15A) are seen on the otherwise smooth background of MRSA cell walls, shown at higher magnification in FIG. 15B. Similar membrane lesions are seen on M. smegmatis membranes (arrows FIGS. 15D and 15E) shown at two magnifications. Clusters of P-2 lesions are seen in patches on bacterial membranes suggesting that bacteria make local contact with P-2 bearing membranes resulting in P-2 polymerization and poly-P2 insertion into the outer bacterial cell wall creating the lesions seen in the electron-microscope. Rectal epithelial carcinoma cells CMT93 kill M. smegmatis, clinical isolates of MRSA, S. typhimurium, and E. coli K12 in the gentamycin protection assay (FIGS. 16A-16D). Killing is enhanced upon transfection of CMT-93 with RFP-tagged P-2-RFP but not by RFP alone. In contrast, P-2 siRNA, but not scrambled siRNA transfection eliminates bactericidal activity, causing all tested bacteria except E. coli to replicate intracellularly. MRSA and M. smegmatis kill host cells owing to this replication after several hours and are released into the medium containing gentamycin where they are killed by the antibiotic. Assays were limited in most cases to the first 5 hours when most P-2-dependent bacterial killing occurs. Bacterial killing in cells depleted of endogenous P-2 could be restored by transfection with P-2-RFP that is resistant to knockdown due to its lack of the 3′UTR of endogenous P-2 (FIG. 16E).

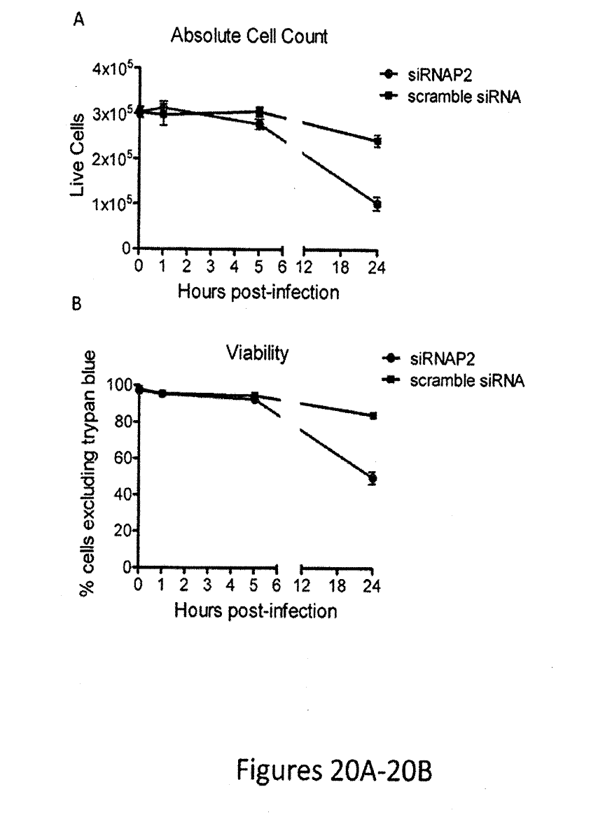

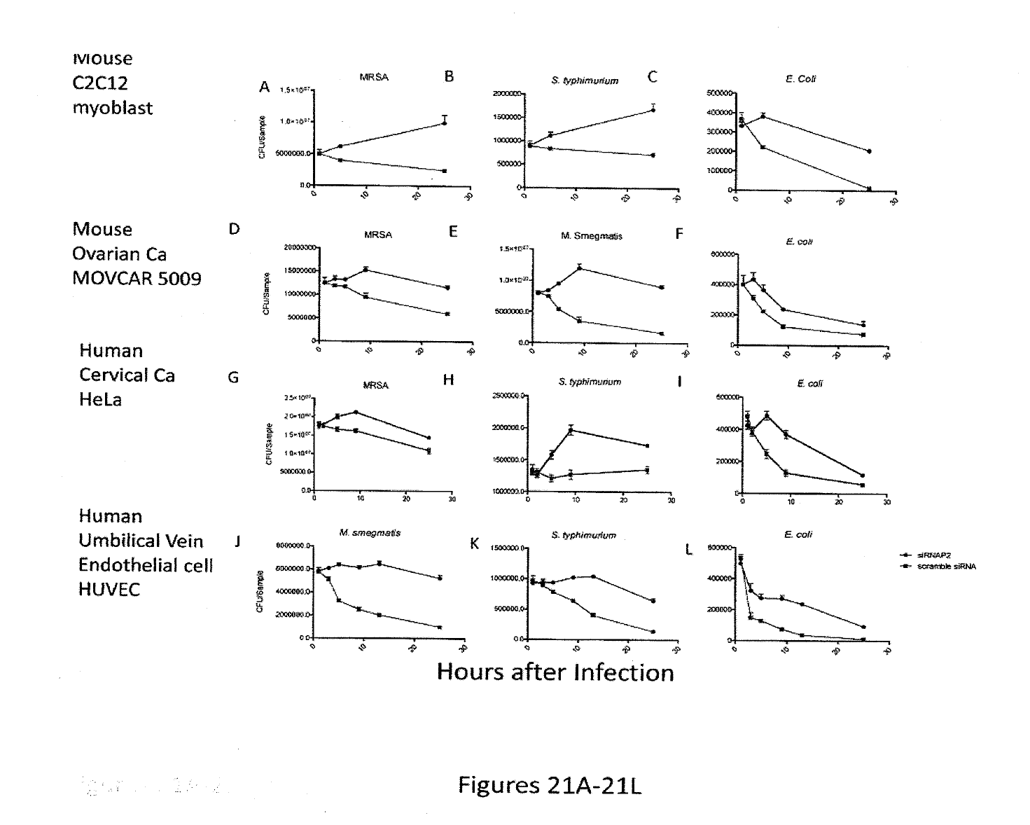

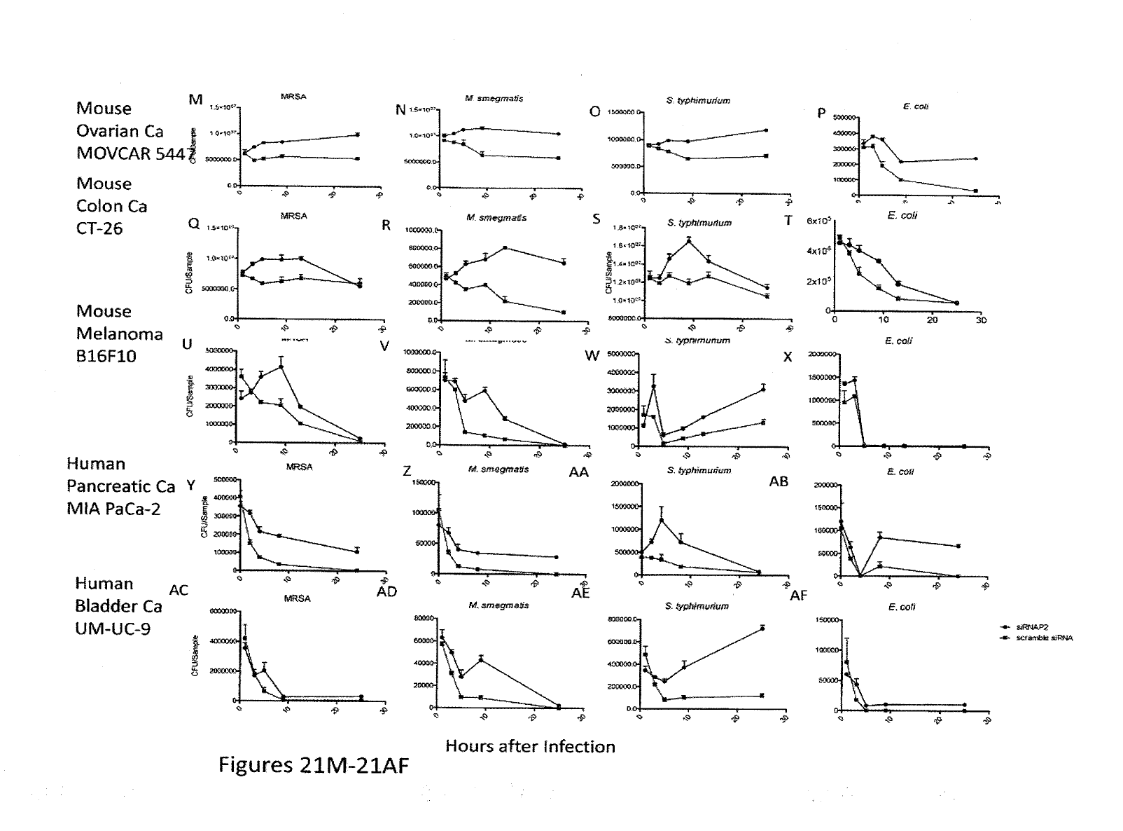

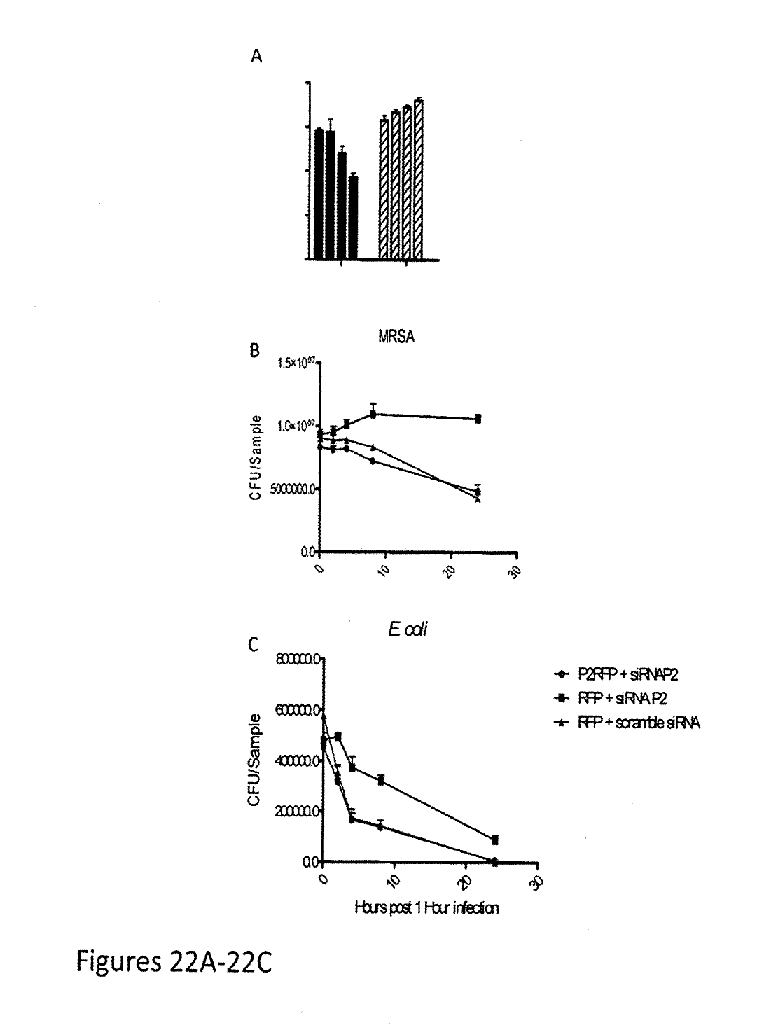

All mouse and human cells expressing constitutive or inducible P-2 mRNA are able to kill all of the four bacterial strains. P-2 siRNA inhibited intracellular killing activity as shown in the examples in FIGS. 16F-16L and in supplementary FIGS. 20A-20B, evidencing that P-2 mediated intracellular killing of bacteria is a critical component of natural immunity preventing intracellular bacterial invasion.

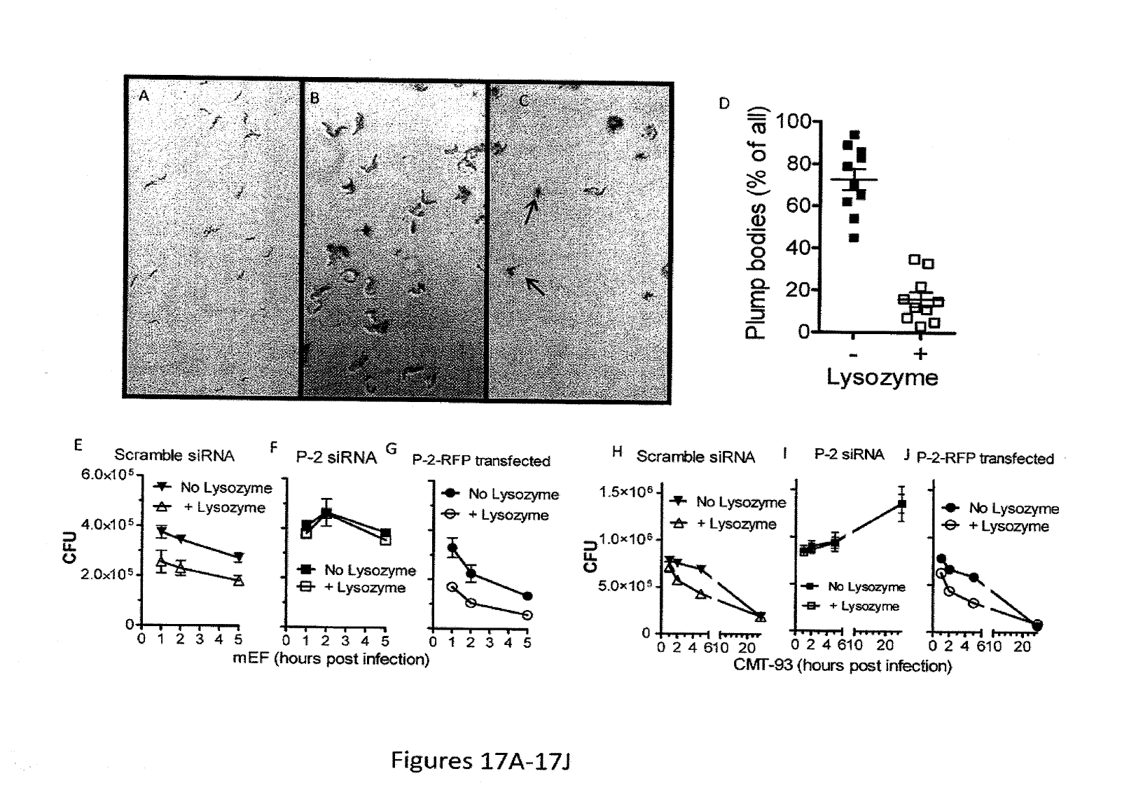

Inspection of plated bacteria with the phase contrast light microscope allows early determination and counting of colonies in the colony-forming assays, saving time especially for Mycobacteria which require 2-3 days to form colonies visible by eye, but detectable by microscopy already 12 h after plating. Inspecting M. smegmatis in this way, it was noted that the majority of bacteria isolated from host cells after 5 h had swollen, plump bodies that did not form colonies, suggesting that they are dead (FIGS. 17A, 17B). Live mycobacteria plated fresh from culture have corkscrew morphology (FIG. 17A) and can form colonies (not shown). In vitro addition of lysozyme for 30 minutes on ice to mycobacteria isolated at 5 h (FIG. 17C) causes the disappearance of most of the plump bodies suggesting that their lysis occurred, but did not affect the corkscrew morphology of the few live bacteria which are beginning to form colonies (arrows FIG. 17C). Poly-P-2 lesions in bacterial cell walls therefore mediate susceptibility to lysis by lysozyme which is quantitated by counting all bacteria and reporting the percentage of plump bodies with and without lysozyme addition (FIG. 17E).

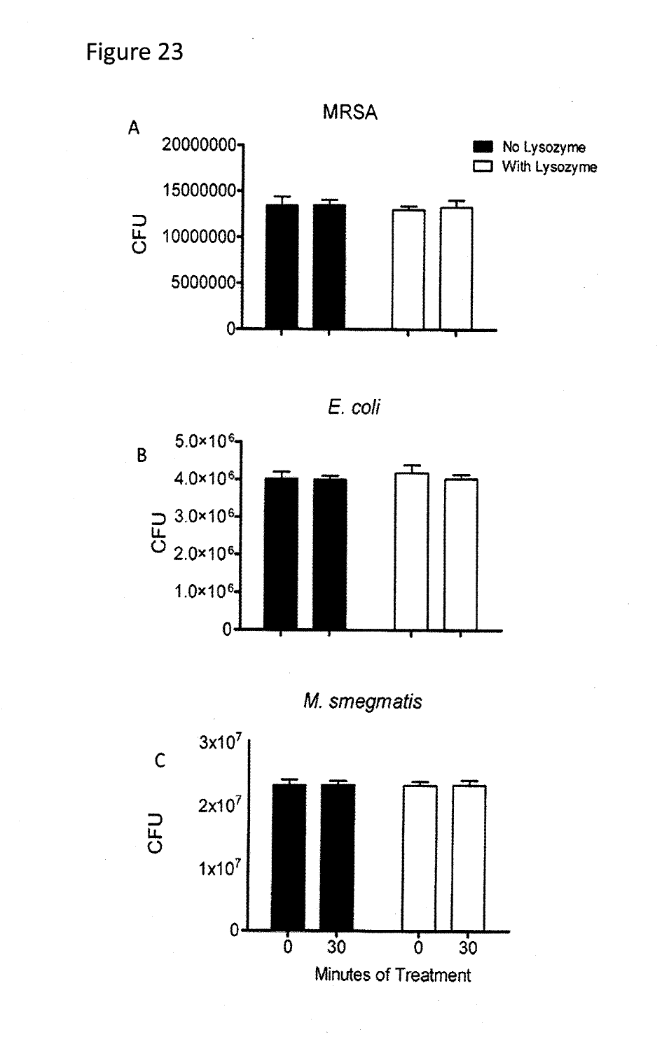

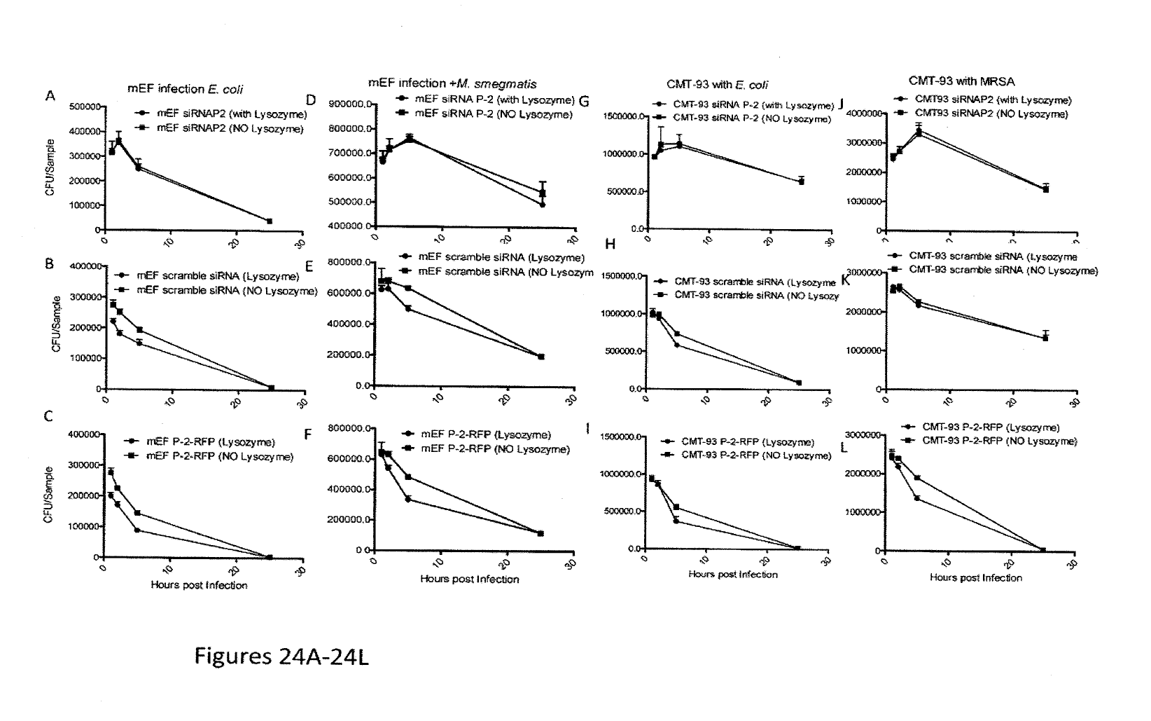

The influence of P-2 damage to the cell wall on the effect of lysozyme was further studied by P-2 knockdown and P-2 overexpression in mEF and in CMT93 cells using M. smegmatis, MRSA, and E. coli all of which are lysozyme-resistant when undamaged (FIGS. 17E-17J, FIGS. 23A-23C). In scramble siRNA controls containing normal P-2 levels, the addition of lysozyme to bacteria obtained at different times after detergent lysis of host cells significantly reduces the number of colonies (FIGS. 17E and 17H) evidencing that some bacteria have damaged cell walls that; however, can be repaired, unless the bacteria are lysed by lysozyme that gains access via cell wall damage through by poly-P-2. The bactericidal effect of lysozyme is not detected when P-2 is knocked down by P-2 siRNA (FIGS. 17F and 17I) indicating absence of poly-P2-mediated cell wall damage, but is more pronounced when P-2-RFP is overexpressed together with endogenous P-2, leading to more P-2-mediated cell wall damage (FIGS. 17G and 17J).

The above data indicate that apparently all of our cells have the ability to become killer cells and eliminate intracellular bacterial invasion with the aid of the pore-forming protein Perforin-2. P-2 acts very early, damaging the bacterial cell wall by insertion into the lipid layer and polymerization, analogous to C9 polymerization during membrane attack by complement and to Perforin-1 polymerization during CTL attack of virus-infected cells and neoplastic cells. All three-pore formers share the MACPF domain which has been shown to trigger polymerization in Perforin-1 and is likely to mediate the same function in Perforin-2. Poly-C9 pores of the MAC provide access for serum lysozyme leading to bacterial lysis and structural collapse; likewise poly-Perforin-1 pores provide access for granzymes that mediate cell death via multiple apoptotic and non-apoptotic pathways. In analogy, poly-Perforin-2 pores provide access for lysozyme, ROS, NO, and probably other anti-microbial compounds to enhance bacterial killing. Physical membrane damage by pore-forming proteins thus is a common mode of immune defense. Perforin-2 kills intracellular bacteria, the MAC kills extracellular bacteria and Perforin-1 kills virus infected cells via physical attack. In all cases, the cell wall/membrane damage caused by the pore formers serves as entry port for additional cytotoxic molecules to finish the task. Of the three pore formers sharing the MAC/PF domain, Perforin-2 appears to be the oldest, being present already in sponges and other invertebrates. It differs from the other two pore formers in being a transmembrane protein that is activated by transmembrane signaling from the cytoplasmic domain. Elucidation of the signaling mechanism is likely to offer drug targets to enhance or diminish P-2 activity and to unveil bacterial evasion mechanisms.

TABLE 2
Summary of all cell lines tested. N.D. is listed when P-2 dependent
killing was not done.

| | P-2 mRNA IFN- | |
|---|---|---|
| Cell type | inducible? | P-2 dependent killing? |
| CT26 colon carcinoma | Inducible | Yes |
| (M.m) | | |
| Primary CNS fibroblast | Inducible | Yes |
| (M.m) | | |
| Primary keratinocytes | Constitutive | N.D. |
| (H.s) | | |
| B16F10 melanoma (M.m) | Inducible | Yes |
| Neuro2.A neuroblastoma | Inducible | N.D. |
| (M.m) | | |
| Ubc9 bladder cancer | Inducible | Yes |
| (H.s) | | |
| MiaPac pancreatic | Inducible | Yes |
| cancer (H.s) | | |
| Cath.A neuroblastoma | Inducible | N.D. |
| (M.m) | | |
| Ubc3 bladder cancer | Inducible | Yes |
| (H.s) | | |
| murine Embryonic | Inducible | Yes |
| Fibroblast (mEF) (M.m.) | | |
| NIH 3T3 (M.m.) | Inducible | Yes |
| C2C12 myoblast (M.m) | Inducible | Yes |
| CMT93 colon carcinoma | Inducible | Yes |
| (M.m). | | |
| Primary astrocytes | Inducible | Yes |
| (M.m) | | |
| HeLa cervical carcinoma | Inducible | Yes |
| (H.s.) | | |
| 293 Embryonal kidney | Inducible | Yes |
| (H.s.) | | |
| Umbilical endothelial | Inducible | Yes |
| cells (H.s.) | | |
| Peritoneal macrophages | Constitutive | Yes |
| (M.m) | | |
| Bone marrow derived DC | Constitutive | Yes |
| (M.m) | | |
| Microglia (M.m.) | Constitutive | Yes |
| Polymorph-nuclear | Constitutive | N.D. |
| neutrophilic ganulocyte | | |
| (H.s.) | | |
| HL60 promyelocyte | Constitutive | Yes |
| PMN (H.s) | | |

### Example 4

### Perforin-2 Protects Against Lethal Bacterial Infection

Reactive oxygen and nitrogen intermediates and permeability increasing proteins are important antibacterial effectors, however additional bactericidal effectors are thought to exist. We now show that the pore-forming protein Perforin-2 is essential for protection against intracellular bacterial replication. Ubiquitous throughout the body, Perforin-2 kills Gram positive, negative and acid fast bacteria. Perforin-2 deficient mice die from oro-gastric Salmonella infections that are cleared in sufficient mice. Perforin-2 is a transmembrane protein pointing its MACPF-killer-domain into the lumen of membrane-vesicles that translocate to the bacterium containing vacuole upon infection. Perforin-2 killed bacteria bear clustered 90 Å pores on their cell wall that may render bacteria more susceptible to reactive oxygen and nitrogen intermediates. Pathogenic bacteria subvert Perforin-2 expression or activation and Perforin-2 levels are suppressed in non-healing, chronically infected skin ulcers in patients. Studying the pathways of Perforin-2 action will provide opportunities for novel approaches against life threatening bacterial infections.

### Perforin-2 Knock Out in Mice Results in Uncontrolled Salmonella Replication and Lethality

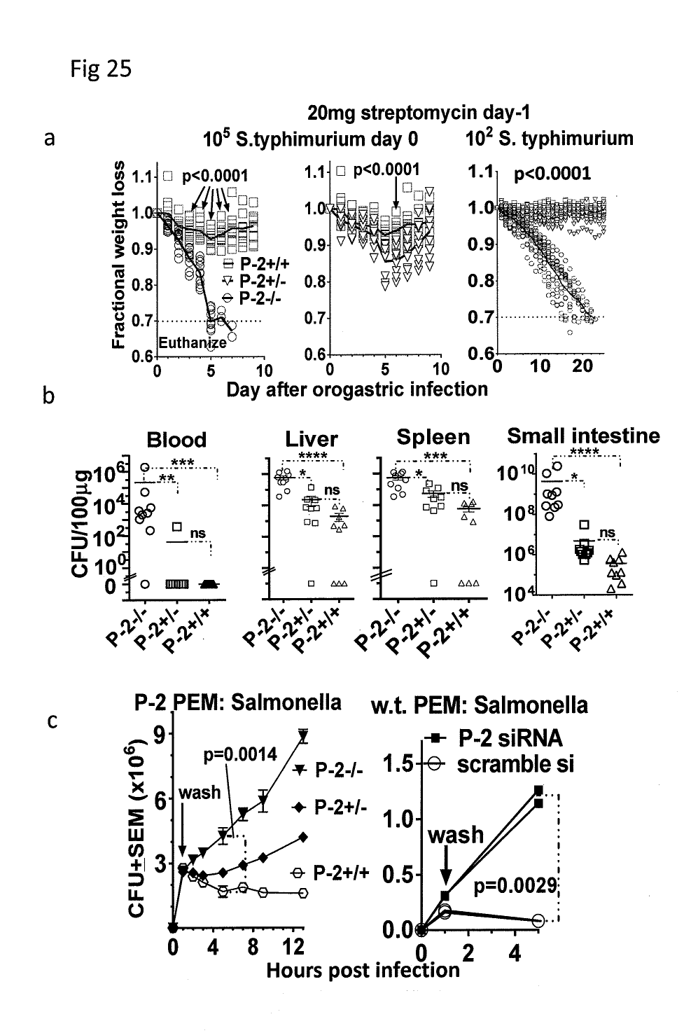

To determine the biological importance of Perforin-2 in vivo we generated Perforin-2 knock out (P-2−/−) mice by homologous recombination. The mice are of mixed C57Bl6 and 129 backgrounds providing for differences in minor MHC antigens and thereby generating limited diversity. P-2−/− mice develop and thrive normally under pathogen free conditions. Homozygous P-2−/−, heterozygous P-2+/− and wild type P-2+/+ littermates were challenged oro-gastrically with Salmonella typhimurium as described. Infection of P2+/+ mice with 10⁵ streptomycin resistant salmonellae 24 h after pretreatment with streptomycin results in mild (<10%) weight loss in the first 5 days after infection with subsequent full recovery by day 9 (FIG. 25 a). In contrast, homozygous P-2−/− mice develop bloody diarrhea associated with progressive weight loss to day 5 or 6 at which time they were euthanized for analysis and to prevent further suffering. Even challenge with only 10² salmonellae caused progressive disease in P-2−/− mice and lethality. P-2+/− littermates had more severe disease than P-2+/+ littermates but recovered (FIG. 25a).

Severe weight loss of S. typhimurium infected P-2−/− mice was associated high bacterial titers in blood and dissemination to multiple organs (FIG. 25b). Histopathological examination showed complete dissolution of the epithelial barrier in the small and large intestine and massive cellular infiltration that, however, was unable to clear the bacteria (not shown).

Rejection of Salmonella by IFN-γ activated peritoneal exudate macrophages from P-2−/−mice (P-2−/− PEM) was also analyzed in vitro. PEM were infected for one hour, washed and then incubated for times indicated, lysed and CFU determined. Salmonella replicated to high numbers within P-2−/− PEM. In contrast, in P-2+/+ PEM their number was reduced by about 50% in 4 h and then held steady. Heterozygous P-2+/− PEM only delayed Salmonella replication. For comparison, P-2 knock down with siRNA in wild type PEM resembles to P-2−/− PEM validating the knock down technique (FIG. 25c).

### Interferons Induce Perforin-2 in all Cells and Enable Bactericidal Activity

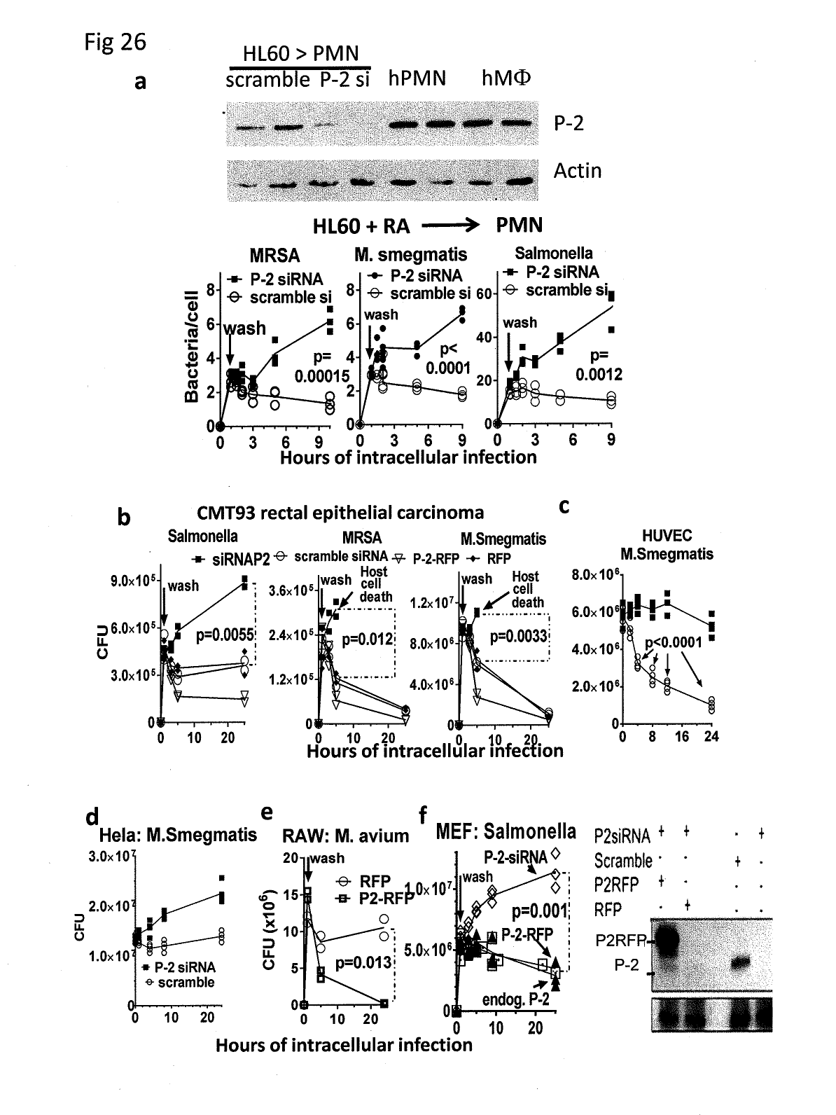

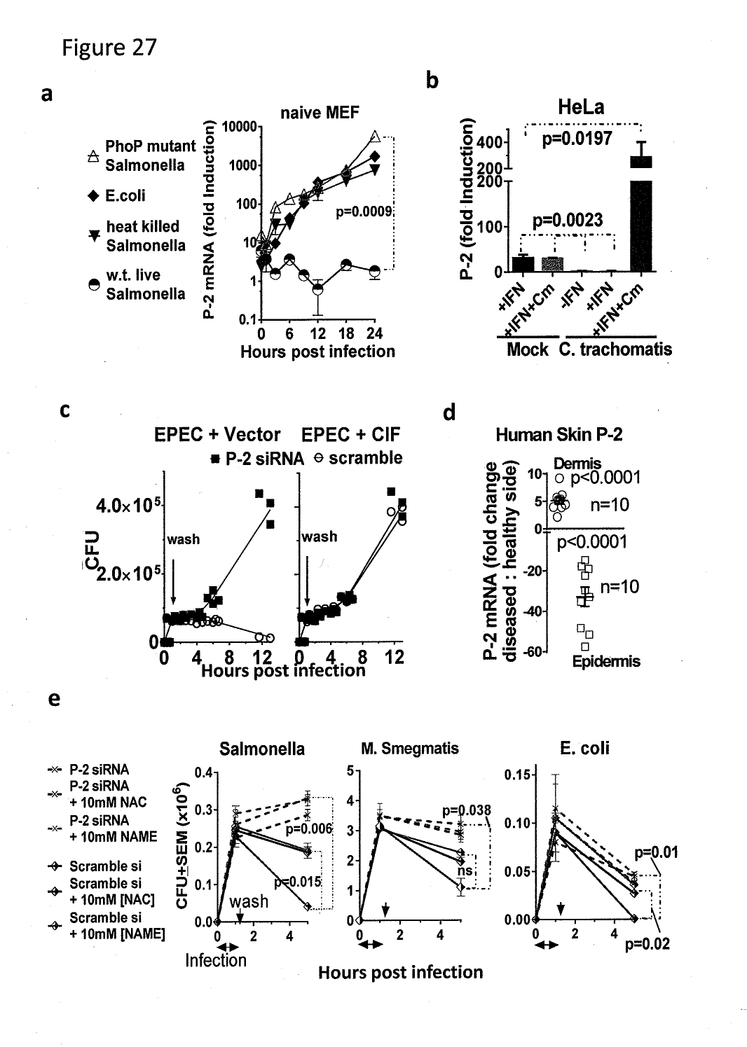

The rapid spread of Salmonella in P-2−/− mice suggested that none of the cells in the P-2−/− mouse were able to stop bacterial dissemination. We therefore investigated which cell types expressed P-2 constitutively or can be induced to express P-2. Table 2 summarizes the data. PMN, macrophages, dendritic cells and microglia constitutively express Perforin-2 mRNA and protein which is further upregulated by IFN-γ and LPS (not shown). Human primary keratinocytes and keratinocytes from tissue samples taken from the edge of chronic wounds (FIG. 27) likewise express P-2 constitutively. All other cells and cell lines tested express P-2 mRNA only after treatment with IFN-α-β or -γ. All cells expressing P-2 are able to control intracellular bacterial infection, which however is abolished when P-2 is knocked down with siRNA. Human PMN and macrophages express P-2 protein constitutively (FIG. 26a). Knock down of Perforin-2 in PMN, induced by retinoic acid from HL60, significantly inhibits bactericidal activity and enables intracellular replication of MRSA, M. smegmatis and S. typhimurium (FIG. 26a). Likewise P-2 knock down significantly inhibits killing of intracellular bacteria in intestinal epithelial cells (rectal carcinoma CMT93) (FIG. 26b), human endothelial cells (HUVEC) (FIG. 26c), and human cervical carcinoma epithelial cells HeLa) (FIG. 26d). Elevated expression of P-2 by P-2-GFP transfection increases bactericidal activity which is important for eliminating Mycobacterium avium (FIG. 26e). Endogenous P-2, knocked down with siRNA specific to the 3′-untranslated region of P-2, is complemented by transfection with P-2-GFP or P-2-RFP and fully reconstitutes bactericidal activity in MEF (FIG. 26f) or phagocytic cells (not shown). Cumulatively, our data show that P-2 is expressed or can be induced ubiquitously and is required to kill or inhibit intracellular replication of at least the three types of bacteria examined.

### Bacteria can Block Perforin-2 Induction and Activation

The potent bactericidal activity of P-2 suggests that intracellular bacteria must evolve strategies to evade Perforin-2. In principle, bacteria could block Perforin-2 transcription in non-phagocytic cells, cause down regulation in constitutively Perforin-2 expressing cells or interfere with Perforin-2 activation and polymerization. Infection of naïve MEF with the laboratory strain E. coli K12 results in rapid induction of P-2 mRNA and subsequent killing of the intracellular bacteria. Unlike E. coli, live wild type S. typhimurium does not cause P-2 mRNA induction in MEF (FIG. 27a). In contrast, heat killed or PhoP mutant Salmonella induce P-2 mRNA in MEF to a similar extent as E. coli suggesting that S. typhimurium actively suppresses P-2 induction. Similarly, infection of HeLa cells with the obligate intracellular bacterium Chlamydia trachomatis does not induce P-2 expression. Moreover, P-2 induction by exogenous IFN is actively suppressed via a mechanism that requires de novo chlamydial protein synthesis (FIG. 27b). EPEC can block endocytosis but phagocytic cells can overcome this inhibition (FIG. 27c). Endocytosed EPEC are protected from Perforin-2 only when they carry the Cif plasmid (FIG. 27d) that encodes a deamidase that inactivates NEDD8, a ubiquitin-like molecule. NEDD8 is required for the activation of cullin-ring ubiquitin E3-ligases (CRL5) that participate in many fundamental cellular pathways, including NFκB activation. Neddylation is carried out by the NEDD8 specific E2-ligase Ubc12. The cytoplasmic domain of P-2 interacts with Ubc12 in the yeast two hybrid system and is co-immunoprecipitated with P-2 (supplemental FIG. 27) suggesting that Ubc12 mediated neddylation of a CRL is required for Perforin-2 mediated killing and that CIF blocks this step.

As shown in Table 2, keratinocytes constitutively express Perforin-2 mRNA and protein suggesting a contribution to the barrier function of skin against infection. We examined the level of P-2 expression in the dermis and epidermis of 10 patients with non-healing chronic skin ulcers with high bacterial burden. Excisional surgical debridement of the wound and wound edges was performed as part of standard care. Subsequently, the excised skin was divided into wound-adjacent and normal skin-adjacent halves, separated into dermis and epidermis and P-2 mRNA levels quantitated by TaqMan-PCR. Perforin-2 mRNA levels were approximately 35 fold lower in epidermal cells on skin adjacent to the chronic ulcer compared to normal skin-adjacent cells. In contrast, in the underlying dermis the ratio of P-2 mRNA was in the opposite direction with P-2 mRNA levels four fold higher near the wound (FIG. 27d). The data show that in chronic skin ulcers with high bacterial burden P-2 levels are affected and may be associated with delayed healing.

### Perforin-2 Enhances the Bactericidal Activity of Reactive Oxygen and Nitrogen Species

The current paradigm suggests that intracellular bacteria are killed by reactive oxygen and nitrogen species and by fusion of the phagocytosed bacteria with the lysosome. Here we analyze the bactericidal efficiency of each effector pathway and their synergism. We blocked each effector individually and determined intracellular survival and replication or killing of intracellular S. typhimurium and M. smegmatis. These experiments were carried out with intestinal epithelial cells (CMT93) (not shown) and in IFN-γ activated PEM (FIG. 27e) with virtually identical results. First, we ascertained that IFN-γ activated and LPS stimulated PEM produced ROS and NO and that this production is not affected by P-2 siRNA knock down. We also ascertained that the ROS and NO inhibitors used were specific and active in blocking ROS or NO (not shown). PEM are able to kill about 90% of intracellular Salmonella in the first four hours after infection when all three effectors were active in scramble siRNA transfected cells (FIG. 27e, solid lines). Knock down of P-2 completely abolished the bactericidal activity against Salmonella and allowed their intracellular replication (FIG. 27e, dashed lines). Additional inhibition of ROS and NO with NAC and NAME had no further effect. In the presence of P-2 (scramble siRNA, blue and green solid lines) inhibition of ROS or NO also significantly diminished the bactericidal activity but in the presence of P-2 bacterial replication was still blocked. M. smegmatis is more resistant to killing by bactericidal effectors within 4 hours. Nonetheless, Perforin-2 knock down significantly inhibited killing of M. smegmatis and additional blockade of ROS or NO had no further effect. Blockade of ROS or NO without P-2 knock down had only limited, non-significant effects on bactericidal activity against Mycobacteria. In contrast, non-pathogenic laboratory E. coli K12 is sensitive to intracellular killing by ROS and NO even without P-2 although significantly less efficient than in the presence of P-2. The data indicate that Perforin-2 synergizes with reactive oxygen and nitrogen species and with lysozyme as shown previously and that bacteria are differentially susceptible to individual effectors.

### Perforin-2 Translocates to the Bacterium Containing Vacuole.

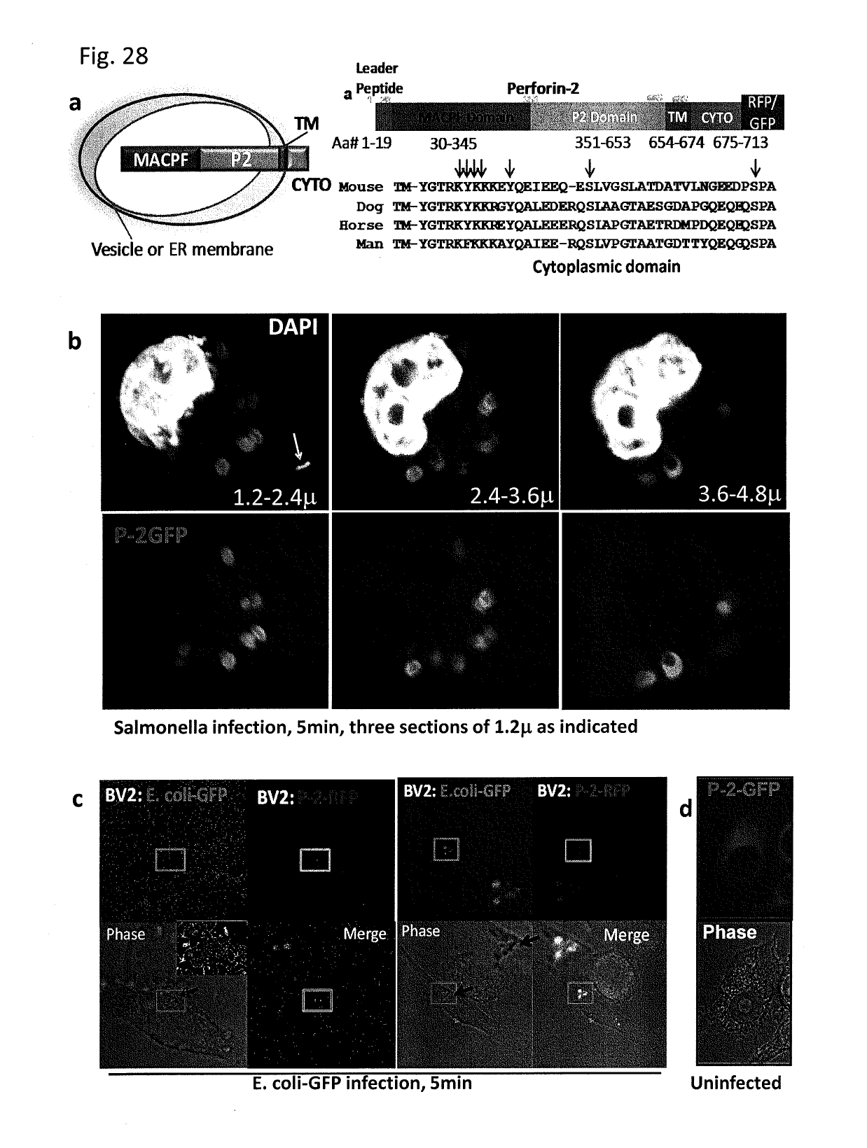

In the absence of infection, Perforin-2 is stored embedded in the membranes of a perinuclear vesicle compartment (FIG. 28b). Therefore, in order to kill, Perforin-2 must be transported to the site of bacterial infection. Perforin-2 has a short, highly conserved cytoplasmic domain which can interact with cytoplasmic proteins that trigger P-2 translocation and polymerization (FIG. 28a). Mutation of Y to F (indicated by red arrow in FIG. 28a) blocks the bactericidal activity of P-2 suggesting an important function for P-2 activation (not shown).

We tested Perforin-2 translocation to the bacterium containing vacuole by confocal microscopy. In these experiments endogenous Perforin-2 in BV2 cells was knocked down and reconstituted with P-2-GFP by transient transfection. BV2 were infected with Salmonella that actively invade cells by inducing endocytosis (FIG. 28b). In this experiment bacteria are imaged by staining of bacterial DNA with DAPI (shown in white for better visibility) and colocalization is imaged with P-2-GFP. Five minutes after infection several salmonellae are already endocytosed and apparently lysed as suggested by a ‘cloud’ of DAPI staining of released DNA endocytosed salmonellae. Three 1.2μ thick slices show the number lysed salmonellae (8) and the size of diffuse DNA containing endocytic vesicles revealed by DAPI and P-2-GFP. One intact salmonella stained by DAPI (arrow) is still seen outside the cell as rod like structure in the bottom section (left panels) but not in the top two 1.2 g sections. We also analyzed transiently P-2-RFP transfected BV2 by infection with GFP-marked E. coli for P-2 translocation. Within five minutes of infection P-2 was found on the membrane of the bacterium containing vacuole and on the bacterium (FIG. 28b). The fluorescence intensity suggests that Perforin-2 is highly enriched at this site suggesting specific targeting mechanisms. The bacteria appear fragmented and have released GFP detectable as diffuse fluorescence within the vacuole. The data indicate that Perforin-2 translocation to the bacterium containing vacuole is complete within minutes of infection and is associated with fragmentation of bacteria inside the vacuole and release of their DNA consistent with P-2-attack and pore-formation on bacterial cell walls.

### Perforin-2 Forms ˜90 Å Pores in Bacterial Cell Walls

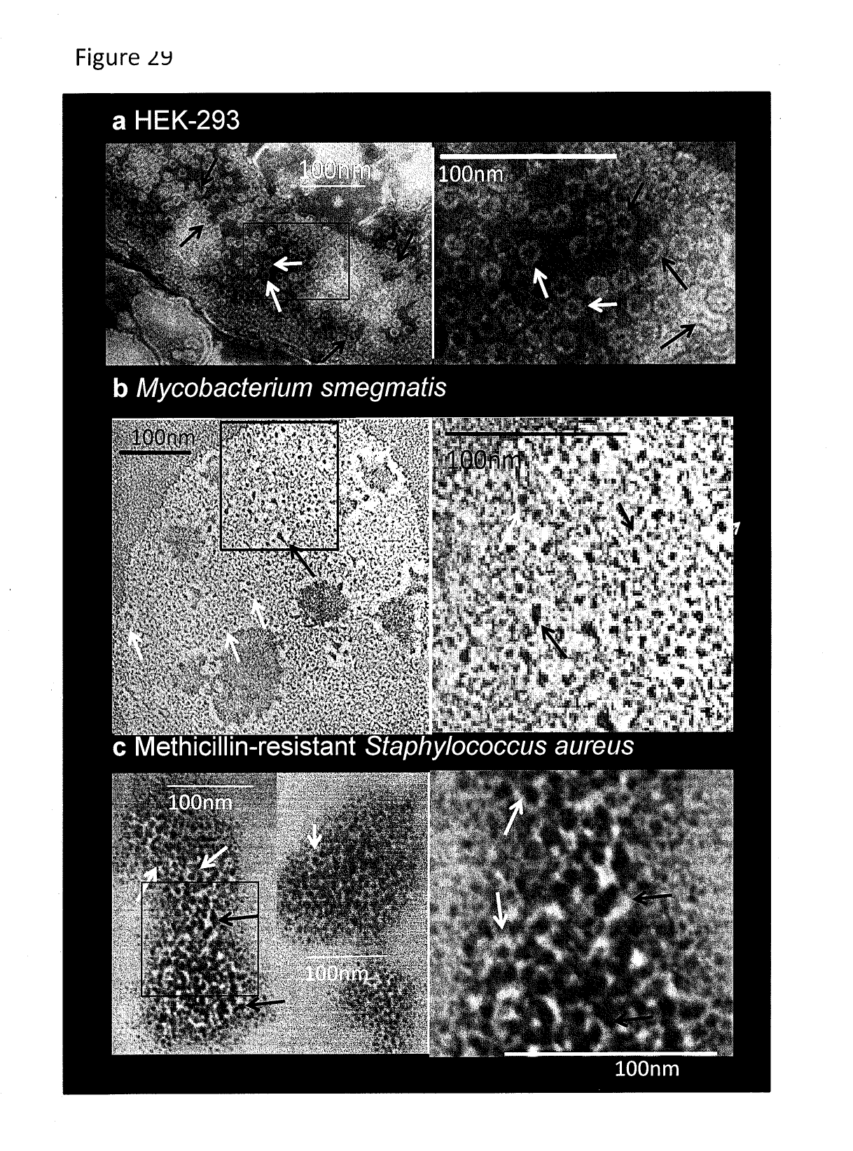

The finding that Perforin-2 accumulates within minutes of infection on membranes enclosing bacteria with its effector domain pointing towards the lumen suggests that Perforin-2 may deliver the lethal hit to bacteria by polymerizing in their cell wall and creating water filled pores that enhance the penetration of other bactericidal factors, similar to the cytotoxic mechanisms of complement C9 and Perforin-1. We determined the ability of Perforin-2 to form pores which has not previously been reported. We reasoned that Perforin-2 monomers similar to C9 or Perforin-1 monomers are not cytotoxic and that pore formation requires P-2 polymerization via specific interaction of the cytoplasmic domain of P-2 with as yet unknown signaling proteins. The cytoplasmic domain of Perforin-2 contains a conserved RKYKKK (SEQ ID NO:4) sequence (FIG. 28a, blue arrows) next to the transmembrane domain which may function as proteolytic cleavage site to trigger P-2 polymerization on the opposite side of the membrane. This was tested by purifying Perforin-2-GFP bearing membranes from P-2-GFP transfected HEK293 cells by differential centrifugation and treating them briefly with low levels of trypsin to cleave the cytoplasmic domain (not shown). Electron microscopic examination of trypsin treated Perforin-2-GFP-membranes show typical transmembrane pores with an average inner diameter of 85-95 Å (FIG. 29a, white arrows). In addition half-ring, 8-shaped, and more irregular structures formed by fusion of Perforin-2 during polymerization (black arrows) are consistent with a polymerization process that continues until termination by ring closure. As with C9 and Perforin-1, ring-closure may be blocked by steric hindrance of membrane proteins. Untransfected cells did not have such pores (not shown). These data directly show that Perforin-2 is indeed a pore-forming transmembrane protein potentially able to form pores on bacterial cell walls inside vacuoles. Since Perforin-2 is a membrane protein with the MACPF domain pointing into the lumen of a vesicle, pore formation will occur in membranes touching the MACPF domain inside the vesicle.

To test that P-2-pores are assembled on killed bacteria, we obtained intracellular bacteria from IFN activated, Perforin-2-sufficient MEF three hours after infection by hypotonic non-ionic detergent lysis. Bacterial cell walls, unlike phospholipid bilayers, are resistant to lysis by mild detergents and are obtained by centrifugation and imaged by electron microscopy. On M. smegmatis cell walls many clustered pores of about 90 Å are seen (FIG. 29b white arrows), frequently with irregular structures (black arrows). Steric hindrance by rigid M. smegmatis cell walls appears to frequently interfere with ring closure of the Perforin-2-polymer resulting in more irregular polymers. Surface staining by negative stain of M. smegmatis cell walls outside the pores is minimal, consistent with the known hydrophobicity of the cell wall of mycobacteria. The cell walls of S. aureus in contrast appear more hydrophilic than mycobacterial cell walls by allowing the negative stain to adhere. S. aureus cell walls are more rigid than phospholipid membranes, judging by the irregularity (black arrows) and varying size of Perforin-2 pores (FIG. 29c).

### Materials and Methods

### Plasmid Constructs

The complete coding region of murine mpeg1 cDNA was constructed from several EST clones and inserted into the pEGFP-N3 plasmid (Clontech). Monomeric RFP (R. Flavell, Yale) was cloned in place of GFP for use in some experiments.

### Cell Lines and Primary Cells

RAW264.7, J774, HL-60, and HEK-293 cell lines were obtained from ATCC. BV2 microglial cell line was a gift from Dr. J. Bethea, University of Miami. All cells were cultured at 37° C. in a humidified atmosphere containing 5% CO₂ following ATCC recommendations. HL-60 were differentiated toward PMN phenotype using retinoic acid as previously described. Murine primary macrophages were obtained from thioglycolate-elicited peritoneal or bone marrow as previously described. Human macrophage and PMNs were isolated from fresh healthy donor PBMC. Human macrophages were differentiated from monocytes as described previously and human PMN were isolated as previously described. Murine embryonic fibroblasts (MEFs) were isolated as previously described.

### Bacterial Strains:

S. typhimurium strain LT2Z (gift from Dr. G. Plano, University of Miami), K12 E. coli, and methicillin-resistant S. aureus (gift from Dr. L. Plano, University of Miami) were grown in Luria broth (LB).

M. avium intracellulare (gift from Dr. T. Cleary, University of Miami), and M. smegmatis (ATCC) were grown in Middlebrook 7H9 broth.

### Chemicals and Cytokines

Lipopolysaccharide (LPS) was purchased from Sigma and used at a final concentration of 1 ng/ml. Recombinant human and murine IFN-γ was purchased from Peprotech and used at a final concentration of 100 U/ml. N-acetyl cystine and L-NAME were both purchased from Sigma.

### Antibodies

Rabbit anti-Mpeg1, human anti-Mpeg1, and anti-GFP polyclonal antibodies were obtained from Abcam and used for western blot analysis. Rabbit anti-Perforin-2 (cytoplasmic domain) antiserum was produced and obtained by 21^{St} Century.

qRT-PCR

RNA was extracted from cells following RNeasy (Qiagen) instructions. One ttg of RNA was converted to cDNA using QuantiTect Reverse Transcription kit (Qiagen) following the supplier's protocol. qRT-PCR was performed using Taqman® Gene Expression Assays (Applied Biosystems) for murine mpeg1, GAPDH, and β-Actin. For human tissue, human mpeg1, GAPDH, and β-Actin probes were utilized. All assays were performed on the Applied Biosystems 7300 PCR platform.

### Gentamicin Protection Assay

Intracellular bactericidal activity was adapted from. In brief, bacteria were grown at 37° C. with shaking for 16-18 hours in Luria broth (LB) (S. typhimurium, S. aureus, and E. coli) or 24 hours in Middlebrook 7H9 broth (Mycobacteria) prior to infection. For S. typhimurium, E. coli, and S. aureus the culture was then diluted 1:33 in LB and grown for another 3 hours to allow the bacteria to enter log phase and for Salmonella to induce the invasive phenotype. Eukaryotic cells were transfected following Lonza's optimized protocol for the respective cells, and plated into 12-well plates post-transfection. HL-60 cells differentiated with RA were not stimulated; RAW264.7 cells were stimulated for 14 hours with LPS (1 ng/ml) and IFN-γ (100 U/ml) to differentiate toward a macrophage lineage; all other cells were stimulated with species-specific IFN-γ (100 U/ml). Cells were infected at a multiplicity of infection (MOI) between 10 and 60 for 30 minutes (S. typhimurium) or 1 hour (S. aureus, E. coli, and M. smegmatis) in a 37° C., 5% CO₂ incubator. After infection, cells were washed twice with ice-cold PBS and fresh medium containing 50 μg/ml gentamicin was added. After 2 hours, the medium was changed to decrease the concentration of gentamicin to 5 μg/ml. At indicated time points, cells were washed with PBS, lysed using 1% Igepal in ddH₂O, diluted and plated in triplicates on LB agar plates (S. typhimurium, S. aureus, E. coli) or Middlebrook 7H11 plates (Mycobacteria) and CFU determined after colony growth.

### Gentamicin-Free Intracellular Bacterial Killing Assay

The gentamicin protection assay was modified to create the gentamicin free intracellular bacterial killing assay. The modifications to the above included plating eukaryotic cells to achieve a confluence of 90-100% on infection, and decreased multiplicity of infection (MOI) to between 5 and 15. Invasion times were left unchanged with 30 minutes for S. typhimurium and 1 hour for S. aureus, E. coli, and M. smegmatis and with infection occurring in a 37° C., 5% CO₂ incubator. To ensure maximal elimination of extracellular bacteria, wash steps were altered such that cells were washed twice with ice-cold PBS, trypsinized to help eliminate extracellular bacterial attachments, and washed an additional 3 times with ice-cold PBS. Every four hours, media was removed and the cells were washed twice with PBS and then fresh media added back. At indicated time points, cells were washed three times with PBS and lysed utilizing 1% Igepal in ddH₂O, diluted and plated in triplicates on LB agar plates (S. typhimurium, S. aureus, E. coli) or Middlebrook 7H11 plates (Mycobacteria) and CFU determined after colony growth.

### RNA Interference

For murine cells, three Perforin-2-specific chemically synthesized 19-nucleotide siRNA duplexes were obtained from Sigma. Two siRNAs were complementary to the 3′ UTR of Perforin-2 and the third complementary to the coding region. The sequences were as follows: CCACCUCACUUUCUAUCAA (SEQ ID NO:1), GAGUAUUCUAGGAAACUUU (SEQ ID NO:2), and CAAUCAAGCUCUUGUGCAC (SEQ ID NO:3). A scramble siRNA was also generated to serve as a control to the reaction. For human cells, three human Perforin-2-specific silencer select siRNAs were purchased from Ambion (Invitrogen) Silencer Select #s61053, s47810, s61054. Silencer select negative control #2 from Ambion (Invitrogen) was also used. Transfection of siRNA into all cells was carried out using Amaxa Nucleofector System (Lonza) according to manufacturer's instructions. All transfections were carried out using 1-4×10⁶ cells, a final concentration of 300 nM siRNA (Perforin-2-specific siRNAs were pooled) and 2 μg of plasmid DNA where indicated. Immediately after transfection, cells were plated in antibiotic-free complete medium.

### Negative Staining Transmission Electron Microscopy

Eukaryotic Cell Membranes:

membranes were isolated from stably transfected Perforin-2-GFP HEK-293 cells by N₂-cavitation and differential centrifugation. Membranes were resuspended in a small volume of neutral Tris-buffered saline, treated with 100 μg/ml trypsin for 1 hour at 37° C., washed and negatively stained with 5% neutral Na-phosphotungstate for 30 seconds. Images were taken at 52,000-fold initial magnification on a Phillips CM10 transmission electron microscope.

Bacterial Membranes:

MEF were stimulated for 14 hours with IFN-γ (100 U/mL) and infected with the indicated bacterial strains at a multiplicity of infection of 30 for 5 hours. Prokaryote membranes were harvested through lysing MEFs with 1% Igepal in ddH₂O. The lysate was centrifuged at 200 g for 10 minutes to pellet intact bacteria, intact bacteria were subsequently sheared with a polytron to disrupt intact bacteria and separate out the membranes. The resulting pellet was treated with 100 μs/ml trypsin for 1 hour at 37° C., sedimented and resuspended in minimal ddH₂O and negatively stained with 3% uranyl formate for 30 seconds. Images were taken between 52,000 to 168,000-fold initial magnification on a Phillips CM10 transmission electron microscope.

### Confocal Microscopy

For Perforin-2-GFP localization, RAW264.7 cells were transiently transfected with Perforin-2-GFP and stimulated overnight with LPS (1 ng/ml) and IFN-γ (100 U/ml) in glass bottom dishes with No. 1.5 covergiass (MatTek Corp). Cells were washed once with PBS and organelles were labeled. For endoplasmic reticulum (ER) labeling, we used ER-Tracker™ Blue-White DPX (Invitrogen) at a working concentration of 1 μM for 30 minutes at 37° C. For all other stains, transfected cells were fixed with 3% paraformaldehyde (PFA) for 15 minutes at room temperature, permeabilized with 0.5% saponin, blocked with 10% normal goat serum and incubated with primary and secondary antibodies. Anti-CD107a (LAMP-1) (BD Pharmingen), anti-CD11b (BD Pharmingen), anti-golgin97 (Invitrogen), anti-EEA1 (Calbiochem), anti-GM130 (BD biosciences), and Hoechst 33258 (Invitrogen) were used to identify cellular organelles. Secondary antibodies were all raised in goat. Specimens were kept in PBS and imaged at room temperature on a Leica SP5 inverted confocal microscope with a motorized stage and Leica DFC495 camera. Images were analyzed using Leica application suite advanced fluorescence software and deconvolution processing was applied.

For Perforin-2-RFP and E. coli localization imaging, BV2 cells were co-transfected with Perforin-2-RFP and siRNA targeting the 3′UTR of endogenous Perforin-2 and stimulated overnight with IFN-γ (100 U/ml) after adhering to coverslips. Cells were infected at an MOI of 100 by GFP-E. coli for 5 minutes at which point the cells were washed twice with PBS and fixed with 3% PFA for 15 minutes at room temperature. Individual coverslips were mounted to slides utilizing Vectashield Mounting Media and imaged at room temperature on a Leica SP5 inverted confocal microscope with a motorized stage and Zeiss LSM710 inverted confocal microscope. Images were analyzed using Leica application suite advanced fluorescence software.

Leica SP5 confocal microscope used a plan-apochromat 63×/1.4NA objective lens, 405, 488, 561 nm lasers (633 nm laser if you included Cy5 or Alexa Fluor 647). Pixel size was set to 60 nm, to obtain 2D Nyquist sampling (diffraction limit 214 nm for 500 nm light).

Zeiss LSM710 confocal microscope used a plan-apochromat 63×/1.4NA objective lens, 405, 488, 561 nm lasers (633 nm laser if you included Cy5 or Alexa Fluor 647). Pixel size was set to 60 nm, to obtain 2D Nyquist sampling (diffraction limit 214 nm for 500 nm light).

### Example 5

### Define Function of RASA2/GAP1M P-2-Interaction in Clearance of Intracellular Bacteria

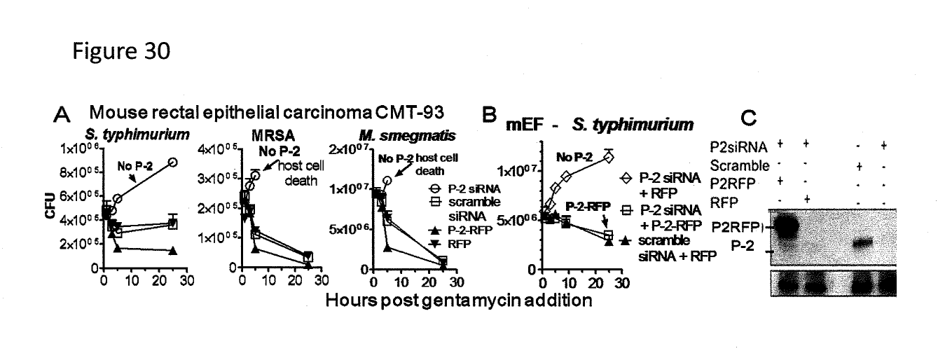

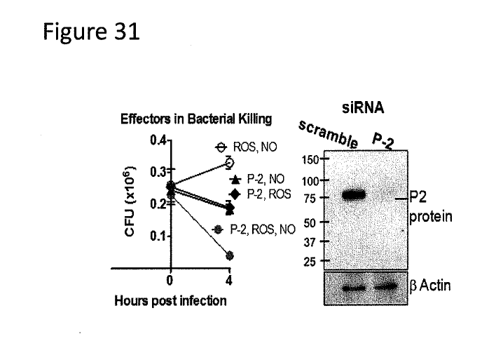

P-2 siRNA knock down of P-2 in macrophages, microglia, mEF, HeLa and other freshly explanted cells or cell lines blocks killing and clearance of intracellular bacteria, including pathogenic Salmonella typhimurium, Methicillin Resistant Staphylococcus Aureus (MRSA), Mycobacterium smegmatis (FIG. 30 A) and Mycobacterium avium (not shown) determined in the gentamycin protection assay and also in the absence of antibiotics (not shown). In FIG. 30 rectal epithelial cells (A) or mouse embryonal fibroblasts (mEF) (B), were transiently transfected 24 h before infection with a pool of 2 siRNAs specific for the 3′UTR of P-2 or with control scrambled siRNA which knocks down P-2 RNA and protein (FIG. 31C); for P-2 complementation (B) the cells are co-transfected with P-2 siRNA and knock-down resistant P-2-RFP (not expressing the 3′UTR of endogenous P-2) or with control RFP. P-2-RFP is a fusion protein tagged at the C-terminus of P-2 with monomeric DsRed (RFP). Transfected P-2-RFP is fully active in bacterial killing (FIG. 30 B,C). At −16 h IFN-γ is added to induce P-2 mRNA (which is suppressed when P-2 siRNA is present). At time 0 the cells are infected for 30-60 min with pathogenic MRSA, Salmonella typhimurium or Mycobacterium smegmatis at a multiplicity of infection (MoI) of 30-50 bacteria per cell. After intracellular infection, extracellular bacteria are washed away in PBS and the cells replated in gentamycin to prevent extracellular replication of bacteria. The host cells are lysed with mild detergent (NP40) immediately after infection and at several time points later and surviving bacteria enumerated by replicate colony forming assays. Mild detergents lyse host cell membranes but not bacterial cell walls.

In rectal epithelial cells (FIG. 30) control-scramble siRNA transfection does not affect P-2 and does not inhibit the ability of the epithelial cells to kill intracellular bacteria while P-2 siRNA blocks killing and allows intracellular replication killing the host cell. P-2-RFP expression in addition to endogenous P-2 increased bacterial killing (FIG. 30 A). When endogenous P-2 is knocked-down, complementation with P-2-RFP restores killing (FIG. 30 B). As would be expected for an innate defense mechanism P-2 is able to kill bacteria with very diverse outer cell walls such as Gram-negative Salmonella, Gram-positive Staphylococci and acid fast Mycobacteria.

ROS and NO are effectors known to kill intraphagosomal bacteria. We therefore directly compared the effect of blocking either P-2 or ROS (with NAC) or NO (with NAME) on the bactericidal activity of IFN-γ activated peritoneal macrophages for Salmonella typhimurium, Mycobacteria and MRSA (FIG. 31). Data are similar for all bacteria and are shown for S. typhimurium In FIG. 31. When P-2, ROS and NO are not inhibited there is excellent intracellular killing (line labeled in FIG. 31 P-2, ROS, NO). In the absence of P-2, bacteria replicate despite ROS and NO. Inhibition of ROS with NAC or NO with NAME slows down the killing activity, suggesting that both ROS and NO enhance P-2 mediated killing.

The data show that P-2 is critical for killing of intracellular pathogenic bacteria, because in the absence of P-2 the bacteria replicate in the cell (FIG. 30 and FIG. 31, red ROS, NO curve). This is a critical difference. Continued replication of bacteria kills the host cell and, in vivo, would spread the infection to other cells. We have data showing that all phagocytic and non-phagocytic human and mouse cells and cell lines tested to date use Perforin-2 to eliminate intracellular bacteria (Table 2). Non-phagocytic cells use autophagy to clear intracellular bacteria. Our data indicate the P-2 may cooperate with autophagy to deliver the lethal hit to bacteria.

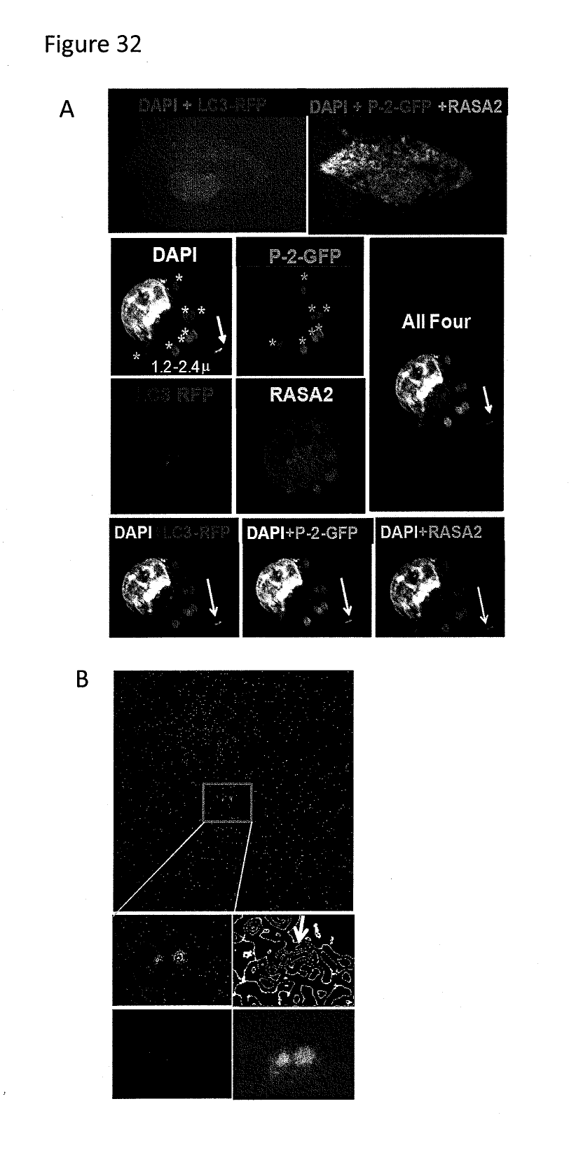

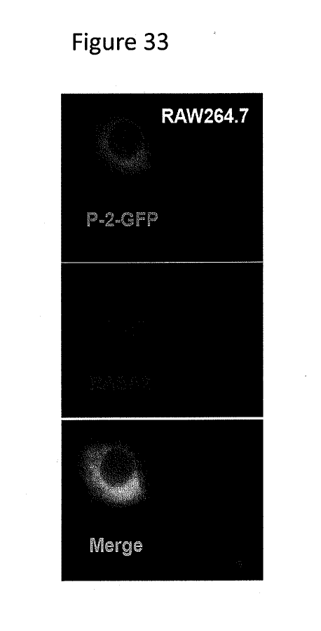

P-2-GFP is a transmembrane protein localized in resting BV2 (FIG. 32a, two top panels) and RAW cells (FIG. 33) in perinuclear membrane-vesicle compartments. P-2-GFP (green) colocalizes in part with orange RASA2 (seen as yellow) (FIGS. 32a and 33). LC3-RFP (red) is more homogeneous than RASA2 or P-2. The nucleus is shown in blue by DAPI staining.

Salmonella actively invade cells within 5 minutes by triggering endocytosis with the type III secretion system. Bacteria can be visualized by DAPI (DNA) staining shown in white (for better visibility) in all lower panels in FIG. 32a. Intact extracellular Salmonella have rod like appearance (white arrows). After 5 minutes incubation of IFN activated BV2 (macrophage-derived microglia) with Salmonella typhimurium, several Salmonella are seen endocytosed by the cell and have already released their DNA (asterisks in DAPI and P-2-GFP panels, compare morphology to extracellular Salmonella indicated by arrow) most likely because they have been killed by P-2-GFP. The internalized bacteria are in a vacuole whose membrane stains with P-2-GFP, RASA-2 and LC3-GFP. In this experiment endogenous P-2 had been knocked down with siRNA and reconstituted with transfected P-2-GFP. The transfection mix also contained LC3-RFP. Similar data are shown for E. coli (FIG. 32b). These data directly support that P-2 mediates killing of intravacuolar bacteria and that RASA2 participates in P-2 translocation and that LC3 as marker for autophagy implicates autophagy in P-2 mediated killing of intracellular bacteria.

These data were collected with phagocytic cells; we now wish to determine these mechanisms in non-phagocytic cells which rely on autophagy for bacterial clearance. The studies are likely to link P-2 to autophagy and provide a molecular mechanism for the lethal hit in autophagy.

Endocytosed bacteria initially are close to the plasma membrane where autophagy is initiated within minutes of infection. Rab5 on the bacterium containing vacuole recruits vps34, the autophagy associated PI3-kinase. Generation of PI3P and PI(3,4,5)P3 is required for maturation of the vacuole to phagosomes and autophagosomes, respectively. We propose that RASA2 binds to PIP3 on the vacuole by translocation from perinuclear membranes. RASA2 may provide the molecular switch that is typical for RasGTPases function in a large number of signaling pathways. RASA2 could provide this switch for P-2-vesicle transport and translocation to the vacuole. Rab5 which recruits vps34 to the vacuole may also transport P-2 but other mechanisms are also possible. Imaging analysis of Perforin-2 and GAP1M/RASA2 in mEF and BV2 microglia. To validate in cells RASA2 interacting with the cytoplasmic domain of P-2 in the yeast two hybrid system, we determined localization of the two proteins in RAW macrophages. Macrophages express P-2 constitutively. To determine colocalization we generated a P-2-GFP fusion protein, fused via a linker to the C-terminus of the cytoplasmic domain of P-2. P-2-GFP is functional in killing of intracellular bacteria in complementation assays (FIG. 30 B, FIG. 31). P-2-GFP is localized in perinuclear membrane vesicles of RAW cells (FIG. 33, top) similar to RASA2 (FIG. 33, center). When merging the images P-2-GFP and RASA2 appear colocalized on a large number of perinuclear vesicles (FIG. 33, bottom). We propose that RASA2 translocates to the bacterium containing vacuole upon vps34 phosphorylation.

We will determine RASA2 localization and translocation upon bacterial infection. RASA2 is expressed in most or all cells constitutively (albeit at different levels) and its localization can be determined by specific antibodies (see FIG. 33). After having determined kinetics of RASA2 translocation following infection, we will transfect with P-2-GFP (or -RFP) as in FIG. 32 and measure P-2 translocation and its relation to RASA2 translocation by two and three color (using fluorescent-labeled bacteria) imaging analysis.

Rab5 recruits the PI3-kinase vps34 from its cytoplasmic and perinuclear localization to the bacterium containing vacuole. Vps34 via phospatidyl-inositol-phosphorylation may provide the inositol-phosphate code for RASA2 translocation. We will determine whether the vps34 inhibitor 3-MA blocks translocation of RASA2 and P-2. We will determine the effect of bacterial infection on the intracellular location of RASA2 in BV2, naïve mEF or IFN-preactivated mEF. Intracellular bacteria manipulate vesicle transport to enhance their intracellular survival. Salmonella increases PI3P generation for the generation of a Salmonella containing vacuole (SCV) allowing survival while Mycobacteria decrease PI3P generation to prevent maturation of the vacuole and fusion with lysosomes. Different bacterial species therefore may be able to manipulate RASA2 translocation with the potential consequence of delaying or blocking P-2 recruitment to the vacuole.

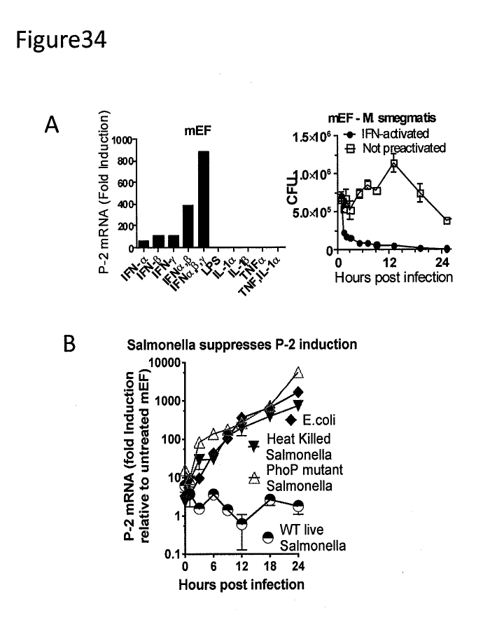

We will compare translocation in BV2 microglia with naïve (resting) mEF and IFN-preactivated mEF to determine whether bacterial endocytosis triggers similar responses and whether pre-activation of mEF with IFN changes the kinetics or quality of the response with regard to RASA2 translocation. IFN induces a large number of genes that may include factors in naïve non-phagocytic cells that are required for P-2's function in attacking intracellular bacteria. As shown in FIG. 34a (left panel) IFN-pre-activated mEF have killed most M. smegmatis within 2 h post infection while naïve mEF begin killing only by 12 h. This kinetics of RASA2 translocation may reflect this delay as well.

We will also knock down Rab5 and vps34 and determine the effect on RASA2 translocation and intracellular killing of bacteria in the CFU assay.

We anticipate that IFN-pre-activated mEF will be comparable to phagocytic BV2 in the mechanisms of P-2 translocation and activation for killing. If RASA2 recruitment and translocation is required, it is expected to be similar in activated mEF and BV2. In naïve mEF the recruitment of RAS2A2 and P-2 may not be synchronized because P-2 must first be induced. Live but not dead Salmonella blocks induction of P-2 mRNA (FIG. 34b) in mEF providing a mechanism of protection of Salmonella in non-phagocytic cells. In separate studies we are studying the virulence genes of S. typhimurium (see PhoP as an example, FIG. 34b) that are responsible for P-2 suppression. We have also data that Chlamydiae suppress P-2 induction (data not shown). These findings support our proposal that bacteria must suppress P-2 if they want to set up intracellular residence.

To obtain further insight into the early events after bacterial infection we will also image Rab5 and vps34 which we expect to translocate to the bacterial vacuole.

Analysis of the Effect of RASA2 siRNA Knock Down on Killing of Intracellular Bacteria.

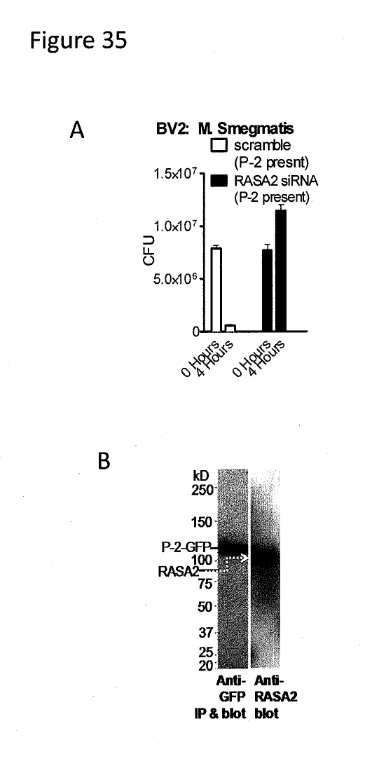

Blockade of any molecule that is required for P-2 induction, recruitment, translocation, activation or polymerization is expected to also block killing of intracellular bacteria. If RASA2 is required for P-2 translocation, activation and/or killing, then its knock-down should inhibit killing of intracellular bacteria. This was tested in initial experiments in BV2 microglia cells killing intracellular M. smegmatis (FIG. 35a). RASA2 knock down completely blocked intracellular killing similar to P-2 knock down and allowed intracellular replication suggesting that RASA2 is important for killing of intracellular bacteria.

In addition Rab1, Rab5, Rab7 and Rab11 and vps34 will be knocked down and the effect on bacterial killing determined in the gentamycin protection assays.

The requirement for RASA2 will be further tested in IFN pre-activated and un-activated (naïve) mEF and rectal epithelial cells CMT93 by measuring RASA2 message levels by qPCR. We will also analyze intracellular killing or survival of MRSA, M. smegmatis and S. typhimurium following siRNA knockdown in the gentamycin protection-CFU-assay.

Biochemical Interaction of P-2 with RASA2 by Coimmunoprecipitation.

We will immunoprecipitate BV2- and mEF-lysates with anti RASA2 monoclonal antibody, separate by SDS-PAGE and immunoblot with anti P-2 antibody. The studies will also be carried out following P-2-GFP transfection and immunoprecipitation with anti GFP and immunoblotting with anti GFP and anti RASA2 (as in FIG. 35b), respectively. GFP transfection (no P-2 fusion) will serve as control (not shown). The experiments will be carried out with and without IFN pre-induction and also with or without bacterial infection. We will also look for additional protein bands by protein staining of P-2-GFP immunoprecipitates and characterize them by mass spectrometry. GFP immunoprecipitates will serve as controls. We will also undertake mutational analysis of the cytoplasmic domain of P2 and mutation of RASA2.

Direct biochemical interaction between P-2-GFP and RASA2 has now been verified by coimmunoprecipitation in P-2-GFP transfected RAW cells (FIG. 35b) which will allow us to undertake the mutational experiments (GFP control, not shown, did not coimmunoprecipitate RASA2). This will enable us to probe the immunoprecipitates for the presence of Rab5 or vsp34 and autophagy proteins (aim 2) which may be in complex with P-2/RASA2. We will also analyze P-2-GFP immunoprecipitates (using GFP precipitates as control) by SDS PAGE followed by protein staining of the gel to determine the presence of protein bands that do not correspond to the components mentioned so far. If additional bands are present, we will analyze them by mass spectrometry and determine their identity.

Mutational Analysis of RASA2 in Bacterial Killing.

RASA2 has two C2 domains (C2A and C2B) in the N-terminal segment followed by the RasGap domain and the PH/Btk domain towards the C-terminus. C2 domains are protein structural domains involved in targeting proteins to cell membranes, however their function in RASA2 has not been explored. The PH-Btk domains are required for PIP binding. RASA-2 binds to soluble IP(3,4,5)P3, PI(1,3,4,5,6)P5, IP6, but not to IP(1,4,5)P3; RASA2 also binds to PtdIns(3,4,5)P3, indicating that inositol-3 phosphorylation is required for its binding. Whether RASA2 binds PtdInsP(3)P which is constitutively synthesized by vps34 on endogenous membranes is not known.

To examine the role of the functional domains of RASA2 in bacterial killing and in P-2 translocation to the bacterium containing vacuole (FIG. 32), we will delete functional domains: ΔC2A, ΔC2B, ΔC2A,B, ΔRasGAP, ΔPH-Btk. For the analysis endogenous RASA2 will be knocked down with siRNA targeting the 3′UTR and the cells reconstituted with the deletion construct of RASA2 that lacks the 3′UTR (as in FIG. 30 shown for P-2-RFP).

Effect of Bacterial Species on P-2 and RASA2 Translocation.

P-2-translocation in FIG. 35 was done by infection with S. typhimurium and the lab strain E. coli K12. We will also determine translocation of w.t and mutated P-2 and w.t and mutated RASA2, respectively, with Salmonella and Mycobacteria and other bacteria. Mycobacteria interfere with PI3P generation and vacuole maturation. The Salmonella type3 secretion effector SopB has multiple effects on PI3P that interfere with vacuole maturation and enhance intracellular survival in the salmonella containing vacuole, SCV. Changes in PI3P on the salmonella containing vacuole may affect both P-2 and RASA2 translocation in comparison to E. coli.

We expect both, the PH-Btk domain and the RasGap domain of RASA2 to be essential for bacterial killing. Translocation of RASA2 to the bacterium-containing vacuole upon PIP3 generation is expected. RasGap usually acts as switch regulator and may function in translocation of P-2 to the bacterium containing vacuole. RasGap or the C2 domains may be required for P-2/RASA2 interactions which will be assayed by coimmunoprecipitation and by yeast two hybrid analysis.

In subsequent studies we will use RASA2 and its deletion mutants as bait in a yeast two hybrid screens to identify additional candidates assisting RASA2 function, such as Rabs and vacuolar sorting nexins.

Mutational Analysis of the Cytoplasmic Domain of P-2-RFP.

The conserved (highlighted) amino acids in the mouse P-2 cytoplasmic sequence are (RKYKKK- SEQ ID NO:4) followed by a conserved Y, then an acidic EIEEQE (SEQ ID NO:5) followed by a conserved S and an additional S close to the C-terminus (FIG. 28a). Several kinase-families were identified using algorithms for potential phosphorylation sites. We propose that the cytoplasmic domain of P-2 receives signals upon bacterial infection that provide for P-2 translocation to the bacterium containing vacuole and for P-2 polymerization to deliver lethal hits to bacterial cell walls. We will mutate (a) KYKK (SEQ ID NO:7) to QYQQ (SEQ ID NO:8); (b) the conserved Y to F and (c) the two conserved S to A (blocking) or D (constitutively active) (see FIG. 28a). We have already established that the Y to F mutation in P-2-RFP is stably expressed as determined by flow cytometry but abolishes bacterial killing activity when used for complementing siRNA knock down of endogenous P-2.

We will determine the effect of P-2-RFP mutations on expression and stability, on bacterial killing and on translocation of P-2 to bacteria containing vacuoles. In addition we will determine the effect of P-2 mutation on interaction with RASA2 by coimmunoprecipitation (FIG. 35b) and on potential RASA2 translocation to the bacterium containing vacuole.

Mutation of KYKK to QYQQ or of the conserved Y to F (FIG. 28a) in P-2-RFP results in loss of P-2-RFP killing activity but normal levels of expression by flow cytometry (data not shown). In these experiments endogenous P-2 was knocked down by transfection with siRNA specific for the 3′UTR of endogenous P-2 and concurrent transfection with mutated P-2-RFP plasmid-cDNA lacking the native 3′UTR. Interaction of mutated P-2 with RASA2 will be determined by coimmunoprecipitation (FIG. 35b). Mutation of the conserved S to D (imitating phosphorylation) increased P-2 killing activity (data not shown). Other mutations will delete increasing parts of the C-terminal sequence and analyze function in killing and and RASA2 interaction. We anticipate that mutations that result in failure of P-2 interaction with RASA2 will block P-2 mediated killing. However, there may be mutations that do not interfere with RASA2 interaction but still block P-2 function. The latter effect may be due functions of the cytoplasmic domain that trigger P-2 polymerization but do not affecting RASA2 interaction and translocation.

### Example 6

### Analysis of Autophagy as the Link to P-2-Mediated Killing of Intracellular Bacteria

Autophagy (Xenophagy) is activated by infection. The intracellular initiation site of autophagy is defined by phosphorylation of inositol at the 3-position to PI(3,4,5)P3 and PI3P by the vps34 complex which defines the nucleation site of the phagophore that grows into the autophagosome.

After infection RAB5 recruits the PI3-kinase vps 34 to the bacterium containing vacuole that phosphorylates the 3-position of inositol to generate PI(3,4,5)P2 and PI(3)P. The gef for autophagy membrane-vesicle transport is the TRAPPIII complex and we will study interaction of its components withVps34, RASA2, P-2 and Rab1. Vps34-phosphorylation allows binding of RASA2 potentially together with interacting P-2 (FIG. 35b) and also serves as nucleation site for the incipient autophagosome in non-phagocytic cells. In phagocytic cells the same sequence may bring RASA and P-2 to the bacterium containing vacuole which matures into the phagosome. Pathogenic bacteria such as Salmonella enterica serovar typhimurium and Mycobacterium tuberculosis have virulence genes that manipulate these early steps in autophagy or phagosome maturation.

LC3 is an excellent marker for autophagy in mammalian cells associated with early and late autophagosomes. LC3 ligation to phosphatidyl-ethanolamine is catalyzed by the E3 like Atg16L-Atg17-Atg5 complex. The Atg16L-complex is also required for formation of the autophagy double membrane together with Atg9L1 that is required to prevent intracellular replication of S. typhimurium in mEF. The final step is acidification and fusion with lysosomes. We propose that P-2 is recruited to the autophagosome and may be colocalized with LC3 (FIG. 32).

Define the Entry Point of P-2 into the Autophagosome.

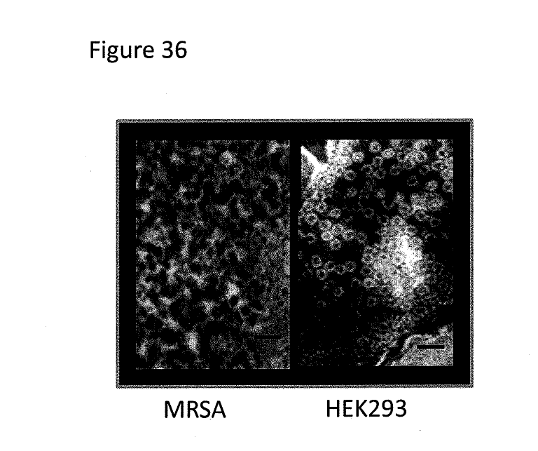

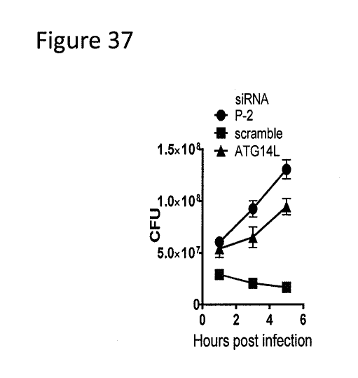

Autophagy is mediated by the stepwise assembly of the autophagy membrane and subsequent fusion with lysosomes. We propose that during infectious autophagy P-2 is recruited to the bacterium containing vacuole and/or incipient autophagosome, where it delivers the lethal hit to bacteria while remaining tethered to the P-2-membrane through the transmembrane domain (see FIG. 28a). P-2 killed bacteria bear cell wall pores strongly suggesting perforation by Perforin-2-polymerization (FIG. 37). MRSA obtained from mEF by detergent lysis 4 h after infection and analyzed by electron microscopy show typical 90 Å cell wall pores (left panel, FIG. 36) similar in size to P-2-pores on eukaryotic membranes (right panel, FIG. 36). Putative P-2 pores are also present on cell walls of mEF-killed mycobacteria (data not shown). P-2 pores may facilitate penetration of ROS, NO and lysozyme across bacterial cell walls to complete killing. According to our proposal of a P-2/autophagy link, blocking of autophagy at steps prior to recruitment of P-2 will block bacterial killing. Blocking autophagy steps after P-2 has been recruited and polymerized will not impair killing. Atg14L is the targeting component of the vps34 PI3-kinase complex in autophagy. Similar to P-2 knock-down, siRNA knock-down of Atg14L blocks killing of Mycobacteria in BV2 despite the presence of fully active P-2 (FIG. 37).

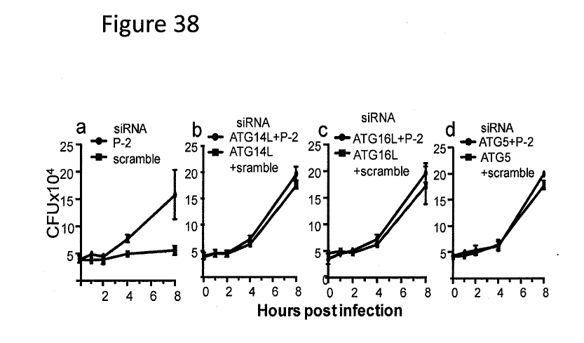

BV2 are phagocytic cells. The data in FIG. 37 suggest that vps34/Atg14L is required for killing of bacteria by phagocytic cells similar to its requirement in mEF (FIG. 38b) that rely on autophagy. The early steps in phagocytosis and autophagy of bacteria may be similar and reflect a common mechanism for P-2 recruitment in non-phagocytic and phagocytic cells. The data are consistent with the hypothesis that P-2 is the effector for killing of bacteria by both, autophagy and phagocytosis. P-2 is recruited, respectively, together with initiation of autophagy or initiation of vacuole maturation into the phagosome.

To further define the point of entry of P-2 into the autophagosome, we will knock down Atg5 and Atg16L1, components of autophagy required for conjugation of LC3 to phosphatidyl-ethanolamine on autophagy-membranes.

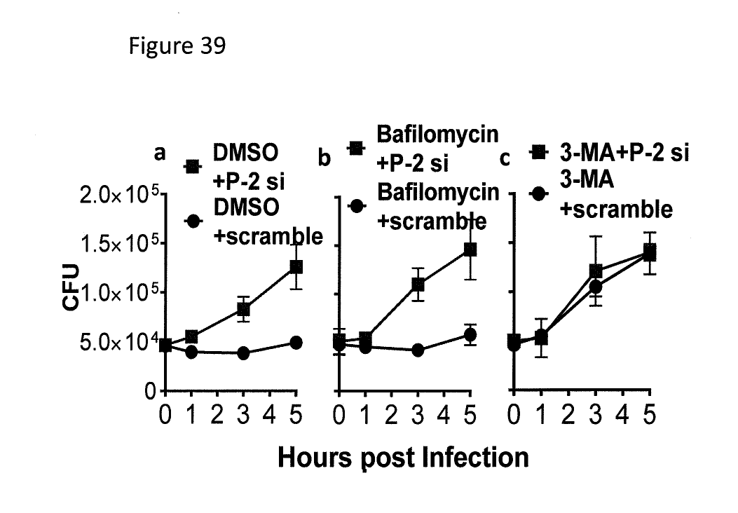

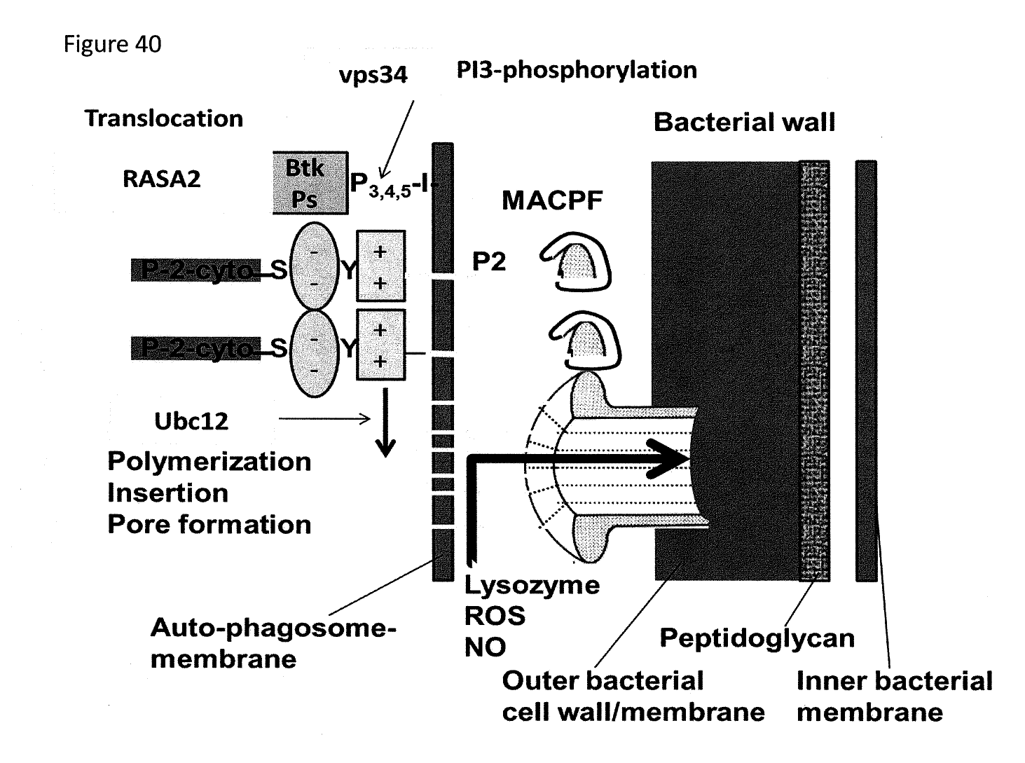

The data in FIG. 38 suggest that Atg14L (FIG. 38b), Atg16L (FIG. 38c) and Atg5 (FIG. 38d) are required for killing and keeping Salmonella bacteriostatic, in agreement with. Their knock-down allows intracellular Salmonella replication in mEF. Atg14L also is required to prevent Salmonella replication in mEF (FIG. 38). Atg14L is a component of the PI3-kinase-vps34 complex which initiates autophagy. To confirm the involvement of vps34-PI3-kinase enzymatic activity, we will use PI3-kinase blockers, Wortmannin or the more selective vps34 inhibitor 3-methyl-adenine (3-MA), both of which are known to block autophagy. 3-MA similar to P-2 knock down permits Salmonella replication suggesting that the enzymatic activity of vps34 is required for intracellular killing of bacteria. In contrast, Bafilomycin does not interfere with bactericidal/bacteriostatic activity. Bafilomycin prevents acidification and lysosome fusion, which is not required for killing of Salmonella by mEF (FIG. 39).

Both, P-2 knock down and autophagy blockade prevent intracellular killing of bacteria allowing their replication in mEF. The simplest explanation for this finding is that that both processes are required for bacterial control and may be linked. P-2 may deliver the lethal hit to bacteria in concert with autophagy. The data in FIG. 37-39 place P-2 action after Atg14L-vps34 but before phagosome-lysosome fusion. Knock down of Atg5 or Atg16L allows replication of Salmonella in mEF in agreement with published reports. Atg5 is required as part of the Atg16L complex ligating LC3 to phosphatidyl-ethanolamine. Therefore, LC3 lipidation may be required for P-2 mediated killing of Salmonella. The Atg16L complex together with Atg9L1 is also required for formation of the double membrane. The absence of Atg9L1 in knockout mEF promotes intracellular Salmonella replication. Atg9L1 may be one of the main lipid-vesicle donors required together with the Atg16L complex for formation of the autophagy double membrane which is decorated with LC3. Atg9 is a multi-spanning membrane protein residing in 300-600 Å diameter membrane vesicles present in the cytoplasm that, in yeast, are derived from the Golgi complex with the help of Atg23 and Atg27. Homologues of yeast Atg23 and 27 have not been described in mammals. Atg9L1 is the mammalian homologue of yeast Atg9. It is possible that the transmembrane protein P-2 (FIG. 28a) is recruited by RASA-2 together with Atg9L1-vesicles to the bacterium containing vacuole in a process that requires Atg16 and LC3 and initiates formation of the double membrane. P-2-vesicles could fuse with Atg9L1-vesicles or be recruited separately by the same transport pathway that may include RASA2, Rab5 or Rab7 and/or other components.

Define P-2 Translocation and LC3 in Autophagy by Imaging.

FIG. 32 shows examples of P-2 translocation to the bacterium containing vacuole within 5 min of infection. In that experiment endogenous P-2 was knocked down with siRNA and at the same time the cells transfected with P-2-RFP. 16 h later the cells were infected with GFP-expressing E. coli K12 and fixed with paraformaldehyde 5 min post infection and imaged by confocal microscopy. FIG. 32a shows that P-2 and LC3 are colocalized on the bacterium containing vacuole within 5 min of infection.

To measure LC3 ligation to phagosomes or autophagosomes, we will per-manently transfect mEF, BV2 and RAW with LC3-GFP. Our studies will focus primarily on naïve and on IFN induced mEF; BV2 and Raw cells will be used as controls. Atg 5, 7, 9L1, 14L or 16L will be knocked down and the cells infected with Salmonella, MRSA or Mycobacteria and the reaction stopped by fixation with.

Study in IFN Induced mEF the Relationship Between P-2 and ATG9L1 in Bacterial Killing by Knock Down and Imaging.

ATG9L1 is required for slowing intracellular replication of Salmonella in mEF not induced to express P-2 by IFN. We have shown that bacteria will replicate in mEF when of P-2 is not present and in FIG. 34b that Salmonella suppress P-2 induction. We will therefore study the role of ATG9L1 by imaging in w.t. and ATGL1 knock down cells, induced or not induced for P-2 expression. We will also look for coimmunoprecipitation with P-2-GFP or/and RASA2. Since ATG9L1 is a 6-membrane spanning protein embedded in ˜60-90 nm lipid vesicles it is a potential candidate for interacting with P-2 and using similar translocation pathways.

1. A method of modulating function, activity or expression of Perforin-2 (P2) comprising: administering to a patient, an effective amount of at least one agent which modulates the function, activity or expression of one or more molecules associated with P2 expression, function or activity; and, modulating the function or expression of P2.

2. The method of claim 1, wherein the one or more molecules associated with P2 function, activity or expression comprise: src, ubiquitin conjugating enzyme E2M (Ubc12), GAPDH, P21RAS/gap1m (RASA2), Galectin 3, ubiquitin C (UCHL1), proteasomes, vps34, ATG5, ATG7, ATG9L1, ATG14L, ATG16L, LC3, Rab5, or fragments thereof.

3. The method of claim 1, wherein the molecule inhibits transcription or translation of P2.

4. The method of claim 1, wherein an agent comprises: a small molecule, protein, peptide, polypeptide, modified peptides, modified oligonucleotides, oligonucleotide, polynucleotide, synthetic molecule, natural molecule, organic or inorganic molecule, or combinations thereof.

5. A method of identifying a candidate therapeutic agent comprising: contacting a cell expressing one or more target molecules comprising: src, ubiquitin conjugating enzyme E2M (Ubc12), GAPDH, P21 RAS/gap1m (RASA2), Galectin 3, ubiquitin C (UCHL1), proteasomes, vps34, ATG5, ATG7, ATG9L1, ATG14L, ATG16L, LC3, Rab5, or fragments thereof; measuring the expression, function or activity of the molecules; comparing the expression, function or activity of the molecules with a control; and, identifying a candidate therapeutic agent which modulates expression, function or activity of Perforin-2.

6. The method of claim 5, wherein the modulation of the expression, function or activity of one or more target molecules modulates the expression, function or activity of Perforin-2 (P2) molecules.

7. The method of claim 5, wherein the target molecules are polynucleotides or expressed products thereof.

8. A method of identifying a candidate therapeutic agent comprising: contacting an assay surface with one or more target molecules comprising src, ubiquitin conjugating enzyme E2M (Ubc12), GAPDH, P21RAS/gap1m (RASA2), Galectin 3, ubiquitin C (UCHL1), proteasomes, vps34, ATG5, ATG7, ATG9L1, ATG14L, ATG16L, LC3, Rab5, fragments or associated molecules thereof; contacting the target molecules with one or more candidate therapeutic agents and identifying the agents which bind or hybridize to one or more target molecules or associated molecules thereof.

9. The method of claim 8, wherein the identified candidate therapeutic agents are assayed for modulation of expression, function or activity of Perforin-2 molecules.

10. The method of claim 9, wherein the identified candidate agents are assayed for inhibition of replication, inhibition of growth, or death of an infectious organism.

11. The method of claim 10, wherein the infectious organism is an intracellular or extracellular bacterium.

12. A method of treating a patient suffering from an infectious disease organism comprising, administering to the patient a therapeutically effective amount of an agent identified by the method of claim 8.

13. A transgenic mouse which comprises a disruption of a gene encoding a Perforin-2 protein.

14. The transgenic mouse of claim 13, wherein said disruption comprises a heterozygous or homozygous disruption of said gene encoding a Perforin-2 protein.

15. The transgenic mouse of claim 14, wherein said disruption comprises a homozygous disruption, wherein said homozygous disruption inactivates said gene and inhibits the expression of a functional Perforin-2 protein in said transgenic mouse.

16. The transgenic mouse of claim 13, wherein said transgenic mouse exhibits an increased susceptibility to infection by intracellular pathogens as compared to a wild-type mouse.

17. An organ, a tissue, a cell, or a cell-line derived from the transgenic mouse of claim 13.


SEQUENCE LISTING (19 sequences)

SEQ ID NO: 1 - RNA, 19 nt, Artificial Sequence
  Synthesized siRNA
ccaccucacuuucuaucaa

SEQ ID NO: 2 - RNA, 19 nt, Artificial Sequence
  Synthesized siRNA
gaguauucuaggaaacuuu

SEQ ID NO: 3 - RNA, 19 nt, Artificial Sequence
  Synthesized siRNA
caaucaagcucuugugcac

SEQ ID NO: 4 - protein, 6 aa, Mus musculus
RKYKKK

SEQ ID NO: 5 - protein, 6 aa, Mus musculus
EIEEQE

SEQ ID NO: 6 - protein, 15 aa, Mus musculus
GTRKYKKKEYQEIEE

SEQ ID NO: 7 - protein, 4 aa, Mus musculus
KYKK

SEQ ID NO: 8 - protein, 4 aa, Artificial Sequence
  Synthesized
QYQQ

SEQ ID NO: 9 - protein, 716 aa, Homo sapiens
MNNFRATILFWAAAAWAKSGKPSGEMDEVGVQKCKNALKLPVLEVLPGGGWDNLRNVDMGRVMELTYSNCRTTEDGQYIIPDEIFTIPQKQSNLEMNSEILESWANYQSSTSYSINTELSLFSKVNGKFSTEFQRMKTLQVKDQAITTRVQVRNLVYTVKINPTLELSSGFRKELLDISDRLENNQTRMATYLAELLVLNYGTHVTTSVDAGAALIQEDHLRASFLQDSQSSRSAVTASAGLAFQNTVNFKFEENYTSQNVLTKSYLSNRTNSRVQSIGGVPFYPGITLQAWQQGITNHLVAIDRSGLPLHFFINPNMLPDLPGPLVKKVSKTVETAVKRYYTFNTYPGCTDLNSPNFNFQANTDDGSCEGKMTNFSFGGVYQECTQLSGNRDVLLCQKLEQKNPLTGDFSCPSGYSPVHLLSQIHEEGYNHLECHRKCTLLVFCKTVCEDVFQVAKAEFRAFWCVASSQVPENSGLLFGGLFSSKSINPMTNAQSCPAGYFPLRLFENLKVCVSQDYELGSRFAVPFGGFFSCTVGNPLVDPAISRDLGAPSLKKCPGGFSQHPALISDGCQVSYCVKSGLFTGGSLPPARLPPFTRPPLMSQAATNTVIVTNSENARSWIKDSQTHQWRLGEPIELRRAMNVIHGDGGGLSGGAAAGVTVGVTTILAVVITLAIYGTRKFKKKAYQAIEERQSLVPGTAATGDTTYQEQGQSPA

SEQ ID NO: 10 - protein, 717 aa, Bos taurus
MNSFRGAFLIWAVATWAETDTSWGATDEPGFQNCKNALKLPVLPVLPGGGWDNLRNVDTGRVMELAYSHCRTTEDGQYIVPDEIFTIPQKQSNLEMNSKILESWVNYQSSTSNSINMELSLFSKVNGKFSLEFQRIKTLQVKDQAVTTQVQVRNLVYTVKINPDAELSLGFKKALMDISEQLENNQTRMATYLAELLVLNYGTHVITSVDAGAALIQEDHIRSSFLQDSQSSRSAVTASAGITFLNIVNFKFEENYTSQNTFTKSYLSNRTNSRVQSFGGLPFYPGITLQAWQQGVSNHLVAMDRAGLPLYFFINPERLPDLPGPLVRKLSKTVEAAVRRYYAVNTYPGCTDLSSPNFNFQANTDDGSCEGKMTNFSFGGVYQECTQFSGNEVVQLCQNLEQKNPLTGSVSCPSGYSPVQLLTQTHEEGYNHLECSRKCTLYIFCKTVCEDVFRVARAEFRAFWCAASGQVSENSGLLFGGLFSGKSINPLTNAQSCPAGYFQLKLFENLKVCASLDYELGYRFSIPFGGFFSCAAGNPLVDSATSKDLGAPSLRKCPGGFSQHLALISDGCQVSYCVKAGLFTGGSLPPVRLPPYTRPPLMSQVATNTVLVTNHETASSWIKDPQTHQWRLGEPLELRRAMRVVHGDGEGLSGGAAAGLTLGVTIALAGVVALAIYGARKSRKKGYQALQDEKQSLAAGAAVNGDALDQEQAQNPA

SEQ ID NO: 11 - protein, 715 aa, Canis familiaris
MSSVRGAILFWVVVAWAKTDKPLEQTNETGFQKCKNALKLPVLEVLPGGGWDNLRNVDMGRVMDLTYRSCRTTEDGQYIIPDEITSIAQKQSNLEMNSEILESWVNYQSSTSSSINLELSLYSKVNGKFSSDFQQMKTLQVKDQAITTRVQIRNLIYTVKINSASKLSWGFKKDLMDISDRLENNQTRMATYLAELLVLNYGTHVVTSVDAGAALLQEDHIRASFLQDSQSSHTAVTASAGVAFMNVVNYKFEENYTSQNALTKSYLANRTHSRVRSIGGVPFYPGITLQAWQQSIANHLVAIDRAGLPLPFFISPDTLPELPGPLVKKLSKTVEAAVRHYYAFNTYPGCTDANSPNFNFQANTDDGSCEGKMTNFSFGGVFQECTQLSGKEAAQLCQTLEQRNPLTGAFSCPSGYSPIHLLSQVHEEGYNHLECRRKCTLLVFCKTVCEDVFRVAKAEFRAFWCVASGQIPENSGLLFGGLFSGKTINPLTNAQSCPAGYFPLRLFENLKVCASLDYELGFRFSVPFGGFFSCAVGNPLVNSAFTEGAPSLKKCPGGFSQHLALISDGCQVSYCVKSGLFTGGSLPPARLPPYTRPPLMSQAATNTVIVTNSETASSWIKDSQTRQWRLGEPLELRRAMKVIRGDGGGLSGGAAAGVTMGVTTVLAAVIALAIYGTRKYKKRGYQALEDERQSLAAGTAESGDAPGQEQEQSPA

SEQ ID NO: 12 - protein, 720 aa, Mus musculus
MAKTICAMNSFMALVLIWMIIACAEADKPLGETGTTGFQICKNALKLPVLEVLPGGGWDNLRNVDMGRVMDLTYTNCKTTEDGQYIIPDEVYTIPQKESNLEMNSEVLESWMNYQSTTSLSINTELALFSRVNGKFSTEFQRMKTLQVKDQAVTTRVQVRNRIYTVKTTPTSELSLGFTKALMDICDQLEKNQTKMATYLAELLILNYGTHVITSVDAGAALVQEDHVRSSFLLDNQNSQNTVTASAGIAFLNIVNFKVETDYISQTSLTKDYLSNRTNSRVQSFGGVPFYPGITLETWQKGITNHLVAIDRAGLPLHFFIKPDKLPGLPGPLVKKLSKTVETAVRHYYTFNTHPGCTNVDSPNFNFQANMDDDSCDAKVTNFTFGGVYQECTELSGDVLCQNLEQKNLLTGDFSCPPGYTPVHLLSQTHEEGYSRLECKKKCTLKIFCKTVCEDVFRVAKAEFRAYWCVAAGQVPDNSGLLFGGVFTDKTINPMTNAQSCPAGYIPLNLFESLKVCVSLDYELGFKFSVPFGGFFSCIMGNPLVNSDTAKDVRAPSLKKCPGGFSQHLAVISDGCQVSYCVKAGIFTGGSLLPVRLPPYTKPPLMSQVATNTVIVTNSETARSWIKDPQTNQWKLGEPLELRRAMTVIHGDSNGMSGGEAAGITLGVTIALGVVITLAIYGTRKYKKKEYQEIEEQESLVGSLATDATVLNGEEDPSPA

SEQ ID NO: 13 - protein, 718 aa, Danio rerio
MKSRAFHLLMLCCFISVCNLHPLIRPNNGLRLCRKNSSLTALEVLPGGGWDNLRNIDMGRVMNLSYSQCQTTEDGVYLIPDEVFVIPQKVSGVETNSEIIMSWLEQKSSTSSSVNADVSFFSVLNAKFSTENQRMKTHQVKEGSVTARVQVRNHLYTVKAYPDFTLDSRFAKQAEEIADAIENNQTRHANYLSEKLVLDYGTHVITSVDAGATLVQEDYLKMSYISNSQSDKSSVSASAGANFFDKVKFDIGGNTSQGSSQSSSYQGNITYSLIQSHGGALFYPGITLQKWQQSTLNNLAAIDRSGLPLHYFLNPSTFPDLPTPTVNKLASTVRKAAERYYKVNTIPGCVNVDSPNFNFQANVDDASCEGPITNLSFGGIYQKCTPLTPDGNIICDETAQKNPATGGYSCPQHYNTTLLHSEVVEKGFNHYECHTHCHSCGFLGLSTCCDKTCGDSYHVRRAKLETLWCSSTHKTPENSGYLFGGLFGPGIQNPLTKSSSCPPSYFTQRFLSNGMMICMSNDYEIGTRFSVPFAGFFSCQSGNPLSNGQSRCPPQFSQHLAAISDGCQVLYCVQSGVFSGGHLKPVRLPPFTRPPVVGMIATNTVAVMTEGERSWVRVGETKMWRLAKPGDIKQMQSILDASEMSGGKKAGVAIGIIVLVALVVAGTVVIMKRRNRFSSLKLNRGYEEISEERNESSVEIEQEQNEAANENPNQQLLS

SEQ ID NO: 14 - protein, 730 aa, Haliotis rufescens
MLCFVFGVSIVAGVIGGELLNTVQKPEFPKGDVRACYGDNKKLERFEVLPGQGWDNLRNVDAGLVVVYNYSRCRTTEDGRFLIPDTVNTIPLKASKLNVYAELISHWSNYTSTTAHGVNIDAGLKFGSVKVSGTFSSGYESVKSKQIGDKSYTTRVQLRYVRYSAKLQPDAALHPTFKSRLLSIAGSLQLNKTDQARYDSELLVRDFGTHVVTSVDAGAALVQEDQVSSEFVNSRKFTKNQITAGASASFLGIFSIDVSYHSSTSNEVKTAYEKSRSSSQIDTLGGPMFKASNFTANDWTNEVDHELVAVDRSGDPLFFLINSASLPELPNSVLYQLQNLVEETILHYYEFNTYRGCTELDSPNFSPAANLDDGTCKSPYTNLTFGGVYQTCSMSSGSNNGDLCSGLDQVNPKTGGHTCPDGYESVELHTGRLSDSKSVHSCHSCWLFFKCCHDNYYHSEATYIMHWCAATGPVSQDSGYLFGGLYTSQLNNPLTQGKTCPVNFYTRTLGKDLHICISDDYELGMKYSMPFGGFISCTTGNPLAMNPKPKSKGDMNSALPSLHSFFQGSKTWPKHCPKGYSQHLAYVDQGCEINYCLLAGSLSEVGLPKIRRPPFQTAPLLLPSTENHVVFDPVTLTWRKNQEAMQFISARGGDATSSSTGSGMSAGAAAGIAVVATLGCVVISTIIIVLIKRRRKSSAGYRHLAIDDPLLSSQSNYGATGSDAVNVNVE

SEQ ID NO: 15 - protein, 676 aa, Suberites domuncula
MVCLTRQIGSLTDTHPTNKLVMVGGGGGAGGLLSLVSGALSGGGGNIRAGAPRNMYPRGDPRNCLSGNPKLNILQVVPGIGWDNLRNSETGILTSFSYSQCKVTYDRRYLIPDETFAIPIKTSTIDYQAELFDHWDAYKSVTSRSINAGFDFFGKIGGEILSLLKSNDTESADYAAQILIRDYGTHCITSIDAGAVLIKEDNLKSTIMSNYKGRADSLSTAAGVEFYDMLKLRASAGFSSYSGDSDLKAYRQNRTSSRLYTYGGPPYKLGMNLSRWENDLMNNLVATDRSGKPSHSLSTTQSLKPEVTTSQEVFLLRRLVKSAVSQYYHYNTHTGCKNPKAPNLDHQTNNGAPGVCKEPSANYTFGGVFQSCRSNGNDICGKLLQKNPLTGGYSCPKNFKALLLQLGTERSHKMRRVCVWKRKCTFFVFNCHDVDDCTFVPSVEIASYQTYWCAPNKKNPPKFGYMFGGIYSNDIQNPITRSCSCPTHFLPLRMGERATVCVSEDYELGHQFSLPFGGFFSCVSGNVLAGNGSSEFLNNPKDWPMRCPGGFTQHLALTEKGCRVNFCVKAGSLLRASDLELVLPPFDPKPTLRKNSTSDLFSKPAVAPSGSLPIKGVPPPLDNNRVIMYYPVLSSNTGQRDNGVGRITEPFFCHSWDWSVDLSVAHQAIDKFAFAK

SEQ ID NO: 16 - protein, 40 aa, Mus musculus
YGTRKYKKKEYQEIEEQESLVGSLATDATVLNGEEDPSPA

SEQ ID NO: 17 - protein, 715 aa, Canis familiaris; identical to SEQ ID NO: 11

SEQ ID NO: 18 - protein, 41 aa, Equus caballus
YGTRKYKKREYQALEEERQSIAPGTAETRDMPDQEQEQSPA

SEQ ID NO: 19 - protein, 40 aa, Homo sapiens
YGTRKFKKKAYQAIEERQSLVPGTAATGDTTYQEQGQSPA